SEQUENCE LISTING (419 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 69 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 70 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 71 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 72 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 73 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 74-100 - skipped (27 entries)

SEQ ID NO: 101 - protein, 323 aa, synthetic construct
GPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 323 aa, synthetic construct
GPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 104 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 107 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 108 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNRFTQKSLSLSLGK

SEQ ID NO: 110 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 107 aa, synthetic construct [sequence omitted]

SEQ ID NO: 112 - protein, 12 aa, synthetic construct
ESKYGPPCPSCP

SEQ ID NO: 113 - protein, 12 aa, synthetic construct
ESKYGPPCPPCP

SEQ ID NO: 114 - protein, 94 aa, synthetic construct
GPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 115 - protein, 447 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116-223 - skipped (108 entries)

SEQ ID NO: 224 - protein, 20 aa, synthetic construct
  VARIANT 1..20: note = This sequence may encompass 1-10 GS repeating units
GSGSGSGSGSGSGSGSGSGS

SEQ ID NO: 225 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 226 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 227 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 228 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 229 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 230 - protein, 15 aa, synthetic construct
GGGGSGGGGSGGGGS

SEQ ID NO: 231 - protein, 20 aa, synthetic construct
GGGGSGGGGSGGGGSGGGGS

SEQ ID NO: 232 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 234 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 448 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 240-241 - skipped (2 entries)

SEQ ID NO: 242 - protein, 118 aa, synthetic construct
QGQLQESGPSLVKPSQTLSLTCTVSGDSITRGYWNWIRKHPGKGLEYIGYISYTGSTYSNLSLKSRVTISRDTSKNQYYLKLSSVTAADTAVYYCATSTGWLDPVDYWGQGTLVTVSS

SEQ ID NO: 243 - protein, 107 aa, synthetic construct
DIVMTQSPDSLAVSLGERATINCKASQNVDTSVAWFQQKPGQPPKALIYSASFRYSGVPDRFSGSGSGTDFTLTISSLQAEDVAVYFCQQYYGYPFTFGQGTKLEIK

SEQ ID NO: 244 - protein, 123 aa, synthetic construct
EVQLVQSGGGLVKPGGSLRLSCAASGFTFSSYTMNWVRQAPGKGLEWVSSISSGSDYLYYADSVKGRFTISRDNAKNSLYLQMNSLRAEDTAVYYCARNELRWYPQAGAFDRWGQGTMVTVSS

SEQ ID NO: 245 - protein, 109 aa, synthetic construct
QSVVTQPPSMSAAPGQRVTISCSGSSSYIESSYVGWYQQLPGTAPRLLIYDDDMRPSGIPDRFSGSKSGTSATLAITGLQTGDEADYYCEIWRSGLGGVFGGGTKLTVL

SEQ ID NO: 246-247 - skipped (2 entries)

SEQ ID NO: 248 - protein, 453 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 216 aa, synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 250 - protein, 5 aa, synthetic construct
SYTMN

SEQ ID NO: 251 - protein, 17 aa, synthetic construct
SISSGSDYLYYADSVKG

SEQ ID NO: 252 - protein, 14 aa, synthetic construct
NELRWYPQAGAFDR

SEQ ID NO: 253 - protein, 13 aa, synthetic construct
SGSSSYIESSYVG

SEQ ID NO: 254 - protein, 7 aa, synthetic construct
DDDMRPS

SEQ ID NO: 255 - protein, 10 aa, synthetic construct
EIWRSGLGGV

SEQ ID NO: 256 - protein, 360 aa, synthetic construct
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSEPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 759 aa, synthetic construct
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 258 - protein, 942 aa, synthetic construct
QMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSEVQLQQSGPELVKPGPSMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSQMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRQLVLTQPPSVSGAPGQRVTISCTGSSSNIGAGYDVHWYQQLPGAAPKLLIYGDINRPSGVPDRFSGSKSGISASLAITGLQAEDEADYYCQSYDSSLSGGVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259-329 - skipped (71 entries)

SEQ ID NO: 330 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 331 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 332 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 333 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 335 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 339 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 341 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342-345 - skipped (4 entries)

SEQ ID NO: 346 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 349 - protein, 111 aa, synthetic construct [sequence omitted]

SEQ ID NO: 350 - protein, 113 aa, synthetic construct [sequence omitted]

SEQ ID NO: 351 - protein, 107 aa, synthetic construct [sequence omitted]

SEQ ID NO: 352 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 354 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 355 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 357 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 443 aa, synthetic construct
QVQLVESGGGLVKPGGSLRLSCAASGFTFSNYGMSWIRQAPGKGLEWSTISGGGSNIYYADSVKGRFTISRDNAKNSLYLQMNSLRAEDTAVYYCVSYYYGIDFWGQGTSVTVSSASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 361 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 365 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 369 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 370 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 373 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 374 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 375 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 376 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 377 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 378 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380 - protein, 5 aa, synthetic construct
NYYMY

SEQ ID NO: 381 - protein, 17 aa, synthetic construct
GINPSNGGTNFNEKFKN

SEQ ID NO: 382 - protein, 11 aa, synthetic construct
RDYRFDMGFDY

SEQ ID NO: 383 - protein, 15 aa, synthetic construct
RASKGVSTSGYSYLH

SEQ ID NO: 384 - protein, 7 aa, synthetic construct
LASYLES

SEQ ID NO: 385 - protein, 9 aa, synthetic construct
QHSRDLPLT

SEQ ID NO: 386 - protein, 5 aa, synthetic construct
NSGMH

SEQ ID NO: 387 - protein, 17 aa, synthetic construct
VIWYDGSKRYYADSVKG

SEQ ID NO: 388 - protein, 4 aa, synthetic construct
NDDY

SEQ ID NO: 389 - protein, 11 aa, synthetic construct
RASQSVSSYLA

SEQ ID NO: 390 - protein, 7 aa, synthetic construct
DASNRAT

SEQ ID NO: 391 - protein, 9 aa, synthetic construct
QQSSNWPRT

SEQ ID NO: 392 - protein, 9 aa, synthetic construct
FTFSNYGMS

SEQ ID NO: 393 - protein, 10 aa, synthetic construct
TISGGGSNIY

SEQ ID NO: 394 - protein, 9 aa, synthetic construct
VSYYYGIDF

SEQ ID NO: 395 - protein, 11 aa, synthetic construct
KASQDVTTAVA

SEQ ID NO: 396 - protein, 7 aa, synthetic construct
WASTRHT

SEQ ID NO: 397 - protein, 9 aa, synthetic construct
QQHYTIPWT

SEQ ID NO: 398 - protein, 8 aa, synthetic construct
GLTFSSSG

SEQ ID NO: 399 - protein, 8 aa, synthetic construct
IWYDGSKR

SEQ ID NO: 400 - protein, 6 aa, synthetic construct
ATNNDY

SEQ ID NO: 401 - protein, 11 aa, synthetic construct
RASQSVSSYLA

SEQ ID NO: 402 - protein, 7 aa, synthetic construct
TASNRAT

SEQ ID NO: 403 - protein, 9 aa, synthetic construct
QQYSNWPRT

SEQ ID NO: 404 - protein, 10 aa, synthetic construct
GYTFTSYYMY

SEQ ID NO: 405 - protein, 17 aa, synthetic construct
GVNPSNGGTNFNEKFKS

SEQ ID NO: 406 - protein, 11 aa, synthetic construct
RDYRYDMGFDY

SEQ ID NO: 407 - protein, 15 aa, synthetic construct
RASKGVSTSGYSYLH

SEQ ID NO: 408 - protein, 6 aa, synthetic construct
LASYLE

SEQ ID NO: 409 - protein, 9 aa, synthetic construct
QHSRELPLT

SEQ ID NO: 410 - protein, 5 aa, synthetic construct
GGGGS

SEQ ID NO: 411 - protein, 218 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 447 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNRFTQKSLSLSLGK

SEQ ID NO: 413 - protein, 619 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSGGGGSYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 414 - protein, 390 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEDGGGGSGGGGSGGGGSEIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 415 - protein, 619 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 416 - protein, 390 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 417 - protein, 619 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEDGGGGSGGGGSGGGGSQVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 418 - protein, 619 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEDGGGGSGGGGSGGGGSQVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 419 - protein, 401 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEDGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

(19) United States
(12) Patent Application Publication
(10) Pub. No.: US 2025/0188166 A1
(43) Pub. Date: Jun. 12, 2025

(54) IL-18 POLYPEPTIDES FUSED TO IMMUNE CELL ANTIGEN SPECIFIC BINDING POLYPEPTIDES AND USES THEREOF

(71) Applicant: BRIGHT PEAK THERAPEUTICS AG, Allschwil (CH)

(72) Inventors: Vijaya Raghavan PATTABIRAMAN, Volketswil (CH); Bertolt KREFT, Kleinmachnow (DE); Arnaud GOEPFERT, Sierentz (FR); Olivier BORNERT, Baldenheim (FR); Jean-Philippe CARRALOT, Saint-Louis (FR); Kea MARTIN, Istein (DE)

(21) Appl. No.: 18/813,286

(22) Filed: Aug. 23, 2024

Related U.S. Application Data
(60) Provisional application No. 63/534,311, filed on Aug. 23, 2023.

(51) Int. Cl. C07K 16/28; C07K 14/54

(52) U.S. Cl. CPC C07K 16/2827; C07K 14/5434; C07K 2317/76; C07K 2317/92; C07K 2319/33; C07K 2319/75

(57) ABSTRACT

The present disclosure relates to fusion immunocytokines comprising antibodies or antigen binding fragments specific for immune cell associated antigens, such as PD-1, and IL-18 polypeptides. Also provided herein are methods of treatment with and methods of manufacture of such fusion immunocytokines.

### CROSS REFERENCE

This application claims the benefit of U.S. Provisional Application No. 63/534,311 filed Aug. 23, 2023, which applications is incorporated herein by reference in their entirety.

### SEQUENCE LISTING

The instant application contains a Sequence Listing which has been submitted electronically in XML format and is hereby incorporated by reference in its entirety. Said XML copy, created on Aug. 13, 2024, is named 94917-0150_736201US_SL.xml and is 260,072 bytes in size.

### BACKGROUND

Cancer and other proliferative disorders effect many people in the United States and globally. Immunotherapies utilize the immune system of a subject to aid in the treatment of ailments. Immunotherapies can be designed to either activate or suppress the immune system depending on the nature of the disease being treated. A goal of various immunotherapies for the treatment of cancer is to stimulate the immune system so that it recognizes and destroys tumors or other cancerous tissue.

Immune cells implicated in response to various cancers express certain proteins that are implicated in the regulation of the immune response. These proteins, such as programmed cell death protein 1 (PD-1), programmed death-ligand 1 (PD-L1), and others, can downregulate the immune system and promote self-tolerance by suppressing T cell inflammatory activity. In light of these mechanisms, antibodies or antigen binding fragments which target these proteins have been identified as potential therapeutics. However, in some cases single mechanism therapies targeting these proteins alone are insufficient for treating cancer. Thus, there is a need for improved tools for cancer therapy.

### BRIEF SUMMARY

Described herein are fusion immunocytokines which contain an immune cell associated antigen specific antibody or antigen binding fragment thereof fused to an interleukin 18 (IL-18) polypeptide.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A illustrates a non-limiting mechanism of action of an anti-PD-1 antibody/IL-18 immunocytokine provided herein, wherein an activated T cell shows enhanced activation through concurrent blockade of PD-1 and stimulation by IL-18.

FIG. 1B illustrates a non-limiting mechanism of action of an anti-PD-L1 antibody/IL-18 immunocytokine provided herein, wherein the immunocytokine disrupts PD-L1/PD-1 interaction and effectuates IL-18R signaling.

FIG. 1C illustrates a non-limiting mechanism of action of an anti-PD-L1 antibody/IL-18 immunocytokine acting on an immune cell expressing both IL-18R and PD-L1.

FIGS. 2A-2F show depictions of exemplary formats of fusion immunocytokines described herein. In each figure, the IL-18 polypeptide is denoted by the hexagon shape.

FIG. 2A shows a fusion immunocytokine with a single IL-18 polypeptide fused via its N-terminus to the C-terminal end of one of the heavy chains of the antibody.

FIG. 2B shows a fusion immunocytokine with two IL-18 polypeptides fused to the antibody, both IL-18 polypeptides fused via their respective C-terminuses to the N-terminal ends of the heavy chains of the antibody (i.e., the N-terminal end of the heavy chain variable region).

FIG. 2C shows a fusion immunocytokine with two IL-18 polypeptides fused to the antibody, both IL-18 polypeptides fused via their respective N-terminuses to the C-terminal ends of the heavy chains of the antibody (i.e., the C-terminal end of the CH3 domain).

FIG. 2D shows a fusion immunocytokine with a single IL-18 polypeptide fused via its C-terminus to the N-terminal end of one of the heavy chains of the antibody (i.e., the—terminal end of the heavy chain variable region).

FIG. 2E shows a fusion immunocytokine with two IL-18 polypeptides fused to the antibody, both IL-18 polypeptides fused via their respective N-terminuses to the C-terminal ends of the light chains of the antibody (i.e., the C-terminal end of the light chain constant region).

FIG. 2F shows a fusion immunocytokine with a single IL-18 polypeptide fused via its C-terminus to a fragment crystallizable (Fc) domain (i.e., fused to the N-terminus of a CH2 domain) which is paired with a monovalent antibody domain.

FIG. 3 illustrates the mechanism of action of IL-18 on IFNγ and IL-18BP production, and IL-18 inhibitory activity by IL-18BP.

FIG. 4A and FIG. 4B show plots measuring ability of the unmodified and of conjugated anti-PD1 antibodies to bind with human PD1/CD279 ligand, with the figure showing ELISA signal on the y-axis and dosage of the biotinylated PD-1 protein on the x-axis.

FIG. 4A shows the unconjugated reference antibodies. Tested in this figure are Pembrolizumab, LZM-009, Nivolumab, Atezolizumab, Durvalumab, and Avelumab.

FIG. 4B shows the conjugated antibodies. Tested in this figure are compositions A and composition B.

FIG. 5A and FIG. 5B show plots measuring ability of the unmodified and of conjugated antibodies to bind with human PD-L1/B7-H1 ligand, with the figure showing ELISA signal on the y-axis and dosage of the biotinylated PD-L1 protein on the x-axis.

FIG. 5A shows the unconjugated reference antibodies. Tested in this figure are Pembrolizumab, LZM-009, Nivolumab, Atezolizumab, Durvalumab, and Avelumab.

FIG. 5B shows the conjugated antibodies. Tested in this figure are compositions A and composition B.

FIG. 6 shows plots measuring ability of the unmodified and of conjugated antibodies to bind to human PD-L1/B7-H1 ligand, with the figure showing net BioLayer interferometry shift in nanometer on the y-axis and time of incubation dosage of the biotinylated PD-L1 protein on the x-axis. The figure shows mean ELISA signal on the y-axis and dosage of the human Fc gamma receptors on the x-axis. The unconjugated reference antibodies are Pembrolizumab and LZM-009. The conjugated antibodies tested is Compositions A.

FIG. 7A and FIG. 7B show plots measuring ability of the unmodified and of conjugated anti-PD1 antibodies to interfere with PD1/PDL1 pathway, with the figure showing normalized luminescence intensity of effector cells NFAT-Lucia reporter on the y-axis and dosage of the unmodified and of conjugated anti-PD1 antibodies on the x-axis.

FIG. 7A shows the unconjugated reference antibodies are Pembrolizumab, LZM-009, Nivolumab, Atezolizumab, Durvalumab, and Avelumab.

FIG. 7B shows the conjugated antibodies tested. Tested in this figure are compositions A and composition B.

FIG. 8A shows plots measuring ability of the unmodified and of conjugated antibodies to bind to human Fc gamma receptor I (CD64). The figure shows mean ELISA signal on the y-axis and dosage of the human Fc gamma receptors on the x-axis. The unconjugated reference antibodies are LZM-009 and Atezolizumab. The conjugated antibodies tested are Compositions A and B.

FIG. 8B shows plots measuring ability of the unmodified and of conjugated antibodies to bind to human Fc gamma receptor IIIa (CD16). The figure shows mean ELISA signal on the y-axis and dosage of the human Fc gamma receptors on the x-axis. The unconjugated reference antibodies are LZM-009 and Atezolizumab. The conjugated antibodies tested are Compositions A and B.

FIG. 9 shows plots measuring ability of the unmodified and of conjugated antibodies to bind to human Fc neonatal receptor. The figure shows mean AlphaLISA signal on the y-axis and dosage of the human Fc neonatal receptor (FcRn) on the x-axis. The unconjugated reference antibodies are LZM-009 and Atezolizumab. The conjugated antibodies tested are Compositions A and B.

FIG. 10 shows plots measuring the levels PD-1 and PD-L1 surface expression on NK92 cells.

FIG. 11 shows plots measuring ability of the unconjugated IL-18 variants and corresponding IL-18 immunocytokines to stimulate the secretion of IFNgamma by NK92 cells. The figure shows mean IFNg alphaLISA signal on the y-axis and dosage of the unconjugated IL-18 variants and corresponding IL-18 immunocytokines on the x-axis. The unconjugated IL-18 variants are native IL-18 wild-type (SEQ ID NO: 1), SEQ ID NO: 30, and SEQ ID NO: 31. Corresponding IL-18 immunocytokines tested are Compositions A, B, C, and D.

FIG. 12 shows plots measuring the levels PD-1 and PD-L1 surface expression on KG-1 cells

FIG. 13 shows plots measuring the ability of the unconjugated IL-18 variants and corresponding IL-18 immunocytokines to stimulate the secretion of IFNgamma by parental PD-1^{negative} and by engineered PD-1^{positive} KG-1 cells. The figure shows mean IFNg legendplex signal on the y-axis and dosage of the unconjugated IL-18 variants and corresponding IL-18 immunocytokines on the x-axis. The unconjugated IL-18 variants are native IL-18 wild-type (SEQ ID No: 1), SEQ ID No: 30, and SEQ ID No: 31. Corresponding IL-18 immunocytokines tested are Compositions A, B, C, and D.

FIG. 14 shows plots measuring the ability of the unconjugated IL-18 variants and corresponding IL-18 immunocytokines to bind to the human IL-18 Binding Protein (IL-18BP). The figure shows mean free IL-18BP AlphaLISA signal on the y-axis and dosage of the unconjugated IL-18 variants and corresponding IL-18 immunocytokines on the x-axis. The unconjugated IL-18 variants are native IL-18 wild-type (SEQ ID NO: 1), SEQ ID NO: 30, and SEQ ID NO: 31. Corresponding IL-18 immunocytokines tested are Compositions A, B, C, and D.

FIG. 15 shows plots measuring the ability of the human IL-18 Binding Protein to inhibit the secretion of IFNgamma by NK92 cells stimulated with 2 nM of unconjugated IL-18 variants and corresponding IL-18 immunocytokines. The figure shows mean IFNg alphaLISA signal on the y-axis and dosage of the human IL-18 Binding Protein on the x-axis. The unconjugated IL-18 variants are native IL-18 wild-type (SEQ ID NO: 1), SEQ ID NO: 30, and SEQ ID NO: 31. Corresponding IL-18 immunocytokines tested are Compositions A, B, C, and D.

FIG. 16A shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows mean tumor volume on the y-axis and time on the x-axis. The immunocytokine tested in this figure is Composition A tested as a single agent at 0.3 and 1 mg/kg as two weekly i.v. injections. (n=9; mean±SEM).

FIG. 16B shows a plot describing the effect of unmodified PD-L1 antibodies and of IL-18 polypeptide conjugated PD-L1 antibody on the growth of MC38 syngeneic colon carcinoma tumors in hPD1 C57BL/6 mice. The figure shows mean tumor volume on the y-axis and time on the x-axis. The immunocytokine tested in this figure is Composition B tested as a single agent at 1 and 3 mg/kg as two weekly i.v. injections. (n=9; mean±SEM).

FIG. 17A shows a plot describing the effect of unmodified PD-1 antibodies and of IL-18 polypeptide conjugated PD-1 antibody on the body weight of MC38 syngeneic colon carcinoma tumor-bearing hPD1 C57BL/6 mice. The figure shows mean body weight change on the y-axis and time on the x-axis. The immunocytokine tested in this figure is Composition A tested as a single agent at 0.3 and 1 mg/kg as two weekly i.v. injections. (n=9; mean±SEM).

FIG. 17B shows a plot describing the effect of unmodified PD-L1 antibodies and of IL-18 polypeptide conjugated PD-L1 antibody on the body weight of MC38 syngeneic colon carcinoma tumor-bearing hPD1 C57BL/6 mice. The figure shows mean body weight change on the y-axis and time on the x-axis. The immunocytokine tested in this figure is Composition B tested as a single agent at 1 and 3 mg/kg as two weekly i.v. injections. (n=9; mean±SEM).

FIG. 18A shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows mean tumor volume on the y-axis and time on the x-axis. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM).

FIG. 18B shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows the mean tumor volume on day 17 post treatment initiation on the y-axis. The immunocytokine tested in this figure are Composition A tested as a single agent at 0.1, 0.25, and 0.5 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; One-way Anova test *** P-value<0.001, ** P-value<0.01, * P-value<0.1, ns not significant, TGI: Tumor Growth Inhibition).

FIG. 18C shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows the tumor volume of each individual animal on the y-axis and time on the x-axis. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; CR: Complete Response).

FIG. 19 shows a plot describing the effect of unmodified PD-L1 antibodies and of IL-18 polypeptide conjugated PD-1 antibody on the on the body weight of MC38 syngeneic colon carcinoma tumor-bearing hPD1 C57BL/6 mice. The figure shows mean body weight change on the y-axis and time on the x-axis. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM).

FIG. 20 shows a plot describing the effect of unmodified PD-L1 antibodies and of IL-18 polypeptide conjugated PD-1 antibody on the survival of MC38 syngeneic colon carcinoma tumor-bearing hPD1 C57BL/6 mice. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM; CR: Complete response).

FIG. 21A shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows mean tumor volume on the y-axis and time on the x-axis. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM).

FIG. 21B shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows the mean tumor volume on day 10 post treatment initiation on the y-axis. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; TGI: Tumor Growth Inhibition).

FIG. 21C shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows the tumor volume of each individual animal on the y-axis and time on the x-axis. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; CR: Complete Response).

FIG. 22 shows a plot describing the effect of unmodified PD-L1 antibodies and of IL-18 polypeptide conjugated PD-1 antibody on the body weight of B16F10 syngeneic melanoma tumor-bearing hPD1 C57BL/6 mice. The figure shows mean body weight change on the y-axis and time on the x-axis. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM).

FIG. 23 shows a plot describing the effect of unmodified PD-L1 antibodies and of IL-18 polypeptide conjugated PD-1 antibody on the survival of B16F10 syngeneic melanoma tumor-bearing hPD1 C57BL/6 mice. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM; CR: Complete response).

FIG. 24 show plots measuring ability of the unmodified and of fused anti-PD1 antibodies to bind with human PD1/CD279 ligand, with the figure showing ELISA signal on the y-axis and dosage of the biotinylated PD-1 protein on the x-axis. The unconjugated reference antibodies are Pembrolizumab/Keytruda and LZM-009 (CMP2000). The IL-18 fused antibodies tested in this figure are CMP2001 and CMP2002.

FIG. 25 shows plots measuring ability of the unmodified and of fused antibodies to bind to human PD-L1/B7-H1 ligand, with the figure showing net BioLayer interferometry shift in nanometer on the y-axis and time of incubation dosage of the biotinylated PD-1 protein on the x-axis. The reference antibodies are Pembrolizumab/Keytruda and LZM-009 (CMP2000). The IL-18 fused antibody tested in this figure is CMP2002.

FIG. 26 shows plots measuring ability of the unmodified and of fused antibodies to bind to human Fc neonatal receptor. The figure shows normalized AlphaLISA signal on the y-axis and dosage of the human Fc neonatal receptor (FcRn) on the x-axis. The reference antibodies are Pembrolizumab/Keytruda and LZM-009 (CMP2000). The IL-18 fused antibodies tested in this figure are CMP2001 and CMP2002.

FIG. 27 shows plots measuring ability of wild type IL-18 and of modified IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (OD 620 nm) on the y-axis, and dosage of the IL-18 polypeptides on the x-axis. The IL-18 polypeptides are native IL-18 wild-type (SEQ ID NO:1), SEQ ID NO: 30, CMP2001, and CMP2002.

FIG. 28 shows plots measuring ability of wild type IL-18 and of modified IL-18 polypeptides to stimulate the secretion of IFNgamma by human Peripheral Blood Mononuclear Cells (PBMCs). The figure shows mean IFNg signal on the y-axis and dosage of the IL-18 polypeptides on the x-axis. The IL-18 polypeptides are native IL-18 wild-type (SEQ ID NO:1), SEQ ID NO: 30, CMP2001 and, CMP2002.

FIG. 29 shows plots measuring the ability of wild type IL-18 and of modified IL-18 polypeptides to bind to the human IL-18 Binding Protein (IL-18BP). The figure shows free IL-18BP AlphaLISA signal on the y-axis, and dosage of IL-18 polypeptides on the x-axis. The IL-18 polypeptides are native IL-18 wild-type (SEQ ID NO:01), SEQ ID NO: 30, CMP2001 and, CMP2002.

FIG. 30 shows plots measuring the levels PD-1 surface expression on wild type NK-92 cells and on NK-92 cells transduced with human PD-1.

FIG. 31A and FIG. 31B show plots measuring the ability of of wild type IL-18 and of modified IL-18 polypeptides to stimulate the secretion of IFNgamma by parental PD-1^{negative} (grey squared symbols and dotted lines) and by engineered PD-1^{positive} NK-92 cells (black round symbols and plain lines). The figure shows mean IFNg AlphaLISA signal on the y-axis and dosage of the masked IL-18 PD-1 immunocytokines on the x-axis. The IL-18 polypeptides are native IL-18 wild-type (SEQ ID NO:1), SEQ ID NO: 30, CMP2001, CMP2002, CMP2003, CMP2004, CMP2005, CMP2006, and CMP2007.

### DETAILED DESCRIPTION

Disclosed herein are antibodies or antigen binding fragments specific for immune cell associated antigens fused to IL-18 polypeptides in fusion immunocytokines. In some instances, the fusion immunocytokines provided herein are useful as potent stimulators of one or more immune cell types (e.g., T cells, macrophages, etc.). In some embodiments, the fusion immunocytokines can act by one or more modes of action.

In some embodiments, the antibody of the fusion immunocytokine allows for targeting of the fusion immunocytokine to an immune cell. In some embodiments, the fusion immunocytokine can inhibit an activity of the immune cell associated antigen (e.g., inhibiting a checkpoint interaction such as a PD-1/PD-L1 interaction) through binding to the immune cell associated antigen. In some embodiments, the fusion immunocytokines induce IFNY production in immune cells (e.g., T cells or NK cells). The antibody or antigen binding fragment-IL-18 fusion immunocytokines of the disclosure can have synergistic efficacy and improved tolerability by a subject. In some embodiments, the antibody or antigen binding fragment-IL-18 fusion immunocytokines can significantly reduce the therapeutic dose of the antibody or antigen binding fragment, the IL-18 polypeptide, or both for a subject with a disease, such as a cancer, as compared to a treatment with one or both entities individually or in combination. In some embodiments, the fusion immunocytokines provided herein are associated with fewer side effects than administration of one or both entities individually or in combination, potentially due to the targeting nature of the antibodies for an immune cell.

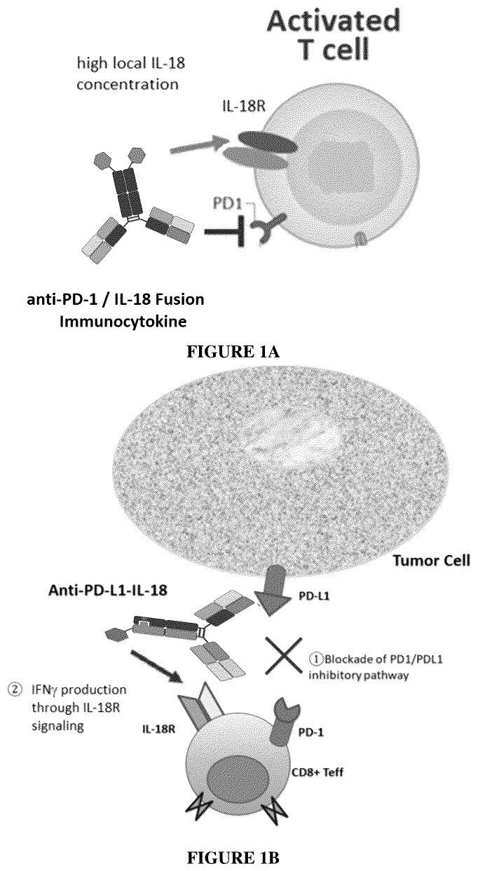

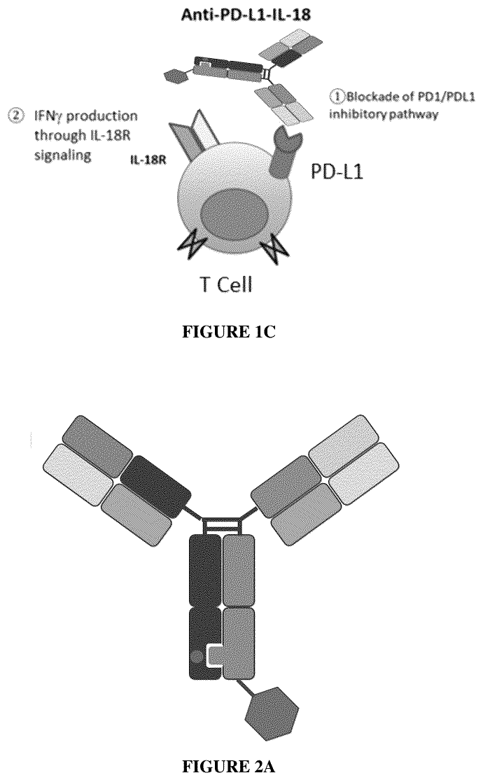

An exemplary, non-limiting mechanism of action of a fusion immunocytokine provided herein is shown in FIG. 1A. In the exemplary embodiment, the fusion immunocytokine comprises an anti-PD-1 antibody as the antibody or antigen binding fragment of the fusion immunocytokine. In this embodiment, the anti-PD-1 antibody portion of the fusion immunocytokine selectively binds to PD-1 present on the surface of an activated T cell (e.g., a CD8+T cell). This binding prevents the checkpoint interaction of PD-1 and PD-L1/2, thus preventing attenuation of activity of the T cell. Concomitantly, the IL-18 portion of the fusion immunocytokine, which is effectively in a high local concentration near the T-cell due to the linkage, further activates the T cell through IL-18R signaling. While the exemplary embodiment shows the mechanism of action of an anti-PD-1 antibody, antibodies or antigen binding fragments specific for other immune antigens provided herein can function according to a similar mechanism.

Another exemplary, non-limiting mechanism of action of an anti-PD-L1 antibody/IL-18 fusion immunocytokine is shown in FIG. 1B. In the exemplary embodiment, the anti-PD-L1 antibody portion of the fusion immunocytokine binds to PD-L1 expressed on the surface of a tumor cell. When a T cell comes into contact with the tumor cell, an interaction between PD-1 on the T cell and PD-L1 on the cell is blocked, preventing attenuation of the activity of the T cell. Additionally, the IL-18 portion of the fusion immunocytokine is free to signal through IL-18R on the immune cell, thereby inducing production of IFNγ and further activation of the immune cell. Though this exemplary embodiment is demonstrated for PD-L1, other immune antigens provided herein in fusion immunocytokines may display similar mechanisms of action.

In a similar manner, FIG. 1C depicts an alternative non-limiting mechanism of action of an anti-PD-L1 antibody/IL-18 fusion immunocytokine as described herein acting upon a T cell expression PD-L1 and IL-18R. Analogously to the mechanism depicted in FIG. 1A, the antibody portion of the fusion immunocytokine binds to the PD-L1 resent on the surface of the T cell. Such binding accomplishes two tasks: 1) the targeting of the IL-18 polypeptide to the T cell, and 2) blocking of the interaction of PD-L1 on the surface of the cell with PD-1 elsewhere in the environment, thereby blocking the immune checkpoint inhibitory pathway.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Immune Antigen Specific Antibodies Linked to IL-18 Polypeptides in Fusion Immunocytokines

Provided herein are antibodies and antigen binding fragments which binds to immune cell associated antigens fused to IL-18 polypeptides as fusion immunocytokines. The fusion immunocytokines provided herein are effective for simultaneously delivering the IL-18 polypeptide and the antibody or antigen binding fragment to a target cell, such as an immune cell. This simultaneous delivery of both agents to the same cell has numerous benefits, including improved IL-18 polypeptide selectivity, enhanced therapeutic potential of the IL-18 polypeptide, and minimized risk of side effects from administering IL-18 therapies. In some embodiments, the fusion immunocytokine acts through multiple modes of action, including without limitation disrupting an activity of the immune cell associated antigen (e.g., immune checkpoint evasion) and/or enhanced activation of immune cells in or around a tumor microenvironment (e.g., due to targeting effects).

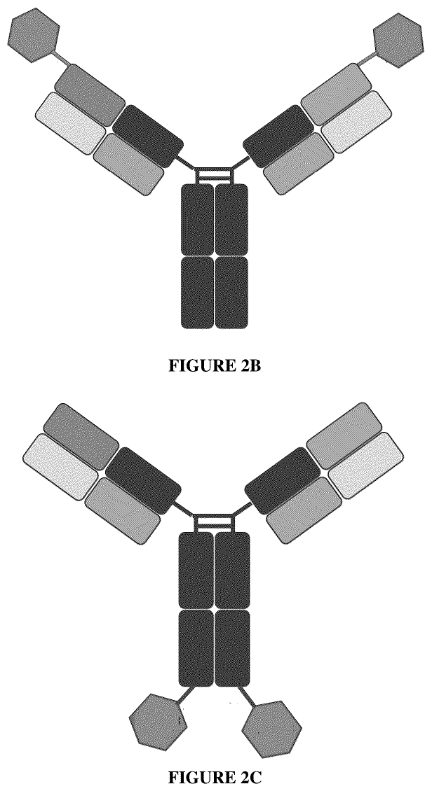

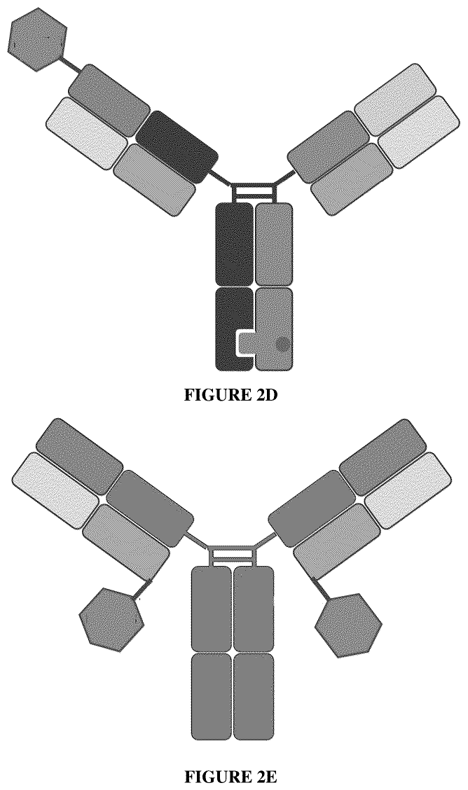

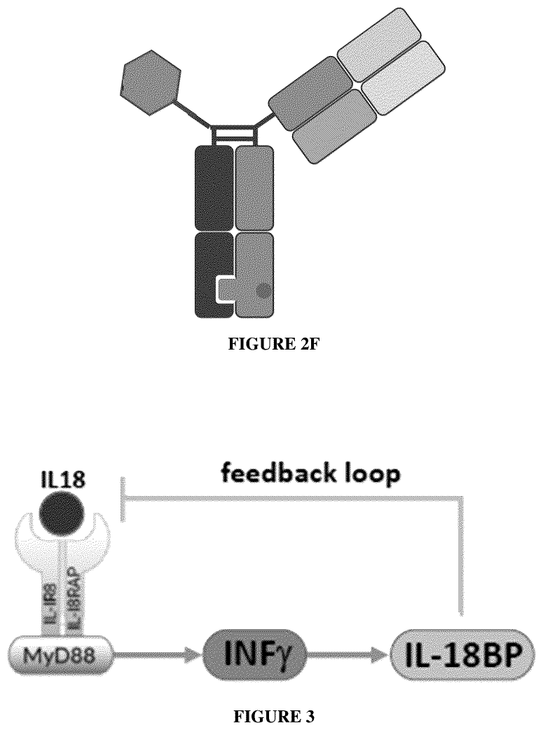

The fusion immunocytokines provided herein are fused to the antibody or antigen binding fragments. The IL-18 polypeptides can be fused at any desired portion of the antibody or antigen binding fragment which maintains the activity of both portions of the fusion immunocytokine, though certain architectures of the fusion immunocytokines may offer certain advantages as described in more detail herein. Exemplary orientations and architectures include those described in FIGS. 2A-2F, and can include the IL-18 polypeptide fused via either its N-or C-terminus to the N- or C-terminus of any polypeptide of the antibody or antigen binding fragment, or a portion thereof (e.g., an Fc domain which pairs with an opposing Fc domain which comprises an antigen binding domain, such as that depicted in FIG. 2F).

The IL-18 containing fusion immunocytokines described herein can be assembled according to a wide variety of architectures for preparing fusion proteins with antibodies. A non-limiting exemplary set of such architectures are provide in FIGS. 2A-2F. In each figure, the hexagon shape represents the IL-18 polypeptide.

FIG. 2A shows a fusion immunocytokine with a single IL-18 polypeptide fused via its N-terminus to the C-terminal end of one of the heavy chains of the antibody. The IL-18 polypeptide is optionally fused through a peptide linker as otherwise described herein. In order to manufacture such a fusion immunocytokine with only a single IL-18 polypeptide attached, it can be desirable to incorporate modifications into one or both of the Fc domains of the heavy chain(s) of the antibody in order to facilitate the joining of the two different polypeptides into the final desired fusion immunocytokine format. Such modifications and strategies are known in the art for the manufacture of bispecific antibodies. For example, in some embodiments, such antibodies with asymmetric arms of the antibody structure incorporate knob-into-hole technology (e.g., as described in, for example, U.S. Pat. No. 8,216,805) or modifications to one arm to abrogate binding to protein A to facilitate purification of the desired bispecific (e.g., the so-called “RF” mutation described in, for example, U.S. Pat. No. 11,168,111). In FIG. 2A, the “knob” mutations (e.g., S354C, T366W (EU numbering)) are incorporated into the Fc domain to which the IL-18 polypeptide is fused, depicted in the figure as the protrusion in the CH3 domain, and the hole mutations (e.g., Y349C, T366S, L368A, Y407V (EU numbering)) are incorporated into the opposing Fc domain. The alternative approach where the hole mutations are incorporated into the Fc domain to which the IL-18 polypeptide is fused is also contemplated. FIG. 2A also depicts an RF mutation (H435R, Y436F (EU numbering)) incorporated into the Fc domain which is not fused to the IL-18 polypeptide, though the alternative configuration is also contemplated. Such strategies are applicable to all of the asymmetric fusion immunocytokine architectures provided herein. Other modifications to antibodies known in the art to impart one or more desired properties to the antibodies without compromising the antigen binding ability of the antibody portion can also be incorporated into the fusion immunocytokine of the format depicted in FIG. 2A or into any other fusion immunocytokine architecture provided herein (e.g., half-life extension modifications, such as the “YTE” set of substitutions known in the art).

FIG. 2B shows a fusion immunocytokine with two IL-18 polypeptides fused to the antibody, both IL-18 polypeptides fused via their respective C-terminuses to the N-terminal ends of the heavy chains of the antibody (i.e., the N-terminal end of the heavy chain variable region), optionally by a peptide linker. The peptide linker is preferably the same for both portions of the molecule. Additional modifications to the Fc domain(s) of this fusion immunocytokine architecture are also contemplated.

FIG. 2C shows a fusion immunocytokine with two IL-18 polypeptides fused to the antibody, both IL-18 polypeptides fused via their respective N-terminuses to the C-terminal ends of the heavy chains of the antibody (i.e., the C-terminal end of the CH3 domain), optionally by a peptide linker which is preferably the same for both portions of the molecule. Modifications to the Fc domain(s) of this fusion immunocytokine architecture are also contemplated. Other modifications described herein to the Fc domains of the two portions of the molecule can also be incorporated.

FIG. 2D shows a fusion immunocytokine with a single IL-18 polypeptide fused via its C-terminus to the N-terminal end of one of the heavy chains of the antibody (i.e., the—terminal end of the heavy chain variable region, optionally by a peptide linker which is preferably the same for both portions of the molecule. In FIG. 2D, a hole modification (e.g., Y349C, T366S, L368A, Y407V (EU numbering)) is shown incorporated into Fc domain which is part of the heavy chain to which the IL-18 polypeptide is fused, with the corresponding Fc domain containing the knob mutations (e.g., S354C, T366W (EU numbering)), though the opposite configuration is also contemplated. The molecule is also depicted as containing an RF mutation (H435R, Y436F (EU numbering)) in the heavy chain to which the IL-18 polypeptide is not fused, though the opposite configuration is similarly contemplated. Other modifications described herein to the Fc domains of the two portions of the molecule can also be incorporated.

FIG. 2E shows a fusion immunocytokine with two IL-18 polypeptides fused to the antibody, both IL-18 polypeptides fused via their respective N-terminuses to the C-terminal ends of the light chains of the antibody (i.e., the C-terminal end of the light chain constant region), optionally by peptide linkers. The IL-18 polypeptides could also be fused to the N-terminal ends of the light chains of the antibody (i.e., to the N-terminal end of the light chain variable domain via the C-terminus of the IL-18 polypeptide, optionally through a peptide linker). Other modifications described herein to the Fc domains of the two portions of the molecule can also be incorporated.

FIG. 2F shows a fusion immunocytokine with a single IL-18 polypeptide fused via its C-terminus to an Fc domain (i.e., fused to the N-terminus of a CH2 domain) which is paired with a monovalent antibody domain, optionally through a peptide linker. Alternatively, the IL-18 polypeptide can be fused to the C-terminus of the CH3 domain (i.e., by the N-terminus of the IL-18 polypeptide). As with the other asymmetric antibodies described herein, the Fc domain fused to the IL-18 polypeptide or the Fc domain containing the antigen binding domain of the antibody (or both) can comprises one or more modifications to facilitate the joining of the two different portions of the molecule (e.g., knob-into-hole technology, RF modifications, etc.). The fusion immunocytokine depicted in the figure includes hole mutations (e.g., Y349C, T366S, L368A, Y407V (EU numbering)) in the Fc domain to which the IL-18 polypeptide is fused and corresponding knob (e.g., S354C, T366W (EU numbering)) mutations in the opposing Fc domain, with the opposite orientation also contemplated. Other modifications described herein to the Fc domains of the two portions of the molecule can also be incorporated. The molecule is also depicted as containing an RF mutation (H435R, Y436F (EU numbering)) in the Fc domain to which the IL-18 polypeptide is not fused, though the opposite configuration is similarly contemplated. Other modifications described herein to the Fc domains of the two portions of the molecule can also be incorporated.

In some embodiments, the IL-18 polypeptides are fused to the antibodies or antigen binding fragments via peptide linkers. Such peptide linkers can, in certain embodiments, allow for the IL-18 polypeptide activity to be better maintained compared to a corresponding fusion immunocytokine which lacks a peptide linker, though in some embodiments a peptide linker is not required. In some embodiments, the peptide linker is desirably a flexible peptide linker (e.g., comprised partially or entirely of glycine and/or serine residues) in order to allow the IL-18 polypeptide to maintain a desirable orientation relative to the antibody or antigen binding fragment portion of the fusion immunocytokine in order to enhance activity of the fusion immunocytokine.

In some embodiments, the IL-18 polypeptide is modified relative to human wild type IL-18 (SEQ ID NO: 1). In some embodiments, these modifications comprises one or more amino acid substitutions which desirably alter the properties of the IL-18 polypeptide. In some embodiments, the IL-18 polypeptide has a reduced ability to be inhibited by IL-18 binding protein. In some embodiments, the IL-18 polypeptide has an enhanced ability to activate or signal through the IL-18 receptor, such as by exhibiting enhanced binding to the IL-18 receptor or through another mechanism.

In one aspect, provided herein, is a fusion immunocytokine, comprising: an IL-18 polypeptide fused to an antibody or an antigen binding fragment thereof specific for an immune cell associated antigen, optionally through a peptide linker.

### Immune Cell Specific Antibodies

In some embodiments, an antibody or an antigen binding fragment of the fusion immunocytokine specifically binds to an immune cell associated antigen. An immune cell associated antigen provided herein is an antigen which associated with expression on immune cells or associated with activity of immune cells (e.g., an antigen associated with immune cell activation or deactivation), or both. In some embodiments, the immune cell associated antigen is expressed at a level of at least 25% greater, at least 30% greater, at least 40% greater, at least 50% greater, at least 60% greater, at least 70% greater, at least 80% greater, at least 90% greater, or at least 100% greater in the immune cell than another cell. In some embodiments, the immune cell associated antigen is expressed at a level of at least 2-fold greater, at least 4-fold greater, at least 6-fold greater, at least 8-fold greater, or at least 10-fold greater in the immune cell than another cell. Non-limiting examples of immune cell associated antigens include 4-IBB, CD3, CCR8, CD8A, CD8B, CD16A, CD28, CD80, CD86, CD96, CD226, CTLA-4, D40, GITR, ICOS, LAG-3, MHCI, MHCII, NKG2A, NKG2D, NKp30, NKp44, NKp46, OX40, PD-1, PD-L1, PD-L2, SIRPA, TCR, TIGIT, TIM-3, and VISTA.

An antibody selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. In some embodiments, an antibody or an antigen binding fragment of the disclosure can inhibit the action/activity of the substance to which it binds. In some embodiments, an antibody or antigen binding fragment of the disclosure can agonize the action/activity of the substance to which it binds (e.g., an immune cell agonist antibody or antigen binding fragment such as one specific for CD16A, NKG2D, NKp30, or other targets).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light-and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a CH3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a CH3 domain can be from an immunoglobulin selected from the group consisting of an IgG, an IgA, an IgD, an IgE, and an IgM, or is derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some instances, an antibody or antigen binding fragment comprises an IgG2 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“K” or “K”) or lambda (“2”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (Kabat et al., Sequences of Proteins of Immunological Interest, 5th Ed., 1991, National Institutes of Health, Bethesda Md., pages 647-669; hereafter “Kabat”); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a B-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat et al., Id. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196:901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Id. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (V_{H} CDR1), heavy chain second complementarity determining region (V_{H} CDR2), heavy chain third complementarity determining region (VH CDR3), light chain first complementarity determining region (V_{L} CDR1), light chain second complementarity determining region (V_{L} CDR2), and light chain third complementarity determining region (V_{L} CDR3).

The term “Fc domain” is used to define a C-terminal region of an immunoglobulin heavy chain. The “Fc domain” may be a native sequence Fc domain or a variant Fc domain. Although the boundaries of the Fc domain of an immunoglobulin heavy chain might vary, the human IgG heavy chain Fc domain is generally defined to stretch from an amino acid residue at position Cys226, or from Pro230, to the carboxyl-terminus thereof. The numbering of the residues in the Fc domain is that of the EU index as in Kabat. The Fc domain of an immunoglobulin generally comprises two constant domains, CH2 and CH3.

“Antibodies” useful in the present disclosure encompass, but are not limited to, monoclonal antibodies, polyclonal antibodies, chimeric antibodies, bispecific antibodies, grafted antibodies, multispecific antibodies, heteroconjugate antibodies, humanized antibodies, human antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc domains modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro). A human or humanized antibody can comprise modifications to the antibody sequence or structure which are known in the art, such as half-life extension modifications (e.g., YTE substitutions), or other modifications.

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the co-expression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′) 2, a bispecificF(ab′) 2, a trispecific F(ab′) 2, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/V_{L} described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/V_{L} described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides). In certain embodiments of the invention, a full length antibody (e.g., an antigen binding fragment and an Fc domain) are preferred.

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 224), (GGS)ₙ (SEQ ID NO: 225), (GGGS)ₙ (SEQ ID NO: 226), (GGSG)ₙ (SEQ ID NO: 227), or (GGSGG)ₙ (SEQ ID NO: 228), (GGGGS)ₙ (SEQ ID NO: 229), wherein n is 1, 2, 3, 4, 5, 6, 7, 8, 9, or 10. For example, a linking peptide can be (GGGGS) 3 (SEQ ID NO: 230) or (GGGGS) 4 (SEQ ID NO: 231). In some embodiments, a linking peptide bridges approximately 3.5 nm between the carboxy terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as Kp. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quatravalent antibody or antigen binding fragment.

An antibody or antigen binding fragment described herein can be isolated, purified, recombinant, or synthetic.

It is contemplated that generic or biosimilar versions of the named antibodies herein which share the same amino acid sequence as the indicated antibodies are also encompassed when the name of the antibody is used.

The antibodies described herein may be made by any suitable method. Antibodies can often be produced in large quantities, particularly when utilizing high level expression vectors.

In one embodiment, an antibody or an antigen binding fragment of the disclosure comprises a fusion protein or a peptide immunotherapeutic agent.

### Immune Cell Associated Antigen Specific Antibodies

In some embodiments, the antibody or antigen binding fragment thereof is specific for an immune cell associated antigen. In some embodiments, the immune cell associated antigen is associated with an immune cell subtype (e.g., lymphocyte, neutrophil, macrophage, etc.). In some embodiments, the immune cell associated antigen is associated with a T cell, a monocyte, and/or a natural killer (NK) cell. In embodiments, the immune cell antigen is associated with a T cell. In some embodiments, the immune cell antigen is associated with an effector T cell, a cytotoxic T cell, a helper T cell, a regulatory T cell, and/or a memory T cell.

In some embodiments, the immune cell associated antigen is an immune checkpoint molecule. In some embodiments, the immune cell associated antigen is a costimulatory antigen. In some embodiments, the immune cell associated antigen is a macrophage cell surface antigen. In some embodiments, the immune cell associated antigen is an NK cell surface antigen. In some embodiments, the immune cell associated antigen is a T cell surface antigen (e.g., CD8A, CD8B).

In some embodiments, the immune cell associated antigen is 4-IBB, B7-H3, B7-H4, BTLA, CD3, CCR8, CD8A, CD8B, CD16A, CD27, CD28, CD33, CD38, CD39, CD40, CD47, CD70, CD80, CD86, CD96, CD163, CLEC-1, CLEVER-1, CTLA-4, D40, GITR, ICOS, ILT2/3/4, LAG-3, MHCI, MHCII, NKG2A, NKG2D, NKp30, NKp44, NKp46, OX40, PD-1, PD-L1, PD-L2, PSGL-1, SIGLEC-9, SIGLEC-15, SIRP-α, TCR,TIGIT, TIM-3, VISTA, or VSIG4. In some embodiments, the immune cell associated antigen is PD-1. In some embodiments, the immune cell associated antigen is CCR8, CD8A, CD8B, CD16A, CD96, CD226, CTLA-4, ICOS, LAG-3, NKG2A, NKG2D, NKp30, NKp44, NKp46, PD-1, PD-L1, TIGIT, or TIM-3.

In some embodiments, the immune cell associated antigen is 4-1BB. In some embodiments, the immune cell associated antigen is B7-H3. In some embodiments, the immune cell associated antigen is B7-H4. In some embodiments, the immune cell associated antigen is BTLA. In some embodiments, the immune cell associated antigen is CD3. In some embodiments, the immune cell associated antigen is CCR8. In some embodiments, the immune cell associated antigen is CD8A. In some embodiments, the immune cell associated antigen is CD8B. In some embodiments, the immune cell associated antigen is CD16A. In some embodiments, the immune cell associated antigen is CD27. In some embodiments, the immune cell associated antigen is CD33. In some embodiments, the immune cell associated antigen is CD38. In some embodiments, the immune cell associated antigen is CD39. In some embodiments, the immune cell associated antigen is CD40. In some embodiments, the immune cell associated antigen is CD47. In some embodiments, the immune cell associated antigen is CD80. In some embodiments, the immune cell associated antigen is CD86. In some embodiments, the immune cell associated antigen is CD96. In some embodiments, the immune cell associated antigen is CD163. In some embodiments, the immune cell associated antigen is CLEC-1. In some embodiments, the immune cell associated antigen is CLEVER-1. In some embodiments, the immune cell associated antigen is CTLA4. In some embodiments, the immune cell associated antigen is D40. In some embodiments, the immune cell associated antigen is GITR. In some embodiments, the immune cell associated antigen is ICOS. In some embodiments, the immune cell associated antigen is ILT2/3/4. In some embodiments, the immune cell associated antigen is LAG-3. In some embodiments, the immune cell associated antigen is MHCI. In some embodiments, the immune cell associated antigen is MHCII. In some embodiments, the immune cell associated antigen is NKG2A. In some embodiments, the immune cell associated antigen is NKp30. In some embodiments, the immune cell associated antigen is NKp44. In some embodiments, the immune cell associated antigen is NKp46. In some embodiments, the immune cell associated antigen is OX40. In some embodiments, the immune cell associated antigen is PD-1. In some embodiments, the immune cell associated antigen is PD-L1. In some embodiments, the immune cell associated antigen is PD-L2. In some embodiments, the immune cell associated antigen is PSGL-1. In some embodiments, the immune cell associated antigen is SIGLEC-9. In some embodiments, the immune cell associated antigen is SIGLEC-15. In some embodiments, the immune cell associated antigen is SIRP . . . . In some embodiments, the immune cell associated antigen is TCR. In some embodiments, the immune cell associated antigen is TIGIT. In some embodiments, the immune cell associated antigen is TIM-3. In some embodiments, the immune cell associated antigen is VISTA. In some embodiments, the immune cell associated antigen is VSIG4.

In some embodiments, the antibody or antigen binding fragment thereof is an anti-PD-1 antibody or antigen binding fragment. Programmed cell death protein 1 (also known as PD-1 and CD279), is a cell surface receptor that plays an role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is

(SEQ ID NO: 331)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In one embodiment, an anti-PD-1 antibody or an anti-PD-1 antigen binding fragment of the disclosure comprises a combination of a heavy chain variable region (VH) and a light chain variable region (VL) described herein. In another embodiment, an anti-PD-1 antibody or an anti-PD-1 antigen binding fragment of the disclosure comprises a combination of complementarity determining regions (V_{H} CDR1, V_{H} CDR2, V_{H} CDR3, V_{L} CDR1, VL CDR2, and V_{L} CDR3) described herein, or which are contained in a heavy chain variable region and light chain variable region described herein. In one embodiment, an anti-PD-1 antibody or an anti-PD-1 antigen binding fragment of the disclosure comprises a modified Tislelizumab, Baizean, OKVO411B3N, BGB-A317, hu317-1/lgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QV_{L}057INT, H4H7798N, REGN-2810, SAR-439684, Avelumab, BAVENCIO®, 451238, KXG2PJ551I, MSB-0010682, MSB-0010718C, PF-06834635, Durvalumab, IMFINZI®, 28X28X9OKV, MEDI-4736, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, POGVQ9A4S5, TSR-042, WBP-285, Serplulimab, HLX-10, CS-1003, Retifanlimab, 2Y3T5IF01Z, INCMGA-00012, INCMGA-0012, MGA-012, Sasanlimab, LZZOIC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDI0680, or AMP-514.

In some embodiments, an anti-PD-1 antibody or an anti-PD-1 antigen binding fragment of the disclosure comprises a Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab. In one embodiment, an anti-PD-1 antibody or an anti-PD-1 antigen binding fragment of the disclosure comprises a modified Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab, or a modified version of any of these, or a V_{H} and V_{L} of any of these, or CDRs of any of these.

In some embodiments, the anti-PD-1 antibody or antigen binding fragment comprises Nivolumab, Pembrolizumab, LZM-009, Dostarlimab, Sintilimab, Spartalizumab, Tislelizumab, or Cemiplimab, a modified version of any of these, or a V_{H} and V_{L} of any of these, or CDRs of any of these. In some embodiment, the anti-PD-1 antibody or antigen binding fragment is Dostarlimab, Sintilimab, Spartalizumab, or Tislelizumab, a modified version of any of these, or a V_{H} and V_{L} of any of these, or CDRs of any of these. In some embodiments, the anti-PD-1 polypeptide comprises Nivolumab, Pembrolizumab, LZM-009, or Cemiplimab, a modified version of any of these, or a V_{H} and V_{L} of any of these, or CDRs of any of these.

In some embodiments, the anti-PD-1 antibody comprises Pembrolizumab. In some embodiments, the anti-PD-1 antibody comprises modified Pembrolizumab. In some embodiments, the anti-PD-1 antibody comprises the V_{H} and V_{L} of Pembrolizumab (e.g., as shown in Table 1 below). In some embodiments, the anti-PD-1 antibody comprises the CDRs of Pembrolizumab.

In some embodiments, the anti-PD-1 antibody comprises LZM-009. In some embodiments, the anti-PD-1 antibody comprises modified LZM-009. In some embodiments, the anti-PD-1 antibody comprises the V_{H} and V_{L} of LZM-009 (e.g., as shown in Table 1 below). In some embodiments, the anti-PD-1 antibody comprises the CDRs of LZM-009.

TABLE 1 provides the sequences of exemplary anti-PD-1 antibodies and anti-PD-1 antigen binding fragments that can be modified to prepare anti-PD-1 fusion immunocytokines. TABLE 1 also shows provides combinations of CDRs that can be utilized in a modified anti-PD-1 fusion immunocytokine. Reference to an anti-PD-1 antibody herein may alternatively refer to an anti-PD-1 antigen binding fragment.

In some instances, the SEQ ID NOs listed in Table 1 contain full-length heavy or light chains of the indicated antibodies with the V_{H} or V_{L} respectively indicated in bold. Where there is a reference herein to a V_{H} or V_{L} of a SEQ ID NO in Table 1 which contains a full-length heavy or light chain, it is intended to reference the bolded portion of the sequence. For example, reference to “a V_{H} having an amino acid sequence shown in SEQ ID NO: 332” refers to the bolded portion of SEQ ID NO: 332 in Table 1.

An anti-PD-1 antibody or an anti-PD-1 antigen binding fragment can comprise a VH having an amino acid sequence shown in any one of SEQ ID NOS: 332, 334, 336, 338, 340, 346, 348, 350, 352, 354, 356, 358, 360, 362, 364, 366, 368, 370, 372, 374, 376, and 378. An anti-PD-1 antibody or an anti-PD-1 antigen binding fragment can comprise a V_{L} having an amino acid sequence shown in any one of SEQ ID NOS: 333, 335, 337, 339, 341, 347, 349, 351, 353, 355, 357, 359, 361, 363, 365, 367, 369, 371, 373, 375, 377, and 379.

An anti-PD-1 antibody or an anti-PD-1 antigen binding fragment can comprise a heavy chain or V_{H} having an amino acid sequence of any one of SEQ ID NOS: 332, 334, 336, 338, 340, 346, 348, 350, 352, 354, 356, 358, 360, 362, 364, 366, 368, 370, 372, 374, 376, and 378, or a portion corresponding to a V_{H} thereof. An anti-PD-1 antibody or an anti-PD-1 antigen binding fragment can comprise a light chain or V_{L} having an amino acid sequence of any one of SEQ ID NOS: 333, 335, 337, 339, 341, 347, 349, 351, 353, 355, 357, 359, 361, 363, 365, 367, 369, 371, 373, 375, 377, and 379, or a portion corresponding to a V_{L} thereof.

In one instance, an anti-PD-1 antibody or an anti-PD-1 antigen binding fragment comprises a V_{H} having an amino acid sequence shown in SEQ ID NO: 332, and a V_{L} having an amino acid sequence shown in SEQ ID NO: 333. In another instance, an anti-PD-1 antibody or an anti-PD-1 antigen binding fragment comprises a V_{H} having an amino acid sequence shown in SEQ ID NO: 334, and a V_{L} having an amino acid sequence shown in SEQ ID NO: 335. In another instance, an anti-PD-1 antibody or an anti-PD-1 antigen binding fragment comprises a V_{H} having an amino acid sequence shown in SEQ ID NO: 336, and a V_{L} having an amino acid sequence shown in SEQ ID NO: 337. In another instance, an anti-PD-1 antibody or an anti-PD-1 antigen binding fragment comprises a V_{H} having an amino acid sequence shown in SEQ ID NO: 338, and a V_{L} having an amino acid sequence shown in SEQ ID NO: 339. In another instance, an anti-PD-1 antibody or an anti-PD-1 antigen binding fragment comprises a VH having an amino acid sequence shown in SEQ ID NO: 340, and a VL having an amino acid sequence shown in SEQ ID NO: 341. In another instance, an anti-PD-1 antibody or an anti-PD-1 antigen binding fragment comprises a VH having an amino acid sequence shown in SEQ ID NO: 346, and a VL having an amino acid sequence shown in SEQ ID NO: 347. In another instance, an anti-PD-1 antibody or an anti-PD-1 antigen binding fragment comprises a VH having an amino acid sequence shown in SEQ ID NO: 348, and a VL having an amino acid sequence shown in SEQ ID NO: 349. In another instance, an anti-PD-1 antibody or an anti-PD-1 antigen binding fragment comprises a VH having an amino acid sequence shown in SEQ ID NO: 350, and a VL having an amino acid sequence shown in SEQ ID NO: 351. In another instance, an anti-PD-1 antibody or an anti-PD-1 antigen binding fragment comprises a VH having an amino acid sequence shown in SEQ ID NO: 352, and a VL having an amino acid sequence shown in SEQ ID NO: 353. In another instance, an anti-PD-1 antibody or an anti-PD-1 antigen binding fragment comprises a VH having an amino acid sequence shown in SEQ ID NO: 354, and a VL having an amino acid sequence shown in SEQ ID NO: 355. In another instance, an anti-PD-1 antibody or an anti-PD-1 antigen binding fragment comprises a VH having an amino acid sequence shown in SEQ ID NO: 356, and a VL having an amino acid sequence shown in SEQ ID NO: 357. In another instance, an anti-PD-1 antibody or an anti-PD-1 antigen binding fragment comprises a VH having an amino acid sequence shown in SEQ ID NO: 358, and a VL having an amino acid sequence shown in SEQ ID NO: 359. In another instance, an anti-PD-1 antibody or an anti-PD-1 antigen binding fragment comprises a VH having an amino acid sequence shown in SEQ ID NO: 360, and a VL having an amino acid sequence shown in SEQ ID NO: 361. In another instance, an anti-PD-1 antibody or an anti-PD-1 antigen binding fragment comprises a VH having an amino acid sequence shown in SEQ ID NO: 362, and a VL having an amino acid sequence shown in SEQ ID NO: 363. In another instance, an anti-PD-1 antibody or an anti-PD-1 antigen binding fragment comprises a VH having an amino acid sequence shown in SEQ ID NO: 364, and a VL having an amino acid sequence shown in SEQ ID NO: 365. In another instance, an anti-PD-1 antibody or an anti-PD-1 antigen binding fragment comprises a VH having an amino acid sequence shown in SEQ ID NO: 366, and a VL having an amino acid sequence shown in SEQ ID NO: 367. In another instance, an anti-PD-1 antibody or an anti-PD-1 antigen binding fragment comprises a VH having an amino acid sequence shown in SEQ ID NO: 368, and a VL having an amino acid sequence shown in SEQ ID NO: 369. In another instance, an anti-PD-1 antibody or an anti-PD-1 antigen binding fragment comprises a VH having an amino acid sequence shown in SEQ ID NO: 370, and a VL having an amino acid sequence shown in SEQ ID NO: 371. In another instance, an anti-PD-1 antibody or an anti-PD-1 antigen binding fragment comprises a VH having an amino acid sequence shown in SEQ ID NO: 372, and a VL having an amino acid sequence shown in SEQ ID NO: 373. In another instance, an anti-PD-1 antibody or an anti-PD-1 antigen binding fragment comprises a VH having an amino acid sequence shown in SEQ ID NO: 374, and a VL having an amino acid sequence shown in SEQ ID NO: 375. In another instance, an anti-PD-1 antibody or an anti-PD-1 antigen binding fragment comprises a VH having an amino acid sequence shown in SEQ ID NO: 376, and a VL having an amino acid sequence shown in SEQ ID NO: 377. In another instance, an anti-PD-1 antibody or an anti-PD-1 antigen binding fragment comprises a VH having an amino acid sequence shown in SEQ ID NO: 378, and a VL having an amino acid sequence shown in SEQ ID NO: 379.

In one instance, an anti-PD-1 antibody or an anti-PD-1 antigen binding fragment comprises a VH CDR1 having an amino acid sequence of SEQ ID NO: 380, a VH CDR2 having an amino acid sequence of SEQ ID NO: 381, a VH CDR3 having an amino acid sequence of SEQ ID NO: 382, VL CDR1 having an amino acid sequence of SEQ ID NO: 383, a VL CDR2 having an amino acid sequence of SEQ ID NO: 384, and a VL CDR3 having an amino acid sequence of SEQ ID NO: 385. In one instance, an anti-PD-1 antibody or an anti-PD-1 antigen binding fragment comprises a VH CDR1 having an amino acid sequence of SEQ ID NO: 386, a VH CDR2 having an amino acid sequence of SEQ ID NO: 387, a VH CDR3 having an amino acid sequence of SEQ ID NO: 388, VL CDR1 having an amino acid sequence of SEQ ID NO: 389, a VL CDR2 having an amino acid sequence of SEQ ID NO: 390, and a VL CDR3 having an amino acid sequence of SEQ ID NO: 391. In one instance, an anti-PD-1 antibody or an anti-PD-1 antigen binding fragment comprises a VH CDR1 having an amino acid sequence of SEQ ID NO: 392, a VH CDR2 having an amino acid sequence of SEQ ID NO: 393, a VH CDR3 having an amino acid sequence of SEQ ID NO: 394, VL CDR1 having an amino acid sequence of SEQ ID NO: 395, a VL CDR2 having an amino acid sequence of SEQ ID NO: 396, and a VL CDR3 having an amino acid sequence of SEQ ID NO: 397. In one instance, an anti-PD-1 antibody or an anti-PD-1 antigen binding fragment comprises a VH CDR1 having an amino acid sequence of SEQ ID NO: 398, a VH CDR2 having an amino acid sequence of SEQ ID NO: 399, a VH CDR3 having an amino acid sequence of SEQ ID NO: 400, VL CDR1 having an amino acid sequence of SEQ ID NO: 401, a VL CDR2 having an amino acid sequence of SEQ ID NO: 402, and a VL CDR3 having an amino acid sequence of SEQ ID NO: 403. In one instance, an anti-PD-1 antibody or an anti-PD-1 antigen binding fragment comprises a VH CDR1 having an amino acid sequence of SEQ ID NO: 404, a VH CDR2 having an amino acid sequence of SEQ ID NO: 405, a VH CDR3 having an amino acid sequence of SEQ ID NO: 406, VL CDR1 having an amino acid sequence of SEQ ID NO: 407, a VL CDR2 having an amino acid sequence of SEQ ID NO: 408, and a VL CDR3 having an amino acid sequence of SEQ ID NO: 409.

In some embodiments, the antibody or antigen binding fragment thereof is an anti-PD-L1 antibody or antigen binding fragment. Programmed death-ligand 1 (PD-L1) is a ligand for an immunosuppressive receptor “programmed death receptor 1 (PD-1)” that is predominantly expressed in activated T and B cells, which can negatively regulate antigen receptor signaling. The ligands (PD-L1 and PD-L2) for PD-1 may be constitutively expressed or may be derived into a number of cell types, including non-hematopoietic cell tissues and various tumor types. PD-L1 is expressed in B cells, T cells, bone marrow cells and dendritic cells (DCs), but also on non-lymphatic organs such as peripheral cells, pseudo-vascular endothelial cells and heart, lungs, etc. A non-limiting, exemplary, human PD-L1 amino acid sequence is

(SEQ ID NO: 330)
MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTTNEIFYCTFRRLDPEENHTAELV
IPELPLAHPPNERTHLVILGAILLCLGVALTFIFRLRKGRMMDVK
KCGIQDTNSKKQSDTHLEET

In one embodiment, an anti-PD-L1 antibody or an anti-PD-L1 antigen binding fragment of the disclosure comprises a combination of a heavy chain variable region (VH) and a light chain variable region (VL) described herein. In another embodiment, an anti-PD-L1 antibody or an anti-PD-L1 antigen binding fragment of the disclosure comprises a combination of complementarity determining regions (VH CDR1, VH CDR2, VH CDR3, VL CDR1, VL CDR2, and VL CDR3) described herein, or of any antibody as described herein. In one embodiment, an anti-PD-L1 antibody or an anti-PD-L1 antigen binding fragment of the disclosure comprises a modified Avelumab (Bavencio, 451238, KXG2PJ551I, MSB-0010682, MSB-0010718C, PF-06834635, CAS 1537032-82-8: EMD Serono, Merck & Co., Merck KGaA, Merck Serono, National Cancer Institute (NCI), Pfizer), Durvalumab (Imfinzi, 28×28×90 KV (UNII code), MEDI-4736, CAS 1428935-60-7: AstraZeneca, Celgene, Children's Hospital Los Angeles (CHLA), City of Hope National Medical Center, MedImmune, Memorial Sloan-Kettering Cancer Center, Mirati Therapeutics, National Cancer Institute (NCI), Samsung Medical Center (SMC), Washington University), Atezolizumab (Tecentriq, 52CMIOWC3Y, MPDL-3280A, RG-7446, RO-5541267, CAS 1380723-44-3: Academisch Medisch Centrum (AMC), Chugai Pharmaceutical, EORTC, Genentech, Immune Design (Merck & Co.), Memorial Sloan-Kettering Cancer Center, National Cancer Institute (NCI), Roche, Roche Center for Medical Genomics), Sugemalimab (CS-1001, WBP-3155: CStone Pharmaceuticals, EQRx, Pfizer), KN-046 (CAS 2256084 Mar. 2: Jiangsu Alphamab Biopharmaceuticals, Sinovent), APL-502 (CBT-502, TQB-2450: Apollomics, Jiangsu Chia Tai Tianqing Pharmaceutical), Envafolimab (3D-025, ASC-22, KN-035, hu56V1-Fc-m1, CAS 2102192-68-5: 3D Medicines, Ascletis, Jiangsu Alphamab Biopharmaceuticals, Suzhou Alphamab, Tracon Pharmaceuticals, Inc.), Bintrafusp alfa (M-7824, MSB-0011359C, NW9K8C1JN3, CAS 1918149 Jan. 5: EMD Serono, GlaxoSmithKline, Merck KGaA, National Cancer Institute (NCI)), STI-1014 (STI-A1014, ZKAB-001: Lee's Pharmaceutical, Sorrento Therapeutics), PD-L1 t-haNK (ImmunityBio, NantKwest), A-167 (HBM-9167, KL-A167: Harbour BioMed, Sichuan Kelun-Biotech Biopharmaceutical), IMC-001 (STI-3031, STI-A-1015, STI-A1015, ImmuneOncia Therapeutics, Sorrento Therapeutics), HTI-1088 (SHR-1316: Atridia, Jiangsu Hengrui), IO-103 (IO Biotech), CX-072 (CytomX Therapeutics), AUPM-170 (CA-170: Aurigene, Curis), GS-4224 (Gilead), ND-021 (NM21-1480, PRO-1480: CStone Pharmaceuticals, Numab Therapeutics), BNT-311 (DuoBody-PD-L1x4-1BB, GEN-1046: BioNTech, Genmab), BGB-A333 (BeiGene), IBI-322 (Innovent Biologics), NM-01 (Nanomab Technology, Shanghai First People's Hospital), LY-3434172 (Eli Lilly), LDP (Dragonboat Biopharmaceutical), CDX-527 (Celldex Therapeutics), IBI-318 (Innovent Biologics, Lilly), 89Zr-DFO-REGN3504 (Regeneron), ALPN-202 (CD80 vIgD-Fc: Alpine Immune Sciences), INCB-086550 (Incyte), LY-3415244 (Eli Lilly), SHR-1701 (Jiangsu Hengrui), JS-003 (JS003-30, JS003-SD: Shanghai Junshi Biosciences), HLX-20 (PL2 #3: Henlix Biotech, Shanghai Henlius Biotech), ES-101 (INBRX-105, INBRX-105-1: Elpiscience BioPharma, Inhibrx), MSB-2311 (MabSpace Biosciences), FS-118, FS118 mAb2, LAG-3/PD-L1 mAb2: F-star Therapeutics, Merck & Co., Merck KGaA), FAZ-053 (LAE-005: Laekna Therapeutics, Novartis), Lodapolimab (LY-3300054, NR4MAD6PPB, CAS 2118349-31-6: Eli Lilly), MCLA-145 (Incyte, Merus), BMS-189 (BMS-986189, PD-L1-Milla from Bristol-Myers Squibb), Cosibelimab (CK-301, TG-1501, CAS 2216751-26-5: Checkpoint Therapeutics, Dana-Farber Cancer Institute, Samsung Biologics, TG Therapeutics), IL-15Ralpha-SD/IL-15 (KD-033: Kadmon), WP-1066 (CAS 857064-38-1: M. D. Anderson Cancer Center, Moleculin Biotech), BMS-936559 (MDX-1105: Bristol-Myers Squibb, Medarex, National Institute Allergy Infect Dis.), BMS-986192 (Bristol-Myers Squibb), RC-98 (RemeGen), CD-200AR-L (CD200AR-L: OX2 Therapeutics, University of Minnesota), ATA-3271 (Atara Biotherapeutics), IBC-Ab002 (ImmunoBrain Checkpoint), BMX-101 (Biomunex Pharmaceuticals), AVA-04-VbP (Avacta), ACE-1708 (Acepodia Biotech), KY-1043 (Kymab, Provenance Biopharmaceuticals), ACE-05 (YBL-013: Y-Biologics), ONC-0055 (ONC0055, PRS-344 S-095012: Pieris Pharmaceuticals, Servier), TLJ-1-CK (I-Mab Biopharma), GR-1405 (Chinese Academy of Medical Sciences), PD-1ACR-T (Taipei Medical University), N-809 (N-IL15/PD-L1: ImmunityBio), CB-201 (Crescendo Biologics), MEDI-1109 (MedImmune), AVA-004 (AVA-04: Avacta), CA-327 (Aurigene, Curis), ALN-PDL (Alnylam Pharmaceuticals), KY-1003 (Kymab), CD22 (aPD-L1) CAR-T cells (SL-22P: Hebei Senlang Biotechnology), ATA-2271 (M28z1XXPD-1DNR CAR T cells: Atara Biotherapeutics), and Zeushield cytotoxic T lymphocytes (Second Xiangya Hosp Central South Univ.), or a modified version of any of these, or a VH and VL of any of these, or the CDRs of any of these.

In some embodiments, the anti-PD-L1 antibody is Avelumab, Durvalumab, Atezolizumab, Sugemalimab, Envafolimab, Lodapolimab, or Cosibelimab, or a modified version thereof, or a VH and VL of any of these, or the CDRs of any of these. In some embodiments, the anti-PD-L1 antibody is Avelumab, Durvalumab, Atezolizumab, Sugemalimab, Envafolimab, Lodapolimab, or Cosibelima or a VH and VL of any of these, or the CDRs of any of these. In some embodiments, the antibody is a biosimilar of Avelumab, Durvalumab, Atezolizumab, Sugemalimab, Envafolimab, Lodapolimab, or Cosibelimab, or a VH and VL of any of these, or the CDRs of any of these.

TABLE 1 provides the sequences of exemplary anti-PD-L1 antibodies and anti-PD-L1 antigen binding fragments, and VHs and VLs thereof, that can be prepared in fusion immunocytokines as described herein. TABLE 1 also provides exemplary combinations of CDRs that can be utilized in a modified anti-PD-L1 fusion immunocytokine. Reference to an anti-PD-L1 antibody herein may alternatively refer to an anti-PD-L1 antigen binding fragment.

In some embodiments, an anti-PD-L1 antibody or an anti-PD-L1 antigen binding fragment comprises a heavy chain or VH having an amino acid sequence of any one of SEQ ID NOS: 232, 234, 236, 238, 242, 244, or 248, or a portion corresponding to a VH thereof. An anti-PD-L1 antibody or an anti-PD-L1 antigen binding fragment comprises a light chain or VL having an amino acid sequence of any one of SEQ ID NOS: 233, 235, 237, 239, 243, 245, or 249, or a portion corresponding to a VL thereof. In one instance, an anti-PD-L1 antibody or an anti-PD-L1 antigen binding fragment comprises a heavy chain or VH having an amino acid sequence shown in SEQ ID NO: 232, and a light chain or VL having an amino acid sequence shown in SEQ ID NO: 233. In another instance, an anti-PD-L1 antibody or an anti-PD-L1 antigen binding fragment comprises a heavy chain or VH having an amino acid sequence shown in SEQ ID NO: 234, and a light chain or VL having an amino acid sequence shown in SEQ ID NO: 235. In another instance, an anti-PD-L1 antibody or an anti-PD-L1 antigen binding fragment comprises a heavy chain or VH having an amino acid sequence shown in SEQ ID NO: 236, and a light chain or VL having an amino acid sequence shown in SEQ ID NO: 237. In another instance, an anti-PD-L1 antibody or an anti-PD-L1 antigen binding fragment comprises a heavy chain or VH having an amino acid sequence shown in SEQ ID NO: 238, and a light chain or VL having an amino acid sequence shown in SEQ ID NO: 239. In another instance, an anti-PD-L1 antibody or an anti-PD-L1 antigen binding fragment comprises a VH having an amino acid sequence shown in SEQ ID NO: 242, and a VL having an amino acid sequence shown in SEQ ID NO: 243. In another instance, an anti-PD-L1 antibody or an anti-PD-L1 antigen binding fragment comprises a VH having an amino acid sequence shown in SEQ ID NO: 244, and a VL having an amino acid sequence shown in SEQ ID NO: 245. In another instance, an anti-PD-L1 antibody or an anti-PD-L1 antigen binding fragment comprises a heavy chain or VH having an amino acid sequence shown in SEQ ID NO: 248, and a light chain or VL having an amino acid sequence shown in SEQ ID NO: 249.

In one instance, an anti-PD-L1 antibody or an anti-PD-L1 antigen binding fragment comprises a VH CDR1 having an amino acid sequence of SEQ ID NO: 250, a VH CDR2 having an amino acid sequence of SEQ ID NO: 251, a VH CDR3 having an amino acid sequence of SEQ ID NO: 252, VL CDR1 having an amino acid sequence of SEQ ID NO: 253, a VL CDR2 having an amino acid sequence of SEQ ID NO: 254, and a VL CDR3 having an amino acid sequence of SEQ ID NO: 255.

In one instance, an anti-PD-L1 antibody comprises a single domain binding antibody having an amino acid sequence of SEQ ID NO: 256, a tri-specific fusion single chain antibody construct having an amino acid sequence of SEQ ID NO: 257, or a bispecific tetrameric antibody like engager having an amino acid sequence of SEQ ID NO: 258.

TABLE 1
Exemplary Antibodies for Immune Cell Associated Antigens

| Antibody | | | |
|---|---|---|---|
| or Ag- | | | SEQ |
| binding | Antigen | | ID |
| fragment | Bound | Sequence | NO |
| Tislelizumab, | PD-1 | QVQLQESGPGLVKPSETLSLTCTVSGFSLTSYGVHWIRQPPGK | 332 |
| Baizean, | | GLEWIGVIYADGSTNYNPSLKSRVTISKDTSKNQVSLKLSSVT | |
| OKVO411B3N, | | AADTAVYYCARAYGNYWYIDVWGQGTTVTVSSASTKGPSVFPL | |
| BGB- | | APCSRSTSESTAALGCLVKDYFPEPVTVSWNSGALTSGVHTFPA | |
| A317, | | VLQSSGLYSLSSVVTVPSSSLGTKTYTCNVDHKPSNTKVDKRVES | |
| hu317-1/ | | KYGPPCPPCPAPPVAGGPSVFLFPPKPKDTLMISRTPEVTCVVVA | |
| IgG4mt2 | | VSQEDPEVQFNWYVDGVEVHNAKTKPREEQFNSTYRVVSVLTVV | |
| Heavy | | HQDWLNGKEYKCKVSNKGLPSSIEKTISKAKGQPREPQVYTLPPS | |
| Chain (VH | | QEEMTKNQVSLTCLVKGFYPSDIAVEWESNGQPENNYKTTPPVL | |
| in Bold) | | DSDGSFFLYSKLTVDKSRWQEGNVFSCSVMHEALHNHYTQKSLS | |
| | | LSLGK | |
| Tislelizumab, | PD-1 | DIVMTQSPDSLAVSLGERATINCKSSESVSNDVAWYQQKPGQP | 333 |
| Baizean, | | PKLLINYAFHRFTGVPDRFSGSGYGTDFTLTISSLQAEDVAVY | |
| OKVO411B3N, | | YCHQAYSSPYTFGQGTKLEIKRTVAAPSVFIFPPSDEQLKSGT | |
| BGB- | | ASVVCLLNNFYPREAKVQWKVDNALQSGNSQESVTEQDSKDST | |
| A317, | | YSLSSTLTLSKADYEKHKVYACEVTHQGLSSPVTKSFNRGEC | |
| hu317-1/ | | | |
| IgG4mt2 | | | |
| Light Chain | | | |
| (VL in | | | |
| Bold) | | | |
| Sintilimab, | PD-1 | QVQLVQSGAEVKKPGSSVKVSCKASGGTFSSYAISWVRQAPG | 334 |
| Tyvyt, IBI- | | QGLEWMGLIIPMEDTAGYAQKFQGRVAITVDESTSTAYMELS | |
| 308 Heavy | | SLRSEDTAVYYCARAEHSSTGTFDYWGQGTLVTVSSASTKGPS | |
| Chain (VH | | VFPLAPCSRSTSESTAALGCLVKDYFPEPVTVSWNSGALTSGVHT | |
| in Bold) | | FPAVLQSSGLYSLSSVVTVPSSSLGTKTYTCNVDHKPSNTKVDKR | |
| | | VESKYGPPCPPCPAPEFLGGPSVFLFPPKPKDTLMISRTPEVTCV | |
| | | VVDVSQEDPEVQFNWYVDGVEVHNAKTKPREEQFNSTYRVVSVL | |
| | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLGK | |
| Sintilimab, | PD-1 | DIQMTQSPSSVSASVGDRVTITCRASQGISSWLAWYQQKPGKA | 335 |
| Tyvyt, IBI- | | PKLLISAASSLQSGVPSRFSGSGSGTDFTLTISSLQPEDFATYY | |
| 308 Light | | CQQANHLPFTFGGGTKVEIKRTVAAPSVFIFPPSDEQLKSGTAS | |
| Chain (VL | | VVCLLNNFYPREAKVQWKVDNALQSGNSQESVTEQDSKDSTYS | |
| in Bold) | | LSSTLTLSKADYEKHKVYACEVTHQGLSSPVTKSFNRGEC | |
| Toripalimab, | PD-1 | QGQLVQSGAEVKKPGASVKVSCKASGYTFTDYEMHWVRQA | 336 |
| TeRuiPuLi, | | PIHGLEWIGVIESETGGTAYNQKFKGRVTITADKSTSTAYMEL | |
| Terepril, | | SSLRSEDTAVYYCAREGITTVATTYYWYFDVWGQGTTVTVSSA | |
| Tuoyi, JS- | | STKGPSVFPLAPCSRSTSESTAALGCLVKDYFPEPVTVSWNSGA | |
| 001, TAB- | | LTSGVHTFPAVLQSSGLYSLSSVVTVPSSSLGTKTYTCNVDHKP | |
| 001 Heavy | | SNTKVDKRVESKYGPPCPPCPAPEFLGGPSVFLFPPKPKDTLMI | |
| Chain (VH | | SRTPEVTCVVVDVSQEDPEVQFNWYVDGVEVHNAKTKPREEQFN | |
| in Bold) | | STYRVVSVLTVLHQDWLNGKEYKCKVSNKGLPSSIEKTISKAKG | |
| | | QPREPQVYTLPPSQEEMTKNQVSLTCLVKGFYPSDIAVEWESNG | |
| | | QPENNYKTTPPVLDSDGSFFLYSRLTVDKSRWQEGNVFSCSVMH | |
| | | EALHNHYTQKSLSLSLGK | |
| Toripalimab, | PD-1 | DVVMTQSPLSLPVTLGQPASISCRSSQSIVHSNGNTYLEWYLQ | 337 |
| TeRuiPuLi, | | KPGQSPQLLIYKVSNRFSGVPDRESGSGSGTDFTLKISRVEAED | |
| Terepril, | | VGVYYCFQGSHVPLTFGQGTKLEIKRTVAAPSVFIFPPSDEQLK | |
| Tuoyi, JS- | | SGTASVVCLLNNFYPREAKVQWKVDNALQSGNSQESVTEQDSK | |
| 001, TAB- | | DSTYSLSSTLTLSKADYEKHKVYACEVTHQGLSSPVTKSFNRGEC | |
| 001 Light | | | |
| Chain (VL | | | |
| in Bold) | | | |
| Camrelizumab, | PD-1 | EVQLVESGGGLVQPGGSLRLSCAASGFTFSSYMMSWVRQAP | 338 |
| HR- | | GKGLEWVATISGGGANTYYPDSVKGRFTISRDNAKNSLYLQM | |
| 301210, | | NSLRAEDTAVYYCARQLYYFDYWGQGTTVTVSSASTKGPSVF | |
| INCSHR- | | PLAPCSRSTSESTAALGCLVKDYFPEPVTVSWNSGALTSGVHTFP | |
| 01210, | | AVLQSSGLYSLSSVVTVPSSSLGTKTYTCNVDHKPSNTKVDKRVE | |
| SHR-1210 | | SKYGPPCPPCPAPEFLGGPSVFLFPPKPKDTLMISRTPEVTCVVVD | |
| Heavy | | VSQEDPEVQFNWYVDGVEVHNAKTKPREEQFNSTYRVVSVLTV | |
| Chain (VH | | LHQDWLNGKEYKCKVSNKGLPSSIEKTISKAKGQPREPQVYTLPP | |
| in Bold) | | SQEEMTKNQVSLTCLVKGFYPSDIAVEWESNGQPENNYKTTPPV | |
| | | LDSDGSFFLYSRLTVDKSRWQEGNVFSCSVMHEALHNHYTQKSL | |
| | | SLSLGK | |
| Camrelizumab, | PD-1 | DIQMTQSPSSLSASVGDRVTITCLASQTIGTWLTWYQQKPGK | 339 |
| HR- | | APKLLIYTATSLADGVPSRESGSGSGTDFTLTISSLQPEDFATY | |
| 301210, | | YCQQVYSIPWTFGGGTKVEIKRTVAAPSVFIFPPSDEQLKSGTA | |
| INCSHR- | | SVVCLLNNFYPREAKVQWKVDNALQSGNSQESVTEQDSKDSTYS | |
| 01210, | | LSSTLTLSKADYEKHKVYACEVTHQGLSSPVTKSFNRGEC | |
| SHR-1210 | | | |
| Light Chain | | | |
| (VL in | | | |
| Bold) | | | |
| Cemiplimab, | PD-1 | EVQLLESGGVLVQPGGSLRLSCAASGFTFSNFGMTWVRQAPG | 340 |
| Cemiplimab | | KGLEWVSGISGGGRDTYFADSVKGRFTISRDNSKNTLYLQMN | |
| -rwlc, | | SLKGEDTAVYYCVKWGNIYFDYWGQGTLVTVSSASTKGPSVF | |
| LIBTAYO®, | | PLAPCSRSTSESTAALGCLVKDYFPEPVTVSWNSGALTSGVHTFP | |
| 6QVL057INT, | | AVLQSSGLYSLSSVVTVPSSSLGTKTYTCNVDHKPSNTKVDKRVE | |
| H4H7798N, | | SKYGPPCPPCPAPEFLGGPSVFLFPPKPKDTLMISRTPEVTCVVVD | |
| REGN- | | VSQEDPEVQFNWYVDGVEVHNAKTKPREEQFNSTYRVVSVLTV | |
| 2810, SAR- | | LHQDWLNGKEYKCKVSNKGLPSSIEKTISKAKGQPREPQVYTLPP | |
| 439684 | | SQEEMTKNQVSLTCLVKGFYPSDIAVEWESNGQPENNYKTTPPV | |
| Heavy | | LDSDGSFFLYSRLTVDKSRWQEGNVFSCSVMHEALHNHYTQKSL | |
| Chain (VH | | SLSLGK | |
| in Bold) | | | |
| Cemiplimab, | PD-1 | DIQMTQSPSSLSASVGDSITITCRASLSINTFLNWYQQKPGKAP | 341 |
| Cemiplimab | | NLLIYAASSLHGGVPSRESGSGSGTDFTLTIRTLQPEDFATYYC | |
| -rwlc, | | QQSSNTPFTFGPGTVVDFRRTVAAPSVFIFPPSDEQLKSGTASVV | |
| LIBTAYO®, | | CLLNNFYPREAKVQWKVDNALQSGNSQESVTEQDSKDSTYSLSS | |
| 6QVL057INT, | | TLTLSKADYEKHKVYACEVTHQGLSSPVTKSFNRGEC | |
| H4H7798N, | | | |
| REGN- | | | |
| 2810, SAR- | | | |
| 439684 | | | |
| Light Chain | | | |
| (VL in | | | |
| Bold) | | | |
| Lambrolizumab, | PD-1 | QVQLVQSGVEVKKPGASVKVSCKASGYTFTNYYMYWVRQAP | 346 |
| Pembrolizumab, | | GQGLEWMGGINPSNGGTNFNEKFKNRVTLTTDSSTTTAYME | |
| KEYTRUDA®, | | LKSLQFDDTAVYYCARRDYRFDMGFDYWGQGTTVTVSSAST | |
| MK-3475, | | KGPSVFPLAPCSRSTSESTAALGCLVKDYFPEPVTVSWNSGALTS | |
| SCH- | | GVHTFPAVLQSSGLYSLSSVVTVPSSSLGTKTYTCNVDHKPSNTK | |
| 900475, | | VDKRVESKYGPPCPPCPAPEFLGGPSVFLFPPKPKDTLMISRTPEV | |
| h409A11 | | TCVVVDVSQEDPEVQFNWYVDGVEVHNAKTKPREEQFNSTYRV | |
| Heavy | | VSVLTVLHQDWLNGKEYKCKVSNKGLPSSIEKTISKAKGQPREPQ | |
| Chain (VH | | VYTLPPSQEEMTKNQVSLTCLVKGFYPSDIAVEWESNGQPENNY | |
| in Bold) | | KTTPPVLDSDGSFFLYSRLTVDKSRWQEGNVFSCSVMHEALHNH | |
| | | YTQKSLSLSLGK | |
| Lambrolizumab, | PD-1 | EIVLTQSPATLSLSPGERATLSCRASKGVSTSGYSYLHWYQQK | 347 |
| Pembrolizumab, | | PGQAPRLLIYLASYLESGVPARFSGSGSGTDFTLTISSLEPEDF | |
| KEYTRUDA®, | | AVYYCQHSRDLPLTFGGGTKVEIKRTVAAPSVFIFPPSDEQLKS | |
| MK-3475, | | GTASVVCLLNNFYPREAKVQWKVDNALQSGNSQESVTEQDSKD | |
| SCH- | | STYSLSSTLTLSKADYEKHKVYACEVTHQGLSSPVTKSFNRGEC | |
| 900475, | | | |
| h409A11 | | | |
| Light | | | |
| Chain (VL | | | |
| in Bold) | | | |
| Of | PD-1 | QVQLVQSGVEVKKPGASVKVSCKASGYTFTNYYMYWVRQAPG | 348 |
| Lambrolizumab, | | QGLEWMGGFPSNGGTNFNEKFKNRVTLTTDSSTTTAYMELKSLQ | |
| Pembrolizumab, | | FDDTAVYYCARRDYRFDMGFDYWGQGTTVTVSS | |
| KEYTRUDA®, | | | |
| MK-3475, | | | |
| SCH- | | | |
| 900475, | | | |
| h409A11 | | | |
| VH | | | |
| Lambrolizumab, | PD-1 | EIVLTQSPATLSLSPGERATLSCRASKGVSTSGYSYLHWYQQKPG | 349 |
| Pembrolizumab, | | QAPRLLIYLASYLESGVPARFSGSGSGTDFTLTISSLEPEDFAVYYC | |
| KEYTRUDA®, | | QHSRDLPLTFGGGTKVEIK | |
| MK-3475, | | | |
| SCH-900475, | | | |
| h409A11 | | | |
| VL | | | |
| Nivolumab, | PD-1 | QVQLVESGGGVVQPGRSLRLDCKASGITFSNSGMHWVRQAPGK | 350 |
| Nivolumab | | GLEWVAVIWYDGSKRYYADSVKGRFTISRDNSKNTLFLQMNSLR | |
| BMS, | | AEDTAVYYCATNDDYWGQGTLVTVSS | |
| OPDIVO®, | | | |
| BMS- | | | |
| 936558, | | | |
| MDX-1106, | | | |
| ONO-4538 | | | |
| VH | | | |
| Nivolumab, | PD-1 | EIVLTQSPATLSLSPGERATLSCRASQSVSSYLAWYQQKPGQAPR | 351 |
| Nivolumab | | LLIYDASNRATGIPARFSGSGSGTDFTLTISSLEPEDFAVYYCQQSS | |
| BMS, | | NWPRTFGQGTKVEIK | |
| OPDIVO®, | | | |
| BMS- | | | |
| 936558, | | | |
| MDX-1106, | | | |
| ONO-4538 | | | |
| VL | | | |
| Prolgolimab, | PD-1 | QVQLVQSGGGLVQPGGSLRLSCAASGFTFSSYWMYWVRQVP | 352 |
| Forteca, | | GKGLEWVSAIDTGGGRTYYADSVKGRFAISRVNAKNTMYLQ | |
| BCD-100 | | MNSLRAEDTAVYYCARDEGGGTGWGVLKDWPYGLDAWGQGT | |
| Heavy | | LVTVSSASTKGPSVFPLAPSSKSTSGGTAALGCLVKDYFPEPV | |
| Chain (VH | | TVSWNSGALTSGVHTFPAVLQSSGLYSLSSVVTVPSSSLGTQTYIC | |
| in Bold) | | NVNHKPSNTKVDKRVEPKSCDKTHTCPPCPAPEAAGGPSVFLFPP | |
| | | KPKDTLMISRTPEVTCVVVDVSHEDPEVKFNWYVDGVEVHNAK | |
| | | TKPREEQYNSTYRVVSVLTVLHQDWLNGKEYKCKVSNKALPAPI | |
| | | EKTISKAKGQPREPQVYTLPPSREEMTKNQVSLTCLVKGFYPSDI | |
| | | AVEWESNGQPENNYKTTPPVLDSDGSFFLYSKLTVDKSRWQQGN | |
| | | VFSCSVMHEALHNHYTQKSLSLSPGK | |
| Prolgolimab, | PD-1 | QPVLTQPLSVSVALGQTARITCGGNNIGSKNVHWYQQKPGQ | 353 |
| Forteca, | | APVLVIYRDSNRPSGIPERFSGSNSGNTATLTISRAQAGDEADY | |
| BCD-100 | | YCQVWDSSTAVFGTGTKLTVLQRTVAAPSVFIFPPSDEQLKSGT | |
| Light Chain | | ASVVCLLNNFYPREAKVQWKVDNALQSGNSQESVTEQDSKDST | |
| (VL in | | YSLSSTLTLSKADYEKHKVYACEVTHQGLSSPVTKSFNRGEC | |
| Bold) | | | |
| Balstilimab, | PD-1 | QVQLVESGGGVVQPGRSLRLSCAASGFTFSSYGMHWVRQAP | 354 |
| 1Q2QT5M7 | | GKGLEWVAVIWYDGSNKYYADSVKGRFTISRDNSKNTLYLQ | |
| EO, AGEN- | | MNSLRAEDTAVYYCASNGDHWGQGTLVTVSSASTKGPSVFPL | |
| 2034, | | APCSRSTSESTAALGCLVKDYFPEPVTVSWNSGALTSGVHTFPAV | |
| AGEN- | | LQSSGLYSLSSVVTVPSSSLGTKTYTCNVDHKPSNTKVDKRVESK | |
| 2034w | | YGPPCPPCPAPEFLGGPSVFLFPPKPKDTLMISRTPEVTCVVVDVS | |
| Heavy | | QEDPEVQFNWYVDGVEVHNAKTKPREEQFNSTYRVVSVLTVLH | |
| Chain (VH | | QDWLNGKEYKCKVSNKGLPSSIEKTISKAKGQPREPQVYTLPPSQ | |
| in Bold) | | EEMTKNQVSLTCLVKGFYPSDIAVEWESNGQPENNYKTTPPVLD | |
| | | SDGSFFLYSRLTVDKSRWQEGNVFSCSVMHEALHNHYTQKSLSL | |
| | | SLG | |
| Balstilimab, | PD-1 | EIVMTQSPATLSVSPGERATLSCRASQSVSSNLAWYQQKPGQ | 355 |
| 1Q2QT5M7 | | APRLLIYGASTRATGIPARFSGSGSGTEFTLTISSLQSEDFAVYY | |
| EO, AGEN- | | CQQYNNWPRTFGQGTKVEIKRTVAAPSVFIFPPSDEQLKSGTAS | |
| 2034, | | VVCLLNNFYPREAKVQWKVDNALQSGNSQESVTEQDSKDSTYS | |
| AGEN- | | LSSTLTLSKADYEKHKVYACEVTHQGLSSPVTKSFNRGEC | |
| 2034w | | | |
| Light Chain | | | |
| (VL in | | | |
| Bold) | | | |
| Dostarlimab, | PD-1 | EVQLLESGGGLVQPGGSLRLSCAASGFTFSSYDMSWVRQAPG | 356 |
| ANB-011, | | KGLEWVSTISGGGSYTYYQDSVKGRFTISRDNSKNTLYLQMN | |
| GSK- | | SLRAEDTAVYYCASPYYAMDYWGQGTTVTVSSASTKGPSVFP | |
| 4057190A, | | LAPCSRSTSESTAALGCLVKDYFPEPVTVSWNSGALTSGVHTFPA | |
| POGVQ9A4 | | VLQSSGLYSLSSVVTVPSSSLGTKTYTCNVDHKPSNTKVDKRVES | |
| S5, TSR- | | KYGPPCPPCPAPEFLGGPSVFLFPPKPKDTLMISRTPEVTCVVVDV | |
| 042, WBP- | | SQEDPEVQFNWYVDGVEVHNAKTKPREEQFNSTYRVVSVLTVL | |
| 285 Heavy | | HQDWLNGKEYKCKVSNKGLPSSIEKTISKAKGQPREPQVYTLPPS | |
| Chain (VH | | QEEMTKNQVSLTCLVKGFYPSDIAVEWESNGQPENNYKTTPPVL | |
| in Bold) | | DSDGSFFLYSRLTVDKSRWQEGNVFSCSVMHEALHNHYTQKSLS | |
| | | LSLGK | |
| Dostarlimab, | PD-1 | DIQLTQSPSFLSAYVGDRVTITCKASQDVGTAVAWYQQKPGK | 357 |
| ANB-011, | | APKLLIYWASTLHTGVPSRFSGSGSGTEFTLTISSLQPEDFATY | |
| GSK- | | YCQHYSSYPWTFGQGTKLEIKRTVAAPSVFIFPPSDEQLKSGTA | |
| 4057190A, | | SVVCLLNNFYPREAKVQWKVDNALQSGNSQESVTEQDSKDSTYS | |
| POGVQ9A4 | | LSSTLTLSKADYEKHKVYACEVTHQGLSSPVTKSFNRGEC | |
| S5, TSR- | | | |
| 042, WBP- | | | |
| 285 Light | | | |
| Chain (VL | | | |
| in Bold) | | | |
| Serplulimab, | PD-1 | QVQLVESGGGLVKPGGSLRLSCAASGFTFSNYGMSWIRQAPG | 358 |
| HLX-10 | | KGLEWSTISGGGSNIYYADSVKGRFTISRDNAKNSLYLQMNSL | |
| Heavy | | RAEDTAVYYCVSYYYGIDFWGQGTSVTVSSASKYGPSVFPLAPC | |
| Chain (VH | | SRSTSESTAALGCLVKDYFPEPVTVSWNSGALTSGVHTFPAVLQS | |
| in Bold) | | SGLYSLSSVVTVPSSSLGTKTYTCNVDHKPSNTKVDKRVESKYGP | |
| | | PCPPCPAPEFLGGPSVFLFPPKPKDTLMISRTPEVTCVVVDVSQED | |
| | | PEVQFNWYVDGVEVHNAKTKPREEQFNSTYRVVSVVLTVLHQD | |
| | | WLNGKEYKCKVSNKGLPSSIEKTISKAKGQPREPQVYTLPPSQEE | |
| | | MTKNQVSLTCLVKGFYPSDIAVEWESNGQPENNYKTTPPVLDSD | |
| | | GSFFLYSRLTVDKSRWQEGNVFSCSVMHEALHNHYTQKSLSLSL | |
| | | GK | |
| Serplulimab, | PD-1 | DIQMTQSPSSLSASVGDRVTITCKASQDVTTAVAWYQQKPGK | 359 |
| HLX-10 | | APKLLIYWASTRHTGVPSRFSGSGSGTDFTLTISSLQPEDFATY | |
| Light Chain | | YCQQHYTIPWTFGGGTKLEIKRTVAAPSVFIFPPSDEQLKSGTA | |
| (VL in | | SVVCLLNNFYPREAKVQWKVDNALQSGNSQESVTEQDSKDSTYS | |
| Bold) | | LSSTLTLSKADYEKHKVYACEVTHQGLSSPVTKSFNRGEC | |
| Retifanlimab, | PD-1 | QVQLVQSGAEVKKPGASVKVSCKASGYSFTSYWMNWVRQAP | 360 |
| 2Y3T5IF01Z, | | GQGLEWIGVIHPSDSETWLDQKFKDRVTITVDKSTSTAYMEL | |
| INCMGA- | | SSLRSEDTAVYYCAREHYGTSPFAYWGQGTLVTVSSASTKGPS | |
| 00012, | | VFPLAPCSRSTSESTAALGCLVKDYFPEPVTVSWNSGALTSGVHT | |
| INCMGA- | | FPAVLQSSGLYSLSSVVTVPSSSLGTKTYTCNVDHKPSNTKVDKR | |
| 0012, MGA- | | VESKYGPPCPPCPAPEFLGGPSVFLFPPKPKDTLMISRTPEVTCVV | |
| 012 Heavy | | VDVSQEDPEVQFNWYVDGVEVHNAKTKPREEQFNSTYRVVSVL | |
| Chain (VH | | TVLHQDWLNGKEYKCKVSNKGLPSSIEKTISKAKGQPREPQVYT | |
| in Bold) | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Retifanlimab, | PD-1 | EIVLTQSPATLSLSPGERATLSCRASESVDNYGMSFMNWFQQ | 361 |
| 2Y3T5IF01Z, | | KPGQPPKLLIHAASNQGSGVPSRFSGSGSGTDFTLTISSLEPED | |
| INCMGA- | | FAVYFCQQSKEVPYTFGGGTKVEIKRTVAAPSVFIFPPSDEQLK | |
| 00012, | | SGTASVVCLLNNFYPREAKVQWKVDNALQSGNSQESVTEQDSK | |
| INCMGA- | | DSTYSLSSTLTLSKADYEKHKVYACEVTHQGLSSPVTKSFNRGEC | |
| 0012, MGA- | | | |
| 012 Light | | | |
| Chain (VL | | | |
| in Bold) | | | |
| Sasanlimab, | PD-1 | QVQLVQSGAEVKKPGASVKVSCKASGYTFTSYWINWVRQAP | 362 |
| LZZOIC2E | | GQGLEWMGNIYPGSSLTNYNEKFKNRVTMTRDTSTSTVYME | |
| WP, PF- | | LSSLRSEDTAVYYCARLSTGTFAYWGQGTLVTVSSASTKGPSV | |
| 06801591, | | FPLAPCSRSTSESTAALGCLVKDYFPEPVTVSWNSGALTSGVHTF | |
| RN-888 | | PAVLQSSGLYSLSSVVTVPSSSLGTKTYTCNVDHKPSNTKVDKRV | |
| Heavy | | ESKYGPPCPPCPAPEFLGGPSVFLFPPKPKDTLMISRTPEVTCVVV | |
| Chain (VH | | DVSQEDPEVQFNWYVDGVEVHNAKTKPREEQFNSTYRVVSVLT | |
| in Bold) | | VLHQDWLNGKEYKCKVSNKGLPSSIEKTISKAKGQPREPQVYTLP | |
| | | PSQEEMTKNQVSLTCLVKGFYPSDIAVEWESNGQPENNYKTTPP | |
| | | VLDSDGSFFLYSRLTVDKSRWQEGNVFSCSVMHEALHNHYTQKS | |
| | | LSLSLGK | |
| Sasanlimab, | PD-1 | DIVMTQSPDSLAVSLGERATINCKSSQSLWDSGNQKNFLTWY | 363 |
| LZZOIC2E | | QQKPGQPPKLLIYWTSYRESGVPDRESGSGSGTDFTLTISSLQ | |
| WP, PF- | | AEDVAVYYCQNDYFYPHTFGGGTKVEIKRTVAAPSVFIFPPSDE | |
| 06801591, | | QLKSGTASVVCLLNNFYPREAKVQWKVDNALQSGNSQESVTEQ | |
| RN-888 | | DSKDSTYSLSSTLTLSKADYEKHKVYACEVTHQGLSSPVTKSFNR | |
| Light | | GEC | |
| Chain (VL | | | |
| in Bold) | | | |
| Spartalizumab, | PD-1 | EVQLVQSGAEVKKPGESLRISCKGSGYTFTTYWMHWVRQAT | 364 |
| NVP- | | GQGLEWMGNIYPGTGGSNFDEKFKNRVTITADKSTSTAYME | |
| LZV-184, | | LSSLRSEDTAVYYCTRWTTGTGAYWGQGTTVTVSSASTKGPS | |
| PDR-001, | | VFPLAPCSRSTSESTAALGCLVKDYFPEPVTVSWNSGALTSGVHT | |
| QOG25L6Z | | FPAVLQSSGLYSLSSVVTVPSSSLGTKTYTCNVDHKPSNTKVDKR | |
| 8Z Heavy | | VESKYGPPCPPCPAPEFLGGPSVFLFPPKPKDTLMISRTPEVTCVV | |
| Chain (VH | | VDVSQEDPEVQFNWYVDGVEVHNAKTKPREEQFNSTYRVVSVL | |
| in Bold) | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Spartalizumab, | PD-1 | EIVLTQSPATLSLSPGERATLSCKSSQSLLDSGNQKNFLTWYQ | 365 |
| NVP- | | QKPGQAPRLLIYWASTRESGVPSRFSGSGSGTDFTFTISSLEAE | |
| LZV-184, | | DAATYYCQNDYSYPYTFGQGTKVEIKRTVAAPSVFIFPPSDEQL | |
| PDR-001, | | KSGTASVVCLLNNFYPREAKVQWKVDNALQSGNSQESVTEQDS | |
| QOG25L6Z | | KDSTYSLSSTLTLSKADYEKHKVYACEVTHQGLSSPVTKSFNRGE | |
| 8Z Light | | C | |
| Chain (VL | | | |
| in Bold) | | | |
| Cetrelimab, | PD-1 | QVQLVQSGAEVKKPGSSVKVSCKASGGTFSSYAISWVRQAPG | 366 |
| JNJ-3283, | | QGLEWMGGIIPIFDTANYAQKFQGRVTITADESTSTAYMELSS | |
| JNJ- | | LRSEDTAVYYCARPGLAAAYDTGSLDYWGQGTLVTVSSASTK | |
| 63723283, | | GPSVFPLAPCSRSTSESTAALGCLVKDYFPEPVTVSWNSGALTSG | |
| LYK98WP9 | | VHTFPAVLQSSGLYSLSSVVTVPSSSLGTKTYTCNVDHKPSNTKV | |
| 1F Heavy | | DKRVESKYGPPCPPCPAPEFLGGPSVFLFPPKPKDTLMISRTPEVT | |
| Chain (VH | | CVVVDVSQEDPEVQFNWYVDGVEVHNAKTKPREEQFNSTYRVV | |
| in Bold) | | SVLTVLHQDWLNGKEYKCKVSNKGLPSSIEKTISKAKGQPREPQV | |
| | | YTLPPSQEEMTKNQVSLTCLVKGFYPSDIAVEWESNGQPENNYK | |
| | | TTPPVLDSDGSFFLYSRLTVDKSRWQEGNVFSCSVMHEALHNHY | |
| | | TQKSLSLSLGK | |
| Cetrelimab, | PD-1 | EIVLTQSPATLSLSPGERATLSCRASQSVRSYLAWYQQKPGQA | 367 |
| JNJ-3283, | | PRLLIYDASNRATGIPARFSGSGSGTDFTLTISSLEPEDFAVYYC | |
| JNJ- | | QQRNYWPLTFGQGTKVEIKRTVAAPSVFIFPPSDEQLKSGTASV | |
| 63723283, | | VCLLNNFYPREAKVQWKVDNALQSGNSQESVTEQDSKDSTYSLS | |
| LYK98WP9 | | STLTLSKADYEKHKVYACEVTHQGLSSPVTKSFNRGEC | |
| 1F Light | | | |
| Chain (VL | | | |
| in Bold) | | | |
| Tebotelimab, | PD-1 | DIQMTQSPSSLSASVGDRVTITCRASQDVSSVVAWYQQKPGK | 368 |
| MGD-013 | | APKLLIYSASYRYTGVPSRFSGSGSGTDFTLTISSLQPEDFATY | |
| Heavy | | YCQQHYSTPWTFGGGTKLEIKGGGSGGGGQVQLVQSGAEVKK | |
| Chain (VL | | PGASVKVSCKASGYSFTSYWMNWVRQAPGQGLEWIGVIHPSDSE | |
| in Bold) | | TWLDQKFKDRVTITVDKSTSTAYMELSSLRSEDTAVYYCAREHY | |
| | | GTSPFAYWGQGTLVTVSSGGCGGGEVAACEKEVAALEKEVAAL | |
| | | EKEVAALEKESKYGPPCPPCPAPEFLGGPSVFLFPPKPKDTLYITR | |
| | | EPEVTCVVVDVSQEDPEVQFNWYVDGVEVHNAKTKPREEQFNS | |
| | | TYRVVSVLTVLHQDWLNGKEYKCKVSNKGLPSSIEKTISKAKGQ | |
| | | PREPQVYTLPPSQEEMTKNQVSLTCLVKGFYPSDIAVEWESNGQP | |
| | | ENNYKTTPPVLDSDGSFFLYSRLTVDKSRWQEGNVFSCSVMHEA | |
| | | LHNHYTQKSLSLSLG | |
| Tebotelimab, | PD-1 | EIVLTQSPATLSLSPGERATLSCRASESVDNYGMSFMNWFQQ | 369 |
| MGD-013 | | KPGQPPKLLIHAASNQGSGVPSRFSGSGSGTDFTLTISSLEPED | |
| Light Chain | | FAVYFCQQSKEVPYTFGGGTKVEIKGGGSGGGGQVQLVQSGA | |
| (VL in | | EVKKPGASVKVSCKASGYTFTDYNMDWVRQAPGQGLEWMGDI | |
| Bold) | | NPDNGVTIYNQKFEGRVTMTTDTSTSTAYMELRSLRSDDTAVYY | |
| | | CAREADYFYFDYWGQGTTLTVSSGGCGGGKVAACKEKVAALKE | |
| | | KVAALKEKVAALKE | |
| Pidilizumab, | PD-1 | QVQLVQSGSELKKPGASVKISCKASGYTFTNYGMNWVRQAP | 370 |
| CT-011, | | GQGLQWMGWINTDSGESTYAEEFKGRFVFSLDTSVNTAYLQI | |
| MDV-9300 | | TSLTAEDTGMYFCVRVGYDALDYWGQGTLVTVSSASTKGPSV | |
| Heavy | | FPLAPSSKSTSGGTAALGCLVKDYFPEPVTVSWNSGALTSGVHTF | |
| Chain (VH | | PAVLQSSGLYSLSSVVTVPSSSLGTQTYICNVNHKPSNTKVDKRV | |
| in Bold) | | EPKSCDKTHTCPPCPAPELLGGPSVFLFPPKPKDTLMISRTPEVTC | |
| | | VVVDVSHEDPEVKFNWYVDGVEVHNAKTKPREEQYNSTYRVVS | |
| | | VLTVLHQDWLNGKEYKCKVSNKALPAPIEKTISKAKGQPREPQV | |
| | | YTLPPSREEMTKNQVSLTCLVKGFYPSDIAVEWESNGQPENNYK | |
| | | TTPPVLDSDGSFFLYSKLTVDKSRWQQGNVFSCSVMHEALHNHY | |
| | | TQKSLSLSPGK | |
| Pidilizumab, | PD-1 | EIVLTQSPSSLSASVGDRVTITCSARSSVSYMHWFQQKPGKAP | 371 |
| CT-011, | | KLWIYRTSNLASGVPSRESGSGSGTSYCLTINSLQPEDFATYYC | |
| MDV-9300 | | QQRSSFPLTFGGGTKLEIKRTVAAPSVFIFPPSDEQLKSGTASVV | |
| Light Chain | | CLLNNFYPREAKVQWKVDNALQSGNSQESVTEQDSKDSTYSLSS | |
| (VL in | | TLTLSKADYEKHKVYACEVTHQGLSSPVTKSFNRGEC | |
| Bold) | | | |
| SG-001 VH | PD-1 | QVQLVESGGGVVQPGRSLRLTCKASGLTFSSSGMHWVRQAPGK | 372 |
| | | GLEWVAVIWYDGSKRYYADSVKGRFTISRDNSKNTLFLQMNSLR | |
| | | AEDTAVYYCATNNDYWGQGTLVTVSS | |
| SG-001 VL | PD-1 | EIVLTQSPATLSLSPGERATLSCRASQSVSSYLAWYQQKPGQAPR | 373 |
| | | LLIYTASNRATGIPARFSGSGSGTDFTLTISSLEPEDFAVYYCQQYS | |
| | | NWPRTFGQGTKVEIK | |
| mpLZM- | PD-1 | EVQLQQSGPVLVKPGASVKMSCKASGYTFTSYYMYWVKQSHGK | 374 |
| 009 VH | | SLEWIGGVNPSNGGTNFNEKFKSKATLTVDKSSSTAYMELNSLTS | |
| (Murine | | EDSAVYYCARRDYRYDMGFDYWGQGTTLTVSS | |
| Precursor of | | | |
| LZM-009) | | | |
| mpLZM- | PD-1 | QIVLTQSPAIMSASPGEKVTMTCRASKGVSTSGYSYLHWYQQKP | 375 |
| 009 VL | | GSSPRLLIYLASYLESGVPVRFSGSGSGTSYSLTISRMEAEDAATY | |
| (Murine | | YCQHSRELPLTFGTGTRLEIK | |
| Precursor of | | | |
| LZM-009) | | | |
| LZM-009 | PD-1 | QVQLVQSGAEVKKPGASVKVSCKASGYTFTSYYMYWVRQAPG | 376 |
| VH | | QGLEWMGGVNPSNGGTNFNEKFKSRVTITADKSTSTAYMELSSL | |
| | | RSEDTAVYYCARRDYRYDMGFDYWGQGTTVTVSS | |
| LZM-009 | PD-1 | EIVLTQSPATLSLSPGERATISCRASKGVSTSGYSYLHWYQQKPG | 377 |
| VL | | QAPRLLIYLASYLESGVPARFSGSGSGTDFTLTISSLEPEDFATYYC | |
| | | QHSRELPLTFGTGTKVEIK | |
| Budigalimab, | PD-1 | EIQLVQSGAEVKKPGSSVKVSCKASGYTFTHYGMNWVRQAP | 378 |
| 6VDO4TY3OO, | | GQGLEWVGWVNTYTGEPTYADDFKGRLTFTLDTSTSTAYME | |
| ABBV- | | LSSLRSEDTAVYYCTREGEGLGFGDWGQGTTVTVSSASTKGP | |
| 181, PR- | | SVFPLAPSSKSTSGGTAALGCLVKDYFPEPVTVSWNSGALTSGVH | |
| 1648817 | | TFPAVLQSSGLYSLSSVVTVPSSSLGTQTYICNVNHKPSNTKVDK | |
| Heavy | | KVEPKSCDKTHTCPPCPAPEAAGGPSVFLFPPKPKDTLMISRTPEV | |
| Chain (VH | | TCVVVDVSHEDPEVKFNWYVDGVEVHNAKTKPREEQYNSTYRV | |
| in Bold) | | VSVLTVLHQDWLNGKEYKCKVSNKALPAPIEKTISKAKGQPREP | |
| | | QVYTLPPSREEMTKNQVSLTCLVKGFYPSDIAVEWESNGQPENN | |
| | | YKTTPPVLDSDGSFFLYSKLTVDKSRWQQGNVFSCSVMHEALHN | |
| | | HYTQKSLSLSPGK | |
| Budigalimab, | PD-1 | DVVMTQSPLSLPVTPGEPASISCRSSQSIVHSHGDTYLEWYLQ | 379 |
| 6VDO4TY3OO, | | KPGQSPQLLIYKVSNRFSGVPDRFSGSGSGTDFTLKISRVEAED | |
| ABBV- | | VGVYYCFQGSHIPVTFGQGTKLEIKRTVAAPSVFIFPPSDEQLKS | |
| 181, PR- | | GTASVVCLLNNFYPREAKVQWKVDNALQSGNSQESVTEQDSKD | |
| 1648817 | | STYSLSSTLTLSKADYEKHKVYACEVTHQGLSSPVTKSFNRGEC | |
| Light Chain | | | |
| (VL in | | | |
| Bold) | | | |
| Lambrolizumab, | PD-1 | NYYMY | 380 |
| Pembrolizumab, | | | |
| KEYTRUDA®, | | | |
| MK- | | | |
| 3475, SCH- | | | |
| 900475, | | | |
| h409A11 | | | |
| VH CDR1 | | | |
| Lambrolizumab, | PD-1 | GINPSNGGTNFNEKFKN | 381 |
| Pembrolizumab, | | | |
| KEYTRUDA®, | | | |
| MK- | | | |
| 3475, SCH- | | | |
| 900475, | | | |
| h409A11 | | | |
| VH CDR2 | | | |
| Lambrolizumab, | PD-1 | RDYRFDMGFDY | 382 |
| Pembrolizumab, | | | |
| KEYTRUDA®, | | | |
| MK- | | | |
| 3475, SCH- | | | |
| 900475, | | | |
| h409A11 | | | |
| VH CDR3 | | | |
| Lambrolizumab, | PD-1 | RASKGVSTSGYSYLH | 383 |
| Pembrolizumab, | | | |
| KEYTRUDA®, | | | |
| MK- | | | |
| 3475, SCH- | | | |
| 900475, | | | |
| h409A11 | | | |
| VL CDR1 | | | |
| Lambrolizumab, | PD-1 | LASYLES | 384 |
| Pembrolizumab, | | | |
| KEYTRUDA®, | | | |
| MK- | | | |
| 3475, SCH- | | | |
| 900475, | | | |
| h409A11 | | | |
| VL CDR2 | | | |
| Lambrolizumab, | PD-1 | QHSRDLPLT | 385 |
| Pembrolizumab, | | | |
| KEYTRUDA®, | | | |
| MK- | | | |
| 3475, SCH- | | | |
| 900475, | | | |
| h409A11 | | | |
| VL CDR3 | | | |
| Nivolumab, | PD-1 | NSGMH | 386 |
| Nivolumab | | | |
| BMS, | | | |
| OPDIVO®, | | | |
| BMS- | | | |
| 936558, | | | |
| MDX-1106, | | | |
| ONO-4538 | | | |
| VH CDR1 | | | |
| Nivolumab, | PD-1 | VIWYDGSKRYYADSVKG | 387 |
| Nivolumab | | | |
| BMS, | | | |
| OPDIVO®, | | | |
| BMS- | | | |
| 936558, | | | |
| MDX-1106, | | | |
| ONO-4538 | | | |
| VH CDR2 | | | |
| Nivolumab, | PD-1 | NDDY | 388 |
| Nivolumab | | | |
| BMS, | | | |
| OPDIVO®, | | | |
| BMS- | | | |
| 936558, | | | |
| MDX-1106, | | | |
| ONO-4538 | | | |
| VH CDR3 | | | |
| Nivolumab, | PD-1 | RASQSVSSYLA | 389 |
| Nivolumab | | | |
| BMS, | | | |
| OPDIVO®, | | | |
| BMS- | | | |
| 936558, | | | |
| MDX-1106, | | | |
| ONO-4538 | | | |
| VL CDR1 | | | |
| Nivolumab, | PD-1 | DASNRAT | 390 |
| Nivolumab | | | |
| BMS, | | | |
| OPDIVO®, | | | |
| BMS- | | | |
| 936558, | | | |
| MDX-1106, | | | |
| ONO-4538 | | | |
| VL CDR2 | | | |
| Nivolumab, | PD-1 | QQSSNWPRT | 391 |
| Nivolumab | | | |
| BMS, | | | |
| OPDIVO®, | | | |
| BMS- | | | |
| 936558, | | | |
| MDX-1106, | | | |
| ONO-4538 | | | |
| VL CDR3 | | | |
| Serplulimab, | PD-1 | FTFSNYGMS | 392 |
| HLX-10 | | | |
| VH CDR1 | | | |
| Serplulimab, | PD-1 | TISGGGSNIY | 393 |
| HLX-10 | | | |
| VH CDR2 | | | |
| Serplulimab, | PD-1 | VSYYYGIDF | 394 |
| HLX-10 | | | |
| VH CDR3 | | | |
| Serplulimab, | PD-1 | KASQDVTTAVA | 395 |
| HLX-10 | | | |
| VL CDR1 | | | |
| Serplulimab, | PD-1 | WASTRHT | 396 |
| HLX-10 | | | |
| VL CDR2 | | | |
| Serplulimab, | PD-1 | QQHYTIPWT | 397 |
| HLX-10 | | | |
| VL CDR3 | | | |
| SG-001 VH | PD-1 | GLTFSSSG | 398 |
| CDR1 | | | |
| SG-001 VH | PD-1 | IWYDGSKR | 399 |
| CDR2 | | | |
| SG-001 VH | PD-1 | ATNNDY | 400 |
| CDR3 | | | |
| SG-001 VL | PD-1 | RASQSVSSYLA | 401 |
| CDR1 | | | |
| SG-001 VL | PD-1 | TASNRAT | 402 |
| CDR2 | | | |
| SG-001 VL | PD-1 | QQYSNWPRT | 403 |
| CDR3 | | | |
| LZM-009 | PD-1 | GYTFTSYYMY | 404 |
| VH CDR1 | | | |
| LZM-009 | PD-1 | GVNPSNGGTNFNEKFKS | 405 |
| VH CDR2 | | | |
| LZM-009 | PD-1 | RDYRYDMGFDY | 406 |
| VH CDR3 | | | |
| LZM-009 | PD-1 | RASKGVSTSGYSYLH | 407 |
| VL CDR1 | | | |
| LZM-009 | PD-1 | LASYLE | 408 |
| VL CDR2 | | | |
| LZM-009 | PD-1 | QHSRELPLT | 409 |
| VL CDR3 | | | |
| Avelumab | PD-L1 | EVQLLESGGGLVQPGGSLRLSCAASGFTFSSYIMMWVRQAPG | 232 |
| (Generic) | | KGLEWVSSIYPSGGITFYADTVKGRFTISRDNSKNTLYLQMNS | |
| Bavencio | | LRAEDTAVYYCARIKLGTVTTVDYWGQGTLVTVSSASTKGPSV | |
| (Brand) | | FPLAPSSKSTSGGTAALGCLVKDYFPEPVTVSWNSGALTSGVH | |
| 451238 | | TFPAVLQSSGLYSLSSVVTVPSSSLGTQTYICNVNHKPSNTKV | |
| KXG2PJ551 | | DKKVEPKSCDKTHTCPPCPAPELLGGPSVFLFPPKPKDTLMIS | |
| I | | RTPEVTCVVVDVSHEDPEVKFNWYVDGVEVHNAKTKPREEQYN | |
| MSB- | | STYRVVSVLTVLHQDWLNGKEYKCKVSNKALPAPIEKTISKAK | |
| 0010682 | | GQPREPQVYTLPPSRDELTKNQVSLTCLVKGFYPSDIAVEWES | |
| MSB- | | NGQPENNYKTTPPVLDSDGSFFLYSKLTVDKSRWQQGNVFSCS | |
| 0010718C | | VMHEALHNHYTQKSLSLSPGK | |
| PF- | | | |
| 06834635 | | | |
| Heavy | | | |
| Chain (VH | | | |
| in Bold) | | | |
| Avelumab | PD-L1 | QSALTQPASVSGSPGQSITISCTGTSSDVGGYNYVSWYQQHPG | 233 |
| (Generic) | | KAPKLMIYDVSNRPSGVSNRFSGSKSGNTASLTISGLQAEDEA | |
| Bavencio | | DYYCSSYTSSSTRVFGTGTKVTVLGQPKANPTVTLFPPSSEEL | |
| (Brand) | | QANKATLVCLISDFYPGAVTVAWKADGSPVKAGVETTKPSKQS | |
| 451238 | | NNKYAASSYLSLTPEQWKSHRSYSCQVTHEGSTVEKTVAPTEC | |
| KXG2PJ551 | | S | |
| I | | | |
| MSB- | | | |
| 0010682 | | | |
| MSB- | | | |
| 0010718C | | | |
| PF- | | | |
| 06834635 | | | |
| Light Chain | | | |
| (VL in | | | |
| Bold) | | | |
| Durvalumab | PD-L1 | EVQLVESGGGLVQPGGSLRLSCAASGFTFSRYWMSWVRQAP | 234 |
| (Generic) | | GKGLEWVANIKQDGSEKYYVDSVKGRFTISRDNAKNSLYLQ | |
| Imfinzi | | MNSLRAEDTAVYYCAREGGWFGELAFDYWGQGTLVTVSSAS | |
| (Brand) | | TKGPSVFPLAPSSKSTSGGTAALGCLVKDYFPEPVTVSWNSGALT | |
| 28X28X90 | | SGVHTFPAVLQSSGLYSLSSVVTVPSSSLGTQTYICNVNHKPSNTK | |
| KV (UNII | | VDKRVEPKSCDKTHTCPPCPAPEFEGGPSVFLFPPKPKDTLMISRT | |
| code) | | PEVTCVVVDVSHEDPEVKFNWYVDGVEVHNAKTKPREEQYNST | |
| MEDI-4736 | | YRVVSVLTVLHQDWLNGKEYKCKVSNKALPASIEKTISKAKGQP | |
| Heavy | | REPQVYTLPPSREEMTKNQVSLTCLVKGFYPSDIAVEWESNGQPE | |
| Chain (VH | | NNYKTTPPVLDSDGSFFLYSKLTVDKSRWQQGNVFSCSVMHEAL | |
| in Bold) | | HNHYTQKSLSLSPGK | |
| Durvalumab | PD-L1 | EIVLTQSPGTLSLSPGERATLSCRASQRVSSSYLAWYQQKPGQ | 235 |
| (Generic) | | APRLLIYDASSRATGIPDRESGSGSGTDFTLTISRLEPEDFAVYY | |
| Imfinzi | | CQQYGSLPWTFGQGTKVEIKRTVAAPSVFIFPPSDEQLKSGTAS | |
| (Brand) | | VVCLLNNFYPREAKVQWKVDNALQSGNSQESVTEQDSKDSTYS | |
| 28X28X90 | | LSSTLTLSKADYEKHKVYACEVTHQGLSSPVTKSFNRGEC | |
| KV (UNII | | | |
| code) | | | |
| MEDI-4736 | | | |
| Light Chain | | | |
| (VL in | | | |
| Bold) | | | |
| Atezolizumab | PD-L1 | EVQLVESGGGLVQPGGSLRLSCAASGFTFSDSWIHWVRQAPG | 236 |
| (Generic) | | KGLEWVAWISPYGGSTYYADSVKGRFTISADTSKNTAYLQMN | |
| Tecentriq | | SLRAEDTAVYYCARRHWPGGFDYWGQGTLVTVSSASTKGPS | |
| (Brand) | | VFPLAPSSKSTSGGTAALGCLVKDYFPEPVTVSWNSGALTSGVHT | |
| 52CMIOWC | | FPAVLQSSGLYSLSSVVTVPSSSLGTQTYICNVNHKPSNTKVDKK | |
| 3Y | | VEPKSCDKTHTCPPCPAPELLGGPSVFLFPPKPKDTLMISRTPEVT | |
| MPDL- | | CVVVDVSHEDPEVKFNWYVDGVEVHNAKTKPREEQYASTYRVV | |
| 3280A | | SVLTVLHQDWLNGKEYKCKVSNKALPAPIEKTISKAKGQPREPQ | |
| RG-7446 | | VYTLPPSREEMTKNQVSLTCLVKGFYPSDIAVEWESNGQPENNY | |
| RO- | | KTTPPVLDSDGSFFLYSKLTVDKSRWQQGNVFSCSVMHEALHNH | |
| 5541267 | | YTQKSLSLSPGK | |
| Heavy | | | |
| Chain (VH | | | |
| in Bold) | | | |
| Atezolizumab | PD-L1 | DIQMTQSPSSLSASVGDRVTITCRASQDVSTAVAWYQQKPGK | 237 |
| (Generic) | | APKLLIYSASFLYSGVPSRFSGSGSGTDFTLTISSLQPEDFATYY | |
| Tecentriq | | CQQYLYHPATFGQGTKVEIKRTVAAPSVFIFPPSDEQLKSGTAS | |
| (Brand) | | VVCLLNNFYPREAKVQWKVDNALQSGNSQESVTEQDSKDSTYS | |
| 52CMIOWC | | LSSTLTLSKADYEKHKVYACEVTHQGLSSPVTKSFNRGEC | |
| 3Y | | | |
| MPDL- | | | |
| 3280A | | | |
| RG-7446 | | | |
| RO- | | | |
| 5541267 | | | |
| Light Chain | | | |
| (VL in | | | |
| Bold) | | | |
| Sugemalimab | PD-L1 | EVQLLESGGGLVQPGGSLRLSCAASGFTFSSYAMSWVRQAPG | 238 |
| (Generic) | | KGLEWVSGISGSGGFTYYADSVKGRFTISRDNSKNTLYLQMN | |
| CS-1001 | | SLRAEDTAVYYCAKPPRGYNYGPFDYWGQGTLVTVSSASTKG | |
| WBP-3155 | | PSVFPLAPCSRSTSESTAALGCLVKDYFPEPVTVSWNSGALTSGV | |
| Heavy | | HTFPAVLQSSGLYSLSSVVTVPSSSLGTKTYTCNVDHKPSNTKVD | |
| Chain (VH | | KRVESKYGPPCPPCPAPEFLGGPSVFLFPPKPKDTLMISRTPEVTC | |
| in Bold) | | VVVDVSQEDPEVQFNWYVDGVEVHNAKTKPREEQFNSTYRVVS | |
| | | VLTVLHQDWLNGKEYKCKVSNKGLPSSIEKTISKAKGQPREPQV | |
| | | YTLPPSQEEMTKNQVSLTCLVKGFYPSDIAVEWESNGQPENNYK | |
| | | TTPPVLDSDGSFFLYSRLTVDKSRWQEGNVFSCSVMHEALHNHY | |
| | | TQKSLSLSLGK | |
| Sugemalima | PD-L1 | SYVLTQPPSVSVAPGQTARITCGGNNIGSKSVHWYQQKPGQA | 239 |
| b (Generic) | | PVLVVYDDSDRPSGIPERFSGSNSGNTATLTISRVEAGDEADYY | |
| CS-1001 | | CQVWDSSSDHVVFGGGTKLTVLGQPKAAPSVTLFPPSSEELQA | |
| WBP-3155 | | NKATLVCLISDFYPGAVTVAWKADSSPVKAGVETTTPSKQSNNK | |
| Light | | YAASSYLSLTPEQWKSHRSYSCQVTHEGSTVEKTVAPTECS | |
| Chain (VL | | | |
| in Bold) | | | |
| JS-003 | PD-L1 | QGQLQESGPSLVKPSQTLSLTCTVSGDSITRGYWNWIRKHPGKGL | 242 |
| JS003-30 | | EYIGYISYTGSTYSNLSLKSRVTISRDTSKNQYYLKLSSVTAADTA | |
| JS003-SD | | VYYCATSTGWLDPVDYWGQGTLVTVSS | |
| VH | | | |
| JS-003 | PD-L1 | DIVMTQSPDSLAVSLGERATINCKASQNVDTSVA WFQQKPGQPP | 243 |
| JS003-30 | | KALIYSASFRYSGVPDRESGSGSGTDFTLTISSLQAEDVAVYFCQQ | |
| | | YYGYPFTFGQGTKLEIK | |
| JS003-SD | | | |
| VL | | | |
| HLX-20 | PD-L1 | EVQLVQSGGGLVKPGGSLRLSCAASGFTFSSYTMNWVRQAPGK | 244 |
| PL2#3 VH | | GLEWVSSISSGSDYLYYADSVKGRFTISRDNAKNSLYLQMNSLRA | |
| | | EDTAVYYCARNELRWYPQAGAFDRWGQGTMVTVSS | |
| HLX-20 | PD-L1 | QSVVTQPPSMSAAPGQRVTISCSGSSSYIESSYVGWYQQLPGTAP | 245 |
| PL2#3 VL | | RLLIYDDDMRPSGIPDRFSGSKSGTSATLAITGLQTGDEADYYCEI | |
| | | WRSGLGGVFGGGTKLTVL | |
| Lodapolima | PD-L1 | QVQLVQSGAEVKKPGSSVKVSCKASGGTFSSYAISWVRQAPG | 248 |
| b (Generic) | | QGLEWMGGIIPIFGTANYAQKFQGRVTITADKSTSTAYMELSS | |
| LY-3300054 | | LRSEDTAVYYCARSPDYSPYYYYGMDVWGQGTTVTVSSASTKG | |
| NR4MAD6 | | PSVFPLAPSSKSTSGGTAALGCLVKDYFPEPVTVSWNSGALTSG | |
| PPB Heavy | | VHTFPAVLQSSGLYSLSSVVTVPSSSLGTQTYICNVNHKPSNTKV | |
| Chain (VH | | DKRVEPKSCDKTHTCPPCPAPEAEGAPSVFLFPPKPKDTLMISRT | |
| in Bold) | | PEVTCVVVDVSHEDPEVKFNWYVDGVEVHNAKTKPREEQYNSTY | |
| | | RVVSVLTVLHQDWLNGKEYKCKVSNKALPSSIEKTISKAKGQPR | |
| | | EPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALH | |
| | | NHYTQKSLSLSPGK | |
| Lodapolima | PD-L1 | QSVLTQPPSASGTPGQRVTISCSGSSSNIGSNTVNWYQQLPGT | 249 |
| b (Generic) | | APKLLIYGNSNRPSGVPDRESGSKSGTSASLAISGLQSEDEADY | |
| LY-3300054 | | YCQSYDSSLSGSVFGGGIKLTVLGQPKAAPSVTLFPPSSEELQAN | |
| NR4MAD6 | | KATLVCLISDFYPGAVTVAWKADSSPVKAGVETTTPSKQSNNKY | |
| PPB Light | | AASSYLSLTPEQWKSHRSYSCQVTHEGSTVEKTVAPAECS | |
| Chain (VL | | | |
| in Bold) | | | |
| HLX-20 | PD-L1 | SYTMN | 250 |
| PL2#3 VH | | | |
| CDR1 | | | |
| HLX-20 | PD-L1 | SISSGSDYLYYADSVKG | 251 |
| PL2#3 VH | | | |
| CDR2 | | | |
| HLX-20 | PD-L1 | NELRWYPQAGAFDR | 252 |
| PL2#3 VH | | | |
| CDR3 | | | |
| HLX-20 | PD-L1 | SGSSSYIESSYVG | 253 |
| PL2#3 VL | | | |
| CDR1 | | | |
| HLX-20 | PD-L1 | DDDMRPS | 254 |
| PL2#3 VL | | | |
| CDR2 | | | |
| HLX-20 | PD-L1 | EIWRSGLGGV | 255 |
| PL2#3 VL | | | |
| CDR3 | | | |
| Envafolimab | PD-L1 | QVQLVESGGGLVQPGGSLRLSCAASGKMSSRRCMAWFRQAP | 256 |
| (Generic) | | GKERERVAKLLTTSGSTYLADSVKGRFTISRDNSKNTVYLQM | |
| 3D-025 | | NSLRAEDTAVYYCAADSFEDPTCTLVTSSGAFQYWGQGTLVT | |
| ASC-22 | | VSSEPKSSDKTHTCPPCPAPELLGGPSVFLFPPKPKDTLMISRTPEV | |
| KN-035 | | TCVVVAVSHEDPEVKFNWYVDGVEVHNAKTKPREEQYNSTYRV | |
| hu56V1-Fc- | | VSVLTVLHQDWLNGKEYKCKVSNKALPAGIEKTISKAKGQPREP | |
| ml single- | | QVYTLPPSRDELTKNQVSLTCLVKGFYPSDIAVEWESNGQPENNY | |
| domain | | KTTPPVLDSDGSFFLYSKLTVDKSRWQQGNVFSCSVMHEALHNH | |
| antibody | | YTQKSLSLSPGK | |
| (VH in | | | |
| Bold) | | | |
| ND-021 | PD-L1 | DIQMTQSPASLSASVGDRVTITCQASQSIGTYLAWYQQKPGKPPK | 257 |
| NM21-1480 | | LLIYRAFILASGVPSRFSGSGSGTDFTLTISSLQPEDFATYYCQSNF | |
| PRO-1480 | | YSDSTTIGPNAFGTGTKVTVLGGGGGSEVQLVESGGGLVQPGGS | |
| Tri-specific | | LRLSCAASGFSFSANYYPCWVRQAPGKGLEWIGCIYGGSSDITYD | |
| fusion | | ANWTKGRFTISRDNSKNTVYLQMNSLRAEDTAVYYCARSAWYS | |
| single-chain | | GWGGDLWGQGTLVTVSSGGGGSGGGGSGGGGSGGGGSIQMTQ | |
| antibody | | SPSSLSASVGDRVTITCQASQSISNRLAWYQQKPGKAPKLLIYSAS | |
| construct | | TLASGVPSRFSGSGSGTDFTLTISSLQPEDFATYYCQSTYYGNDGN | |
| | | AFGTGTKVTVLGGGGGSEVQLVESGGGLVQPGGSLRLSCAASGF | |
| | | SFNSDYWIYWVRQAPGKGLEWIASIYGGSSGNTQYASWAQGRFT | |
| | | ISRDNSKNTVYLQMNSLRAEDTAVYFCARGYVDYGGATDLWGQ | |
| | | GTLVTVSSGGGGSGGGGSIQMTQSPSSLSASVGDRVTITCQSSESV | |
| | | YSNNQLSWYQQKPGQPPKLLIYDASDLASGVPSRFSGSGSGTDFT | |
| | | LTISSLQPEDFATYYCAGGFSSSSDTAFGGGTKLTVLGGGGGSGG | |
| | | GGSGGGGSGGGGSEVQLVESGGGLVQPGGSLRLSCAASGFSLSS | |
| | | NAMGWVRQAPGKGLEYIGIISVGGFTYYASWAKGRFTISRDNSK | |
| | | NTVYLQMNSLRAEDTATYFCARDRHGGDSSGAFYLWGQGTLVT | |
| | | VSS | |
| ACE-05 | PD-L1 | QMQLVQSGAEVKKPGSSVKVSCKASGGTFSSYAISWVRQAPGQG | 258 |
| YBL-013 | | LEWMGRIIPILGIANYAQKFQGRVTITADKSTSTAYMELSSLRSED | |
| Bispecific | | TAVYYCAKPRDGYNLVAFDIWGQGTMVTVSSASTKGPSVFPLAP | |
| tetrameric | | SSKSTSGGTAALGCLVKDYFPEPVTVSWNSGALTSGVHTFPAVL | |
| antibody- | | QSSGLYSLSSVVTVPSSSLGTQTYICNVNHKPSNTKVDKKVEPKS | |
| like cell | | CDKTHTCPPCPAPELLGGPGGGGSEVQLQQSGPELVKPGPSMKIS | |
| engager | | CKASGYSFTGYTMNWVKQSHGKNLEWMGLINPYKGVSTYNQK | |
| (ALICE) | | FKDKATLTVDKSSSTAYMELLSLTSEDSAVYYCARSGYYGDSDW | |
| comprising | | YFDVWGQGTTLTVFSQMQLVQSGAEVKKPGSSVKVSCKASGGT | |
| two identical | | FSSYAISWVRQAPGQGLEWMGRIIPILGIANYAQKFQGRVTITAD | |
| light chains | | KSTSTAYMELSSLRSEDTAVYYCAKPRDGYNLVAFDIWGQGTM | |
| (LC) | | VTVSSASTKGPSVFPLAPSSKSTSGGTAALGCLVKDYFPEPVTVS | |
| consisting of | | WNSGALTSGVHTFPAVLQSSGLYSLSSVVTVPSSSLGTQTYICNV | |
| antigen | | NHKPSNTKVDKKVEPKSCDKTHTCPPCPAPELLGGPGGGGSDIQ | |
| binding | | MTQTTSSLSASLGDRVTISCRASQDIRNYLNWYQQKPDGTVKLLI | |
| domains | | YYTSRLHSGVPSKFSGSGSGTDYSLTISNLEQEDIATYFCQQGNTL | |
| (ABDs) | | PWTFAGGTKLEIKRQLVLTQPPSVSGAPGQRVTISCTGSSSNIGAG | |
| targeting | | YDVHWYQQLPGAAPKLLIYGDINRPSGVPDRESGSKSGISASLAIT | |
| programmed | | GLQAEDEADYYCQSYDSSLSGGVFGGGTKLTVLRTVAAPSVFIFP | |
| cell death- | | PSDEQLKSGTASVVCLLNNFYPREAKVQWKVDNALQSGNSQESV | |
| ligand 1 | | TEQDSKDSTYSLSSTLTLSKADYEKHKVYACEVTHQGLSSPVTKS | |
| (PD-L1), | | FNRGEC | |
| and two | | | |
| different | | | |
| heavy chain | | | |
| (HC)-like | | | |
| chains | | | |
| (ACE-05- | | | |
| VH and | | | |
| ACE-05- | | | |
| VL) each | | | |
| consisting of | | | |
| an anti-PD- | | | |
| L1 ABD and | | | |
| an anti-CD3 | | | |
| ABD; | | | |
| wherein | | | |
| each HC | | | |
| comprises a | | | |
| G4S linker | | | |
| (SEQ ID | | | |
| NO: 410) | | | |
| between the | | | |
| hinge region | | | |
| and the | | | |
| second ABD | | | |

### Peptide Linkers

In some embodiments, the antibody or antigen binding fragment is fused to the IL-18 polypeptide through a peptide linker. Any fusion immunocytokine described herein can employ a peptide linker, unless otherwise specified (e.g., the IL-18 polypeptide is described as fused “directly” to the antibody or antigen binding fragment). In such instances, the linker comprises one or more peptide bonds between the antibody or antigen binding fragment and the IL-18 polypeptide. In some embodiments, the linker between the fusion protein of the antibody or antigen binding fragment and the IL-18 polypeptide is a linking peptide. Non-limiting examples of linking peptides include, but are not limited to (GS)ₙ (SEQ ID NO: 224), (GGS)ₙ (SEQ ID NO: 225), (GGGS)ₙ (SEQ ID NO: 226), (GGSG), (SEQ ID NO: 227), or (GGSGG)ₙ (SEQ ID NO: 228), (GGGGS)ₙ (SEQ ID NO: 229), wherein n is 1, 2, 3, 4, 5, 6, 7, 8, 9, or 10. For example, a linking peptide can be (GGGGS) 3 (SEQ ID NO: 230) or (GGGGS) 4 (SEQ ID NO: 231).

In some embodiments, the peptide linker can be or comprise a different structure, such as that of a hinge region derived from an antibody hinge region (e.g., that of an IgG1, IgG2, IgG3, or IgG4, or a derivative thereof). In some embodiments, the peptide linker comprises a hinge region derived from an antibody hinge region, or a portion thereof. In some embodiments, the peptide linker comprises a hnge region derived from an antibody hinge region, wherein the IL-18 polypeptide is fused to the N-terminus of an Fc domain.

### Domain Architecture and Constant Domain

A fusion immunocytokine as provided herein can be prepare according to a variety of different architectures/domain structures. A non-limiting exemplary set of such architectures are provide in FIGS. 2A-2F.

In some embodiments, the IL-18 polypeptide is fused via its N-terminus to the antibody or antigen binding fragment thereof. In some embodiments, the N-terminus of the IL-18 polypeptide is the first residue depicted in SEQ ID NO: 1. In some embodiments, the N-termunis of the IL-18 polypeptide is truncated relative to SEQ ID NO: 1 such that different position of SEQ ID NO: 1 acts is the N-terminus (e.g., residues at positions 2, 3, 4, or 5 of SEQ ID NO: 1).

In some embodiments, the IL-18 polypeptide is fused to a C-terminus of a light chain or a heavy chain, or a fragment thereof, of the antibody or antigen binding fragment thereof. In some embodiments, the IL-18 polypeptide is fused to the C-terminus of the light chain of the antibody or antigen binding fragment, or a portion thereof. In some embodiments, the IL-18 polypeptide is fused to the C-terrminus of a full length light chain of the antibody or antigen binding fragment thereof (e.g., to the C-terminus of the light chain constant region, such as a kappa or lambda light chain constant region), such as that in the format depicted in FIG. 2E.

In some embodiments, the IL-18 polypeptide is fused to the C-terminus of a heavy chain of the antibody or antigen binding fragment, or a portion thereof. In some embodiments, the IL-18 polypeptide is fused to the C-terrminus of a full length heavy chain of the antibody or antigen binding fragment thereof (e.g., to the C-terminus of the CH3 domain), such as that in the format depicted in FIG. 2A and FIG. 2C). In some embodiments, both arms of the fusion immunocytokine are fused to IL-18 polypeptides at the C-terminus of the heavy chain (e.g., as in FIG. 2C). In some embodiments, only a single arm of the fusion immunocytokine contains an IL-18 polypeptide attached to the C-terminus of the heavy chain (e.g., as in FIG. 2A). In such embodiments, the Fc domain can comprise modifications described herein for facilitation of assymetric pairings (i.e., heterodimerization) of the arms of the fusion immunocytokine (e.g., RF mutations, knob-into-hole modifications, etc.).

In some embodiments, the IL-18 polypeptide is fused via its C-terminus to the antibody or antigen binding fragment thereof. In some embodiments, the IL-18 polypeptide is fused to an N-terminus of a light chain or a heavy chain, or a fragment thereof, of the antibody or antigen binding fragment thereof. In some embodiments, the IL-18 polypeptide is fused to the N-terminus of a light chain of the antibody or antigen binding fragment thereof. In some embodiments, the IL-18 polypeptide is fused to the N-terminus of full-length light chain of the antibody or antigen binding fragment thereof. In some embodiments, the IL-18 polypeptide is fused to the N-terminus of a heavy chain of the antibody or antigen binding fragment thereof. In some embodiments, the IL-18 polypeptide is fused to the N-terminus of a full length heavy chain of the antibody or antigen binding fragment thereof (e.g., as in FIG. 2B or FIG. 2D). In some embodiments, both arms of the fusion immunocytokine are fused to IL-17 polypeptides at the N-terminus of the heavy chain (e.g., as in FIG. 2B). In some embodiments, only a single arm of the fusion immunocytokine contains an IL-18 polypeptide attached to the N-terminus of the heavy chain (e.g., as in FIG. 2D). In such embodiments, the Fc domain can comprise modification described herein for facilitation of asymmetric pairings (i.e., heterodimerization) of the arms of the fusion immunocytokine (e.g., RF mutations, knob-into-hole modifications, etc.).

In some embodiments, the fusion immunocytokine comprises only a single arm which has an antigen binding domain (e.g., a single VH domain and a single VL domain, or a single VH domain and no VL domain). An exemplary such fusion immunocytokine architecture is shown in FIG. 2F. In some embodiments, the fusion immunocytokine comprises a single full-length heavy chain and a second polypeptide comprising an Fc domain. In some embodiments, the Fc domain of the second polypeptide is fused to the IL-18 polypeptide (e.g., by either the N-or C-terminus of the Fc domain, optionally by a peptide linker).

In some embodiments, the fusion immunocytokine described herein comprises a) a first polypeptide comprising an antigen binding domain of the antibody or antigen binding fragment thereof. In some embodiments, the first polypeptide comprises an Fc domain. In some embodiments, the first polypeptide comprises a VH domain. In some embodiments, the first polypeptide comprises a full-length heavy chain. In some embodiments, the first polypeptide is bound to a light chain of the antibody or antigen binding fragment, or a portion thereof. In some embodiments, the first polypeptide is bound to a full-length light chain of the antibody or antigen binding fragment. In some embodiments, the first polypeptide comprises the IL-18 polypeptide. In some embodiments, the first polypeptide does not comprise the IL-18 polypeptide.

In some embodiments, the fusion immunocytokine comprises a second polypeptide. In some embodiments, the second polypeptide comprises an Fc domain. In some embodiments, the second polypeptide comprises the IL-18 polypeptide fused to the Fc domain. In some embodiments, the IL-18 polypeptide is fused to the C-terminus of the Fc domain. In some embodiments, the IL-18 polypeptide is fused to the N-terminus of the Fc domain (as in the fusion immunocytokine depicted in FIG. 2E). In some embodiments, the second polypeptide does not comprise an antigen binding domain of the antibody. In some embodiments, the Fc domain of the second polypeptide does not comprise the IL-18 polypeptide (e.g., the IL-18 polypeptide is fused to the first polypeptide which comprises an antigen binding domain, and the second polypeptide comprises no IL-18 polypeptide or antigen binding domain). In some embodiments, the second polypeptide also contains an antigen binding domain of the antibody or antigen binding fragment.

Exemplary fusion immunocytokine architectures are further provided in the table below. In the table below, the various domains are listed from the N-terminal to C-terminal direction. For reach attachment of the IL-18 polypeptide to the rest of the polypeptide in which it is comprised, it is contemplated that a peptide linker as described herein.

| Architecture | | | | |
|---|---|---|---|---|
| Structure | Arm 1 | | Arm 2 | |
| 1 | Light | Heavy Chain | Light | Heavy Chain |
| | Chain | | Chain | |
| 2 | VL-VC | VH-CH1-Hinge- | VL-VC | VH-CH1-Hinge- |
| | | Fc-IL-18 | | Fc-IL-18 |
| 3 | VL-VC | VH-CH1-Hinge- | VL-VC | VH-CH1-Hinge-Fc |
| | | Fc-IL-18 | | |
| 4 | VL-VC | IL-18-VH-CH1- | VL-VC | IL-18-VH-CH1- |
| | | Hinge-Fc | | Hinge-Fc |
| 5 | VL-VC | IL-18-VH-CH1- | VL-VC | VH-CH1-Hinge-Fc |
| | | Hinge-Fc | | |
| 6 | VL-VC- | VH-CH1-Hinge- | VL-VC- | VH-CH1-Hinge-Fc |
| | IL-18 | Fc | IL-18 | |
| 7 | IL-18- | VH-CH1-Hinge- | IL-18- | VH-CH1-Hinge-Fc |
| | VL-VC | Fc | VL-VC | |
| 8 | VL-VC | VH-CH1-Hinge- | N/A | IL-18-Fc |
| | | Fc | | |
| 9 | VL-VC | VH-CH1-Hinge- | N/A | Fc-IL-18 |
| | | Fc | | |
| 10 | VL-VC | CH-CH1-Hinge- | N/A | Fc |
| | | Fc-IL-18 | | |

In some embodiments, the fusion immunocytokine comprises two IL-18 polypeptides (i.e., two IL-18 polypeptide molecules fused to the antibody or antigen binding fragment thereof). In embodiments of such cases, the two IL-18 polypeptides are preferably identical, though it is not required. Similarly, in such cases where there are two IL-18 polypeptides, they are preferably configured such that they are comprised on identical arms of the fusion immunocytokine (e.g., as depicted in FIGS. 2B, 2C, and 2E), though this is not required. For example, a bi-specific fusion immunocytokine (i.e., a fusion immunocytokine which contains antigen binding domains which bind to two separate antigens) can comprise two IL-18 polypeptides affixed to arms which are different (e.g., fused to each non-identical heavy chain). In some embodiments, the two molecules of the IL-18 polypeptide are fused to different chains of the antibody or antigen-binding fragment thereof.

In some embodiments, the fusion immunocytokine comprises a single molecule of the IL-18 polypeptide. In such cases, the fusion immunocytokine can be a heterodimerized construct (e.g., contains non-identical Fc domains). In some embodiments, the antibody or antigen binding fragment thereof of the fusion immunocytokine comprises a first Fc domain and a second Fc domain. In some embodiments, the first and second Fc domains are not identical. In some embodiments, only one of the first and second Fc domains is comprised in a polypeptide chain which also comprises the IL-18 polypeptide.

In some embodiments, the first Fc domain and/or the second Fc domain comprise one or more modifications which favor heterodimerization of the first and second Fc domains. Many such modifications are known in art for generating bispecific antibodies which can be applied to the instant disclosure. Such modifications are described in, for example, “Fc Engineering for Developing Therapeutic Bispecific Antibodies and Novel Scaffolds,” Liu et al., Front. Immunol., 26 Jan. 2017 (doi.org/10.3389/fimmu.2017.00038) and include, for example, knob-into-hole technology (see, e.g., U.S. Pat. No. 8,216,805) and modification introduced into one Fc domain to abrogate binding to protein A to facilitate purification of desired heterodimeric formats (e.g., RF mutations, as described in, e.g., U.S. Pat. No. 11, 168,111). In some embodiments, the fusion immunocytokines provided herein utilize knob-into-hole technology, for example the “hole” modifications of Y349C, T366S, L368A, and Y407V and the “knob” modifications of S354C and T366W (EU numbering). In some embodiments, the fusion immunocytokines provided herein utilize the RF mutations, e.g., H435R and Y436F mutations. In some embodiments, the fusion immunocytokines utilize both of these modifications together (e.g., one arm of the fusion immunocytokine having the hole and RF modifications, and one arm of the fusion immunocytokine having the knob modifications). In some embodiments, on Fc domain comprises T336W, H435R, and Y436F substitutions and the other Fc domain comprises T366S, L368A, Y407V substitutions.

In some embodiments, the constant domains (e.g., the Fc domain) of a fusion immunocytokine described herein can comprise further modifications (either in place of or in addition to the other modifications described herein, such as those which favor heterodimerization of two different arms of the fusion immunocytokine). Such modifications to antibody Fc regions are known in the art and include, for example, modifications which alter antibody effector functions (e.g., enhance or decease Fc receptor binding or activity, thereby altering antibody-dependent cellular cytotoxicity, complement dependent cytotoxicity, or other effects), improve half-life circulation, or otherwise alter the performance of the molecule. Such modifications are well known in the art and are described in, for example, “Conceptual Approaches to Modulating Antibody Effector Functions and Circulation Half-Life,” Saunders et al., Fron. Immunol., 7 Jun. 2019 (doi.org/10.3389/fimmu.2019.01296). Such modifications can be at any relevant portion of the fusion immunocytokine, including without limitation an Fc domain (e.g., either the CH2 or CH3 domain, or both), a hinge region, a CH1 domain, a light chain constant region, and/or a framework region of an antigen binding domain (e.g., a VH or VL domain).

In some embodiments, the fusion immunocytokine comprises constant domains of or derived from (e.g., containing an artificial modification of) an IgG1, IgG2, IgG3, or IgG4. In some embodiments, the fusion immunocytokine comprises constant domains of or derived from an IgG4. In some embodiments, the fusion immunocytokine comprises light chain constant region of or derived from a kappa or lambda light chain.

In some embodiments, a constant domain comprises at least one constant domain having at least 80%, 85%, 90%, 91%, 92%, 93%, 94%, 95%, 96%, 97%, 98%, or 99% sequence identity to a sequence set forth in the table below. In some embodiments, each constant domain of the fusion immunocytokine has a sequence at least 80%, 85%, 90%, 91%, 92%, 93%, 94%, 95%, 96%, 97%, 98%, or 99% sequence identity to a sequence set forth in the table below.

| | SEQ ID | |
|---|---|---|
| Description | NO: | Sequence |
| CH1, | 101 | GPSVFPLAPCSRSTSESTAALGCLVKDYFPEPVTVSWN |
| Hinge A, | | SGALTSGVHTFPAVLQSSGLYSLSSVVTVPSSSLGTKT |
| CH2, and | | YTCNVDHKPSNTKVDKRVESKYGPPCPSCPAPEFLGG |
| CH3 | | PSVFLFPPKPKDTLMISRTPEVTCVVVDVSQEDPEVQF |
| domains | | NWYVDGVEVHNAKTKPREEQFNSTYRVVSVLTVLHQ |
| | | DWLNGKEYKCKVSNKGLPSSIEKTISKAKGQPREPQV |
| | | YTLPPSQEEMTKNQVSLTCLVKGFYPSDIAVEWESNG |
| | | QPENNYKTTPPVLDSDGSFFLYSRLTVDKSRWQEGNV |
| | | FSCSVMHEALHNHYTQKSLSLSLGK |
| CH1, | 102 | GPSVFPLAPCSRSTSESTAALGCLVKDYFPEPVTVSWN |
| Hinge B, | | SGALTSGVHTFPAVLQSSGLYSLSSVVTVPSSSLGTKT |
| CH2, and | | YTCNVDHKPSNTKVDKRVESKYGPPCPPCPAPEFLGG |
| CH3 | | PSVFLFPPKPKDTLMISRTPEVTCVVVDVSQEDPEVQF |
| domains | | NWYVDGVEVHNAKTKPREEQFNSTYRVVSVLTVLHQ |
| | | DWLNGKEYKCKVSNKGLPSSIEKTISKAKGQPREPQV |
| | | YTLPPSQEEMTKNQVSLTCLVKGFYPSDIAVEWESNG |
| | | QPENNYKTTPPVLDSDGSFFLYSRLTVDKSRWQEGNV |
| | | FSCSVMHEALHNHYTQKSLSLSLGK |
| CH2 | 103 | APEFLGGPSVFLFPPKPKDTLMISRTPEVTCVVVDVSQ |
| Domain | | EDPEVQFNWYVDGVEVHNAKTKPREEQFNSTYRVVS |
| | | VLTVLHQDWLNGKEYKCKVSNKGLPSSIEKTISKAK |
| CH3 | 104 | GQPREPQVYTLPPSQEEMTKNQVSLTCLVKGFYPSDI |
| Domain | | AVEWESNGQPENNYKTTPPVLDSDGSFFLYSRLTVDK |
| | | SRWQEGNVFSCSVMHEALHNHYTQKSLSLSLGK |
| Fc Domain | 105 | APEFLGGPSVFLFPPKPKDTLMISRTPEVTCVVVDVSQ |
| | | EDPEVQFNWYVDGVEVHNAKTKPREEQFNSTYRVVS |
| | | VLTVLHQDWLNGKEYKCKVSNKGLPSSIEKTISKAKG |
| | | QPREPQVYTLPPSQEEMTKNQVSLTCLVKGFYPSDIA |
| | | VEWESNGQPENNYKTTPPVLDSDGSFFLYSRLTVDKS |
| | | RWQEGNVFSCSVMHEALHNHYTQKSLSLSLGK |
| Fc Domain | 106 | APEFLGGPSVFLFPPKPKDTLMISRTPEVTCVVVDVSQ |
| with Y349C, | | EDPEVQFNWYVDGVEVHNAKTKPREEQFNSTYRVVS |
| T366S, L368A, | | VLTVLHQDWLNGKEYKCKVSNKGLPSSIEKTISKAKG |
| and Y407V | | QPREPQVCTLPPSQEEMTKNQVSLSCAVKGFYPSDIA |
| ″Hole″ | | VEWESNGQPENNYKTTPPVLDSDGSFFLVSRLTVDKS |
| mutations | | RWQEGNVFSCSVMHEALHNRFTQKSLSLSLGK |
| and H435R | | |
| and Y436F | | |
| ″RF″ | | |
| mutations | | |
| Fc domain | 107 | APEFLGGPSVFLFPPKPKDTLMISRTPEVTCVVVDVSQ |
| with T366S, | | EDPEVQFNWYVDGVEVHNAKTKPREEQFNSTYRVVS |
| L368A, | | VLTVLHQDWLNGKEYKCKVSNKGLPSSIEKTISKAKG |
| Y407V | | QPREPQVYTLPPSQEEMTKNQVSLSCAVKGFYPSDIA |
| substitutions | | VEWESNGQPENNYKTTPPVLDSDGSFFLVSRLTVDKS |
| | | RWQEGNVFSCSVMHEALHNHYTQKSLSLSLGK |
| Fc Domain | 108 | APEFLGGPSVFLFPPKPKDTLMISRTPEVTCVVVDVSQ |
| with | | EDPEVQFNWYVDGVEVHNAKTKPREEQFNSTYRVVS |
| S354C and | | VLTVLHQDWLNGKEYKCKVSNKGLPSSIEKTISKAKG |
| T366W | | QPREPQVYTLPPCQEEMTKNQVSLWCLVKGFYPSDIA |
| ″Knob″ | | VEWESNGQPENNYKTTPPVLDSDGSFFLYSRLTVDKS |
| mutations | | RWQEGNVFSCSVMHEALHNHYTQKSLSLSLGK |
| Fc Domain | 109 | APEFLGGPSVFLFPPKPKDTLMISRTPEVTCVVVDVSQ |
| with | | EDPEVQFNWYVDGVEVHNAKTKPREEQFNSTYRVVS |
| T366W, | | VLTVLHQDWLNGKEYKCKVSNKGLPSSIEKTISKAKG |
| H435R, | | QPREPQVYTLPPSQEEMTKNQVSLWCLVKGFYPSDIA |
| and Y436F | | VEWESNGQPENNYKTTPPVLDSDGSFFLYSRLTVDKS |
| substitutions | | RWQEGNVFSCSVMHEALHNRFTQKSLSLSLGK |
| Fc Domain | 110 | APEFLGGPSVFLFPPKPKDTLMISRTPEVTCVVVDVSQ |
| with | | EDPEVQFNWYVDGVEVHNAKTKPREEQFNSTYRVVS |
| T366W | | VLTVLHQDWLNGKEYKCKVSNKGLPSSIEKTISKAKG |
| substitution | | QPREPQVYTLPPSQEEMTKNQVSLWCLVKGFYPSDIA |
| | | VEWESNGQPENNYKTTPPVLDSDGSFFLYSRLTVDKS |
| | | RWQEGNVFSCSVMHEALHNHYTQKSLSLSLGK |
| Light | 111 | RTVAAPSVFIFPPSDEQLKSGTASVVCLLNNFYPREAK |
| Chain | | VQWKVDNALQSGNSQESVTEQDSKDSTYSLSSTLTLS |
| Constant | | KADYEKHKVYACEVTHQGLSSPVTKSFNRGEC |
| Domain | | |
| Hinge A | 112 | ESKYGPPCPSCP |
| Region | | |
| Hinge B | 113 | ESKYGPPCPPCP |
| Region | | |
| CH1 | 114 | GPSVFPLAPCSRSTSESTAALGCLVKDYFPEPVTVSWN |
| Domain | | SGALTSGVHTFPAVLQSSGLYSLSSVVTVPSSSLGTKT |
| | | YTCNVDHKPSNTKVDKRV |

### IL-18 Polypeptides

The present disclosure describes fusion immunocytokines comprising antibodies or antigen binding fragments fused to interleukin-18 (IL-18) polypeptides and their use as therapeutic agents. IL-18 is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a T helper type 1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Ra), which is the ligand binding chain for mature IL-18. However, the binding affinity of IL-18 to IL-18Ra is low. In cells that express the co-receptor, IL-18 receptor beta chain (IL-18RB), a high affinity heterodimer complex is formed, which then activates cell signaling.

The activity of IL-18 is balanced by the presence of a high affinity, naturally occurring IL-18 binding protein (IL-18BP). IL-18BP binds IL-18 and neutralizes the biological activity of IL-18. Cell surface IL-18Ra competes with IL-18BP for IL-18 binding. Increased disease severity can be associated with an imbalance of IL-18 to IL-18BP such that levels of free IL-18 are elevated in the circulation. FIG. 3 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Ra to inhibit IL-18 activity.

In some embodiments, the IL-18 polypeptides of the fusion immunocytokines provided herein display reduced binding to IL-18BP while retaining binding to the IL-18 receptor. In some embodiments, IL-18 polypeptides with this property provided herein are able to retain IL-18 receptor signaling activity (including inducing production of IFNy) even in the presence of IL-18BP. This allows the fusion immunocytokines provided herein to retain IL-18 signaling activity well beyond a short period of time after administration, or upon repeat administrations. In some embodiments, the IL-18 polypeptides with this property comprise a modification (e.g., substitution, polymer attachment, or deletion) at one or more amino acid residues which convey this property to the IL-18 polypeptide. Examples of IL-18 polypeptides with this property are provided herein and other variants are also known, such as those described in Patent Cooperation Treaty Publication No. WO2019051015A1, which is hereby incorporated by reference as if set forth herein in its entirety.

### Modifications to IL-18 Polypeptides

In some embodiments, the IL-18 polypeptide of the fusion immunocytokine comprises one or more modifications to that of SEQ ID NO: 1. The modifications provided herein are in addition to any modification at the point of attachment as discussed supra. In some embodiments, the residue position numbering of the IL-18 polypeptide is based on SEQ ID NO: 1 as a reference sequence.

Modifications to the IL-18 polypeptide described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the modification is in the range of amino acid residues 1-127, based on the sequence of human IL-1837-193 (SEQ ID NO: 1). SEQ ID NO: 1 reflects the bioactive form of IL-18. Endogenously, IL-18 is initially expressed with an additional 36 amino acid segment at the N-terminus which is cleaved by caspases to mediate biologic activity.

In some embodiments, the IL-18 polypeptide of the immunocytokine described herein contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues relative to SEQ ID NO: 1.

In some embodiments, the IL-18 polypeptide of the immunocytokine comprises an amino acid sequence having at least 80%, 85%, 90%, 91%, 92%, 93%, 94%, 95%, 96%, 97%, 98%, or 99% sequence identity to the sequence set forth in SEQ ID NO: 1.

In some embodiments, the IL-18 polypeptide of the fusion immunocytokine described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the IL-18 polypeptide comprises 1 to 9 amino acid substitutions. In some embodiments, the IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-18 polypeptide comprises at most 4 amino acid substitutions, 5 amino acid substitutions, 6 amino acid substitutions, 7 amino acid substitutions, or 9 amino acid substitutions. In some embodiments, the IL-18 polypeptide comprises up to 5, up to 6, up to 7, up to 8, up to 9, up to 10, or up to 15 amino acid substitutions relative to SEQ ID NO: 1.

Unless specifically mentioned otherwise, the residue position numbering of IL-18 polypepytides as provided in this disclosure is based on SEQ ID NO: 1 as a reference sequence.

In certain embodiments, the IL-18 polypeptide of the fusion immunocyotkine comprises a substitution at residue Y1. In certain embodiments, the IL-18 polypeptide can comprises Y1M substitution. Unless specifically mentioned otherwise, the amino acid substitutions provided in this paragraph, and elsewhere in this disclosure is with respect to SEQ ID NO: 1, as a reference sequence. In certain embodiments, the IL-18 polypeptide comprises a substitution at residue F2. In certain embodiments, the IL-18 polypeptide can comprises F2A substitution. In certain embodiments, the IL-18 polypeptide comprises a substitution at residue E6. In certain embodiments, the IL-18 polypeptide comprises E6K substitution. In certain embodiments, the IL-18 polypeptide comprises E6R substitution. In certain embodiments, the IL-18 polypeptide comprises a substitution at residue K8. In certain embodiments, the IL-18 polypeptide comprises K8L substitution. In certain embodiments, the IL-18 polypeptide comprises K8E substitution. In certain embodiments, the IL-18 polypeptide comprises K8R substitution. In certain embodiments, the IL-18 polypeptide comprises a substitution at residue V11. In certain embodiments, the IL-18 polypeptide can comprises V11I substitution. In certain embodiments, the IL-18 polypeptide comprises a substitution at residue E31. In certain embodiments, the IL-18 polypeptide comprises E31A substitution. In certain embodiments, the IL-18 polypeptide comprises a substitution at residue T34. In certain embodiments, the IL-18 polypeptide comprises T34A substitution. In certain embodiments, the IL-18 polypeptide comprises a substitution at residue D35. In certain embodiments, the IL-18 polypeptide comprises D35A substitution. In certain embodiments, the IL-18 polypeptide comprises a substitution at residue S36. In certain embodiments, the IL-18 polypeptide comprises S36A substitution. In certain embodiments, the IL-18 polypeptide comprises a substitution at residue D37. In certain embodiments, the IL-18 polypeptide comprises D37A substitution. In certain embodiments, the IL-18 polypeptide comprises a substitution at residue D40. In certain embodiments, the IL-18 polypeptide comprises D40A substitution. In certain embodiments, the IL-18 polypeptide comprises a substitution at residue N41. In certain embodiments, the IL-18 polypeptide comprises N41A substitution. In certain embodiments, the IL-18 polypeptide comprises a substitution at residue 149. In certain embodiments, the IL-18 polypeptide comprises I49E substitution. In certain embodiments, the IL-18 polypeptide comprises 149M substitution. In certain embodiments, the IL-18 polypeptide comprises I49R substitution. In certain embodiments, the IL-18 polypeptide comprises a substitution at residue K53. In certain embodiments, the IL-18 polypeptide comprises K53A substitution. In certain embodiments, the IL-18 polypeptide comprises a substitution at residue D54. In certain embodiments, the IL-18 polypeptide comprises D54A substitution. In certain embodiments, the IL-18 polypeptide comprises a substitution at residue S55. In certain embodiments, the IL-18 polypeptide comprises S55A substitution. In certain embodiments, the IL-18 polypeptide comprises S55T substitution. In certain embodiments, the IL-18 polypeptide comprises S55H substitution. In certain embodiments, the IL-18 polypeptide comprises S55R substitution. In certain embodiments, the IL-18 polypeptide comprises a substitution at residue T63. In certain embodiments, the IL-18 polypeptide comprises T63A substitution. In certain embodiments, the IL-18 polypeptide comprises a substitution at residue Q103. In certain embodiments, the IL-18 polypeptide comprises Q103R substitution. In certain embodiments, the IL-18 polypeptide comprises Q103E substitution. In certain embodiments, the IL-18 polypeptide comprises Q103K substitution. In certain embodiments, the IL-18 polypeptide comprises a substitution at residue G108. In certain embodiments, the IL-18 polypeptide comprises G108A substitution. In certain embodiments, the IL-18 polypeptide comprises a substitution at residue H109. In certain embodiments, the IL-18 polypeptide comprises H109A substitution. In certain embodiments, the IL-18 polypeptide comprises a substitution at residue D110. In certain embodiments, the IL-18 polypeptide comprises D110A substitution. In certain embodiments, the IL-18 polypeptide comprises a substitution at residue D132. In certain embodiments, the IL-18 polypeptide comprises D132A substitution. In certain embodiments, the IL-18 polypeptide comprises a substitution at residue V153. In certain embodiments, the IL-18 polypeptide comprises V153R substitution. In certain embodiments, the IL-18 polypeptide comprises V153E substitution. In certain embodiments, the IL-18 polypeptide comprises V153Y substitution. In certain embodiments, the IL-18 polypeptide comprises a substitution at residue C38. In certain embodiments, the IL-18 polypeptide comprises C38A substitution. In certain embodiments, the IL-18 polypeptide comprises C38S substitution. In certain embodiments, the IL-18 polypeptide comprises a substitution at residue C68. In certain embodiments, the IL-18 polypeptide comprises C68A substitution. In certain embodiments, the IL-18 polypeptide comprises C68S substitution. In certain embodiments, the IL-18 polypeptide comprises a substitution at residue C76. In certain embodiments, the IL-18 polypeptide comprises C76A substitution. In certain embodiments, the IL-18 polypeptide comprises C76S substitution. In certain embodiments, the IL-18 polypeptide comprises a substitution at residue C127. In certain embodiments, the IL-18 polypeptide comprises C127A substitution. In certain embodiments, the IL-18 polypeptide comprises C127S substitution. In certain embodiments, the IL-18 polypeptide comprises a substitution at residue C38, C68, C76, and/or C127. In certain embodiments, the IL-18 polypeptide comprises a C38A, C38S, C68A, C68S, C76A, C76S, C127A, and/or C127S substitution. In certain embodiments, the IL-18 polypeptide comprises C38A, C76A, and C127A substitutions. In certain embodiments, the IL-18 polypeptide comprises C38S, C76S and C127S substitutions.

In some embodiments, the IL-18 polypeptide of the fusion immunocytokine comprises at least one modification to the amino acid sequence of SEQ ID NO: 1 selected from: Y01X, F02X, E06X, S10X, V11X, D17X, C38X, M51X, K53X, D54X, S55X, T63X, C68X, C76X, AND C127X, wherein each X is independently a natural or non-natural amino acid. In some embodiments, the IL-18 polypeptide further comprises an amino acid substitution at the point of attachment of the linker, such as residue 69, residue 70, residue 85, residue 86, residue 95, or residue 98. In some embodiments, the IL-18 polypeptide comprises at least one modification to the amino acid sequence of SEQ ID NO: 1 selected from: Y01G, F02A, E06K, S10T, V11I, D17N, C38S, C38A, C38Q, M51G, K53A, D54A, S55A, T63A, C68S, C68A, C76S, C76A, C127A, and C127S. In some embodiments, the IL-18 polypeptide further comprises an amino acid substitution at the point of attachment of the linker, such as E69C, K70C, E85C, M86C, T95C, or D98C.

In certain embodiments, the IL-18 polypeptide comprises of the fusion immunocytokine i) a substitution at residue V11 and ii) at least one additional substitution at a residue selected from Y1, F2, E6, K8, S10, D17, T34, D35, S36, D37, D40, N41, 149, M51, K53, D54, S55, Q103, S105, G108, H109, D110, and D132.

In some embodiments, IL-18 polypeptide of the fusion immunocyotkine comprises E06K and K53A, wherein residue position numbering of the IL-18 polypeptide is based on SEQ ID NO: 1 as a reference sequence. In some embodiments, the IL-18 polypeptide further comprises V11I. In some embodiments, the IL-18 polypeptide further comprises T63A. In some embodiments, the IL-18 polypeptide further comprises at least one of Y01X, S55X, F02X, D54X, C38X, C68X, E69X, K70X, C76X, or C127X, wherein each X is independently an amino acid or an amino acid derivative. In some embodiments, the IL-18 polypeptide further comprises at least one of Y01G, S55A, F02A, D54A, C38S, C38A, C38Q, C68S, C68A, E69C, K70C, C76S, C76A, C127S, or C127A. In some embodiments, the IL-18 polypeptide further comprises an amino acid substitution at the point of attachment of the linker, such as residue 69, residue 70, residue 85, residue 86, residue 95, or residue 98.

In some embodiments, the IL-18 peptide the fusion immunocytokine comprises at least one modification to the amino acid sequence of SEQ ID NO: 1, wherein the modification is E06X, V11X, K53X, S55X, or T63X, wherein X is a natural or non-natural amino acid. In some embodiments, the IL-18 peptide comprises at least two modifications to the amino acid sequence of SEQ ID NO: 1, wherein the modifications comprise E06X and K53X; E06X and S55X; K53X and S55X; E06X and T63X; or K53X and T63X, wherein X is a natural or non-natural amino acid. In some embodiments, the IL-18 peptide comprises at least three modifications to the amino acid sequence of SEQ ID NO: 1, wherein the modifications comprise E06X, K53X, and S55X; or E06X, K53X, and T63X, wherein X is a natural or non-natural amino acid. In some embodiments, the IL-18 peptide comprises at least four modifications to the amino acid sequence of SEQ ID NO: 1, wherein the modifications comprise E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In each embodiment wherein a plurality of amino acids residues are replaced with a natural or non-natural amino acid X, each X is independently the same or a different amino acid.

In some embodiments, the IL-18 peptide of the fusion immunocytokine comprises at least one modification to the amino acid sequence of SEQ ID NO: 1, wherein the modification is E06K, V11I, K53A, S55A, or T63A. In some embodiments, the IL-18 peptide comprises at least two modifications to the amino acid sequence of SEQ ID NO: 1, wherein the modifications comprise E06K and K53A; E06K and S55A; K53A and S55A; E06K and T63A; or K53A and T63A. In some embodiments, the IL-18 peptide comprises at least three modifications to the amino acid sequence of SEQ ID NO: 1, wherein the modifications comprise E06K, K53A, and S55A; E06K, V11I, and K53A; E06K, C38A, and K53A; or E06K, K53A, and T63A. In some embodiments, the IL-18 peptide comprises at least four modifications to the amino acid sequence of SEQ ID NO: 1, wherein the modifications comprise E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the IL-18 peptide comprises at least six modifications to the amino acid sequence of SEQ ID NO: 1, wherein the modifications comprise E06K, K53A, C38S, C68S, C76S, and C127S; or K53A, T63A, C38S, C68S, C76S, and C127S. In some embodiments, the IL-18 polypeptide comprises at least seven modifications to the sequence of SEQ ID NO: 1, wherein the seven modifications comprise E6K, V11I, C38A, K53A, T63A, C76A, C127A. In some embodiments, the IL-18 polypeptide comprises at least eight modifications to the sequence of SEQ ID NO: 1, wherein the eight modifications comprise E6K, V11I, C38A, K53A, T63A, C68A, C76A, C127A. In some embodiments, the IL-18 peptide comprises at least eight modifications to the amino acid sequence of SEQ ID NO: 1, wherein the modifications comprise Y01G, F02A, E06K, M51G, K53A, D54A, S55A, and T63A. In some embodiments, the IL-18 peptide comprises at least eight modifications to the amino acid sequence of SEQ ID NO: 1, wherein the modifications comprise Y01G, F02A, E06K, M51G, K53A, D54A, S55A, and T63A.

In some embodiments, the IL-18 polypeptide of the fusion immunocytokine comprises E06K and K53A, wherein residue position numbering of the IL-18 polypeptide is based on SEQ ID NO: 1 as a reference sequence. In some embodiments, the IL-18 polypeptide comprises an amino acid sequence at least 80%, at least 85%, at least 90%, at least 95%, or at least 98% identical to the amino acid sequence of SEQ ID NO: 30. In some embodiments, the IL-18 polypeptide comprises an amino acid sequence at least 80%, at least 85%, at least 90%, at least 95%, or at least 98% identical to the amino acid sequence of SEQ ID NO: 59. In some embodiments, the IL-18 polypeptide further comprises an amino acid substitution at one or more cysteine residues. In some embodiments, the IL-18 polypeptide comprises one or more cysteines substituted with either serine or alanine. In some embodiments, the IL-18 polypeptide comprise amino acid substitutions at each cysteine residue of SEQ ID NO: 1. In some embodiments, each cysteine residue is substituted with serine or alanine.

In some embodiments, the IL-18 polypeptide of the fusion immunocytokine comprises a polypeptide sequence having at least about 80%, at least about 85%, at least about 90%, at least about 95%, at least about 98%, at least about 99%, or about 100% sequence identity to SEQ ID NO: 2-73. In some embodiments, the polypeptide sequence is at least about 80% identical to SEQ ID NO: 30. In some embodiments, the polypeptide sequence is at least about 90% identical to SEQ ID NO: 30. In some embodiments, the polypeptide sequence is at least about 95% identical to SEQ ID NO: 30. In some embodiments, the polypeptide sequence is at least about 98% identical to SEQ ID NO: 30. In some embodiments, the polypeptide sequence is identical to SEQ ID NO: 30. In some embodiments, the polypeptide sequence is at least about 80% identical to SEQ ID NO: 59. In some embodiments, the polypeptide sequence is at least about 90% identical to SEQ ID NO: 59. In some embodiments, the polypeptide sequence is at least about 95% identical to SEQ ID NO: 59. In some embodiments, the polypeptide sequence is at least about 98% identical to SEQ ID NO: 59. In some embodiments, the polypeptide sequence is identical to that of SEQ ID NO: 59. In some embodiments, the polypeptide sequence is at least about 80% identical to SEQ ID NO: 73. In some embodiments, the polypeptide sequence is at least about 90% identical to SEQ ID NO: 73. In some embodiments, the polypeptide sequence is at least about 95% identical to SEQ ID NO: 73. In some embodiments, the polypeptide sequence is at least about 98% identical to SEQ ID NO: 73. In some embodiments, the polypeptide sequence is identical to any one of SEQ ID NOs: 68-72. In some embodiments, the polypeptide sequence is at least about 80% identical to any one of SEQ ID NOs: 68-72. In some embodiments, the polypeptide sequence is at least about 90% identical to any one of SEQ ID NOs: 68-72. In some embodiments, the polypeptide sequence is at least about 95% identical to any one of SEQ ID NOs: 68-72. In some embodiments, the polypeptide sequence is at least about 98% identical to any one of SEQ ID NOs: 68-72. In some embodiments, the polypeptide sequence is identical to any one of SEQ ID NOs: 68-72. In some embodiments, the IL-18 polypeptide is one provided in Table 2. In some embodiments, the IL-18 polypeptide is one described in Table 3.

In some embodiments, the end of the IL-18 polypeptide which is fused to the antibody or antigen binding fragment is truncated by one or more amino acids relative to SEQ ID NO: 1. In some embodiments, the IL-18 polypeptide is truncated by 1, 2, 3, 4, 5, 6, 7, 8, 9, 10, or more amino acids.

In certain embodiments, the IL-18 polypeptide of the fusion immunocytokine comprises at least one glycine residue attached to the N-terminus of the IL-18 polypeptide. In certain embodiments, the IL-18 polypeptide comprises a chain of glycine residues attached to the N-terminus of the polypeptide, wherein the chain of glycine residues comprises 1 to 15 glycine residues. In certain embodiments, the IL-18 polypeptide comprises a chain of 1 to 10 glycine residues attached to the N-terminus of the IL-18 polypeptide. In certain embodiments, the IL-18 polypeptide comprises a chain of 1, 2, 3, 4, 5, 6, 7, 8, 9, or 10, or any range therebetween glycine residues attached to the N-terminus of the IL-18 polypeptide. In certain embodiments, the IL-18 polypeptide comprises a glycine residue attached to the N-terminus of the IL-18 polypeptide. In certain embodiments, the IL-18 polypeptide comprises a chain of 2 glycine residues attached to the N-terminus of the IL-18 polypeptide. In certain embodiments, the IL-18 polypeptide comprises a chain of 3 glycine residues attached to the N-terminus of the IL-18 polypeptide. In certain embodiments, the IL-18 polypeptide comprises a chain of 4 glycine residues attached to the N-terminus of the IL-18 polypeptide. In certain embodiments, the IL-18 polypeptide comprises a chain of 5 glycine residues attached to the N-terminus of the IL-18 polypeptide. In certain embodiments, the IL-18 polypeptide comprises a chain of glycine residues attached to the N-terminus of the polypeptide, wherein the chain of glycine residues comprises 1 to 2, 1 to 3, 1 to 4, 1 to 5, 1 to 6, 1 to 7, 1 to 8, 1 to 9, 1 to 10, 1 to 12, 1 to 15, 2 to 3, 2 to 4, 2 to 5, 2 to 6, 2 to 7, 2 to 8, 2 to 9, 2 to 10, 2 to 12, 2 to 15, 3 to 4, 3 to 5, 3 to 6, 3 to 7, 3 to 8, 3 to 9, 3 to 10, 3 to 12, 3 to 15, 4 to 5, 4 to 6, 4 to 7, 4 to 8, 4 to 9, 4 to 10, 4 to 12, 4 to 15, 5 to 6, 5 to 7, 5 to 8, 5 to 9, 5 to 10, 5 to 12, 5 to 15, 6 to 7, 6 to 8, 6 to 9, 6 to 10, 6 to 12, 6 to 15, 7 to 8, 7 to 9, 7 to 10, 7 to 12, 7 to 15, 8 to 9, 8 to 10, 8 to 12, 8 to 15, 9 to 10, 9 to 12, 9 to 15, 10 to 12, 10 to 15, or 12 to 15 glycine residues. In certain embodiments, the IL-18 polypeptide comprises a chain of glycine residues attached to the N-terminus of the polypeptide, wherein the chain of glycine residues comprises 1, 2, 3, 4, 5, 6, 7, 8, 9, 10, 12, or 15 glycine residues. In certain embodiments, the IL-18 polypeptide comprises a chain of glycine residues attached to the N-terminus of the polypeptide, wherein the chain of glycine residues comprises at least 1, 2, 3, 4, 5, 6, 7, 8, 9, 10, or 12 glycine residues. In certain embodiments, the IL-18 polypeptide comprises a chain of glycine residues attached to the N-terminus of the polypeptide, wherein the chain of glycine residues comprises at most 2, 3, 4, 5, 6, 7, 8, 9, 10, 12, or 15 glycine residues.

In addition to the IL-18 polypeptides described herein, additional IL-18 polypeptides which can be incorporated into fusion immunocytokines include those described in, for example, Patent Cooperation Treaty Publication Nos: WO2019051015, WO2022094473, WO2022172944, WO2023010021, WO2023056193, WO2023114829, and WO2023118497.

### Biological Activity

In some embodiments, the fusion immunocytokine exhibits one or more activities associated with the antibody or antigen binding fragment and/or an IL-18 polypeptide.

In some embodiments, the fusion immunocytokine exhibits an ability to bind to the IL-18 receptor. In some embodiments, the fusion immunocytokine exhibits an ability to bind to the IL-18 receptor which is comparable to WT IL-18. In some embodiments, the fusion immunocytokine exhibits an ability to bind to the IL-18 receptor (IL-18Raβ) which is reduced by at most 2-fold, at most 5-fold, at most 10-fold, at most 20-fold, at most 50-fold, at most 100-fold, at most 200-fold, at most 300-fold, at most 400-fold, or at most 1000-fold compared to WT IL-18. In some embodiments, the fusion immunocytokine exhibits an enhanced ability to bind the IL-18Raβ. In some embodiments, the fusion immunocytokine exhibits an ability to bind to the IL-18Raβ which is increased by at least 2-fold, at least 3-fold, at least 5-fold, or at least 10-fold compared to WT IL-18.

In some embodiments, the fusion immunocytokine exhibits an ability to stimulate production of IFNγ upon contact with a cell (e.g., an immune cell, such as an NK cell). In some embodiments, the ability of the fusion immunocytokine to stimulate IFNγ production is somewhat reduced compared to WT IL-18. In some embodiments, a half-maximal effective concentration (EC₅₀) of the ability of the fusion immunocytokine to stimulate production of IFNγ is at most 100-fold higher than, at most 50-fold higher than, at most 20-fold higher than, at most 10-fold higher than, at most 5-fold higher than, or at most 2-fold higher than that of a WT IL-18. In some embodiments, the ability of the fusion immunocytokine to stimulate IFNY production is enhanced compared to WT IL-18. In some embodiments, a half-maximal effective concentration (EC₅₀) of the ability of the fusion immunocytokine to stimulate production of IFNγ is at least 5-fold lower than, at least 10-fold lower than, at least 20-fold lower than, at least 50-fold lower than, at least 75-fold lower than, or at least 100-fold higher than that of a WT IL-18.

In some embodiments, the fusion immunocytokine exhibits an ability to stimulate production of IFNγ upon contact with a cell (e.g., an immune cell, such as an NK cell) which is only somewhat reduced as compared to the IL-18 polypeptide not comprised in the fusion immunocytokine (e.g., the unfused IL-18 polypeptide). In some embodiments, the EC₅₀ of IFNγ stimulation is at most 5-fold greater than, at most 10-fold greater than, at most 50-fold greater than, or at most 100-fold greater than that that of the IL-18 polypeptide not comprised in the fusion immunocytokine. In some embodiments, the fusion immunocytokine exhibits an ability to induce IFNγ production in a cell as measured by half-maximal effective concentration (EC₅₀) which is within about 100-fold of the corresponding IL-18 polypeptide not comprised in the fusion immunocytokine In some embodiments, the fusion immunocytokine exhibits a lower EC₅₀ than WT IL-18. In some embodiments, the fusion immunocytokine exhibits a lower EC₅₀ than WT IL-18 by at least 2-fold, 5-fold, 10-fold, 20-fold, 30-fold, 50-fold, or 100-fold.

In some embodiments, the fusion immunocytokine exhibits a reduced ability to bind IL-18 binding protein (IL-18BP). In some embodiments, the ability of fusion immunocytokine to bind IL-18BP is reduced by at least 2-fold, at least 5-fold, at least 10-fold, at least 20-fold, at least 30-fold, at least 50-fold, or at least 100-fold compared to WT IL-18. In some embodiments, the fusion immunocytokine does not display any substantial ability to bind IL-18 BP.

In some embodiments, the fusion immunocytokine exhibits a reduced ability to have its IFNγ production stimulatory activity inhibited by IL-18BP. In some embodiments, the ability of the fusion immunocytokine to be inhibited by IL-18BP is measured as a half maximal inhibitory concentration (IC₅₀). In some embodiments, the fusion immunocytokine exhibits an IC₅₀ by IL-18BP that is at least 2-fold higher than, at least 5-fold higher than, at least 10-fold higher than, at least 15-fold higher than, at least 20-fold higher than, at least 25-fold higher than, at least 30-fold higher than, at least 40-fold higher than, or at least 50-fold higher than an IC₅₀ of WT IL-18's inhibition by IL-18BP. In some embodiments, the fusion immunocytokine exhibits an IC₅₀ by IL-18BP that is at least 100-fold higher than an IC₅₀ of WT IL-18's inhibition by IL-18BP. In some embodiments, the fusion immunocytokine exhibits an IC₅₀ by IL-18BP that is at least 200-fold higher than an IC₅₀ of WT IL-18's inhibition by IL-18BP. In some embodiments, the fusion immunocytokine exhibits an IC₅₀ by IL-18BP that is at least 500-fold higher than an IC₅₀ of WT IL-18's inhibition by IL-18BP. In some embodiments, the fusion immunocytokine exhibits an IC₅₀ by IL-18BP that is at least 1000-fold higher than an IC₅₀ of WT IL-18's inhibition by IL-18BP.

In some embodiments, the fusion immunocytokine retains binding associated with the antibody or antigen binding fragment. In some embodiments, the fusion immunocytokine retains binding to the antigen of the antibody or antigen binding fragment. In some embodiments, the fusion immunocytokine exhibits binding affinity (K_{D}) to the antigen of the antibody which is within 5-fold of the binding affinity of the antibody not attached to the IL-18 polypeptide. In some embodiments, the fusion immunocytokine exhibits binding affinity (K_{D}) to the antigen of the antibody which is within 2.5-fold of the binding affinity of the antibody not attached to the IL-18 polypeptide. In some embodiments, the binding is determined by ELISA. In some embodiments, the binding is determined by BLI.

In some embodiments, the fusion immunocytokine retains binding to one or more Fc receptors associated with the antibody or antigen binding fragment. In some embodiments, the Fc receptor is selected from FcRn, CD64, CD32a, CD16, and CD32b, or any combination thereof. In some embodiments, the fusion immunocytokine exhibits a binding affinity (K_{D}) to at least one Fc receptor which is within 10-fold of the binding affinity of the antibody not attached to the IL-18 polypeptide. In some embodiments, the fusion immunocytokine exhibits a binding affinity (K_{D}) to at least one Fc receptor which is less than 10-fold higher, less than 5-fold higher, less than 4-fold higher, less than 3-fold higher, less than 2-fold higher, or less than the binding affinity of the antibody not attached to the IL-18 polypeptide. In some embodiments, the fusion immunocytokine exhibits a binding affinity (K_{D}) to each of FcRn, CD64, CD32a, CD16, and CD32B which is less than 10-fold higher, less than 5-fold higher, less than 4-fold higher, less than 3-fold higher, less than 2-fold higher, or less than the binding affinity of the antibody not attached to the IL-18 polypeptide. In some embodiments, the fusion immunocytokine exhibits a binding affinity (K_{D}) to each of FcRn, CD64, CD32a, CD16, and CD32B which is within 10-fold of the binding affinity of the antibody not attached to the IL-18 polypeptide. In some embodiments, the fusion immunocytokine exhibits a binding affinity (K_{D}) to each of FcRn, CD64, CD32a, CD16, and CD32B which is within 20-fold of the binding affinity of the antibody not attached to the IL-18 polypeptide. In some embodiments, the fusion immunocytokine exhibits a binding affinity (K_{D}) to each of FcRn, CD64, CD32a, CD16, and CD32B which is within 50-fold of the binding affinity of the antibody not attached to the IL-18 polypeptide. In some embodiments, the fusion immunocytokine exhibits a binding affinity (K_{D}) to each of FcRn, CD64, CD32a, CD16, and CD32B which is within 100-fold of the binding affinity of the antibody not attached to the IL-18 polypeptide.

In some embodiments, the fusion immunocytokine exhibits synergistic efficacy owing to the presence of both molecules in one molecule. In some embodiments, the fusion immunocytokine exhibits enhanced activity compared to either molecule alone. In some embodiments, the fusion immunocytokine exhibits enhanced anti-tumor growth inhibition compared to the antibody alone. In some embodiments, the fusion immunocytokine exhibits enhanced anti-tumor growth inhibition compared to the antibody and the IL-18 polypeptide administered in combination. In some embodiments, the IL-18 polypeptide is administered as a half-life extended version (e.g., PEGylated, attached to an Fc domain (e.g., an Fc fusion), or attached to a negative control antibody). In some embodiments, the fusion immunocytokine exhibits similar or enhanced antitumor activity at the same concentration as the antibody administered alone. In some embodiments, the fusion immunocytokine exhibits similar or enhanced antitumor activity when administered at a dose which is less that 0.5-fold, 0.25-fold, or 0.1-fold the dose of the antibody alone.

### Compositions

In one aspect, provided herein is a pharmaceutical composition comprising a fusion immunocytokine described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition further comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

In one aspect, described herein is a liquid or lyophilized composition that comprises a described fusion immunocytokine. In some embodiments, the fusion immunocytokine is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The fusion immunocytokines herein can be in a variety of dosage forms. In some embodiments, the fusion immunocytokine is dosed as a reconstituted lyophilized powder. In some embodiments, the fusion immunocytokine is dosed as a suspension. In some embodiments, the fusion immunocytokine is dosed as a solution. In some embodiments, the fusion immunocytokine is dosed as an injectable solution. In some embodiments, the fusion immunocytokine is dosed as an IV solution.

### Methods of Treatment

In one aspect, described herein, is a method of treating cancer in a subject in need thereof, comprising: administering to the subject an effective amount of a fusion immunocytokine or a pharmaceutical composition as described herein. In some embodiments, the cancer is a solid cancer. A cancer or tumor can be, for example, a primary cancer or tumor or a metastatic cancer or tumor. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia

Combination therapies with one or more additional active agents are contemplated herein.

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the fusion immunocytokine is administered in a single dose of the effective amount of immunocytokine, including further embodiments in which (i) the fusion immunocytokine is administered once a day; or (ii) the fusion immunocytokine is administered once a day; or (ii) the fusion immunocytokine is administered to the subject multiple times over the span of one day. In some embodiments, the fusion immunocytokine is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

### Methods of Manufacturing

In one aspect, described herein, is a method of making a fusion immunocytokine, comprising expressing the fusion immunocytokine in a host cell. In some embodiments, the host cell is a bacterial cell, a yeast cell, an insect cell, or a mammalian cell. In some embodiments, expressing the fusion immunocytokine compromises administering to the host cell a nucleic acid encoding the fusion immunocytokine, or a portion thereof. In some embodiments, the fusion immunocytokine is expressed from a single nucleic acid (e.g., a single nucleic acid encoding all relevant polypeptides of the fusion immunocytokine). In some embodiments, the fusion immunocytokine is expressed from multiple nucleic acids (e.g., one nucleic acid encoding a heavy chain and one nucleic acid encoding a light chain, wherein one of the heavy or the light chain is fused to the IL-18 polypeptide).

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof′ and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of IL-18 polypeptides or other polypeptides. As used herein, “attached” or “covalently attached” means that the group is tethered to the indicated reside, and such tethering can include a linking group (i.e., a linker). Thus, for a group “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, Kp is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Throughout the instant description, certain numerical or other similar values may be described as, for example, “at least” or “at most” a set of values indicated in a list form (e.g., “at least 2, 3, 4, 5, or 6”). In such cases, unless context clearly indicates otherwise, it is intended that the phrase “at least,” “at most,” or other similar term is applied individually to each value in the list. For example, the phrase “at least 2, 3, 4, 5, or 6” is to be interpreted as “at least 2, at least 3, at least 4, at least 5, or at least 6.”

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

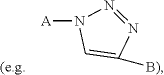

it is intended that the other regioisomer

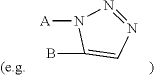

is also encompassed.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Ni is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Ni is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an antibody Fc domain (e.g., an IgG1 Fc domain) (EU numbering). In some embodiments, the desired functionalization is incorporated at residue position 248 of an antibody Fc domain (EU numbering). In some embodiments, position 248 corresponds to the 18th residue in a human IgG CH2 region (EU numbering).

### Sequences (SEQ ID NOS) of IL-18 Polypeptides

TABLE 2

| SEQ | | |
|---|---|---|
| ID NO: | Modification | Sequence |
| 1 | Native | YFGKLESKLS VIRNLNDQVL FIDQGNRPLF EDMTDSDCRD |
| | sequence | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 2 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | E85C | ISFKCMNPPD NIKDTKSDII FFQRSVPGHD NKMQFESSSY |
| | | EGYFLACEKE RDLFKLILKK EDELGDRSIM FTVQNED |
| 3 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKCMNPPD NIKDTKSDII FFQRSVPGHD NKMQFESSSY |
| | C76A, E85C, | EGYFLAAEKE RDLFKLILKK EDELGDRSIM FTVQNED |
| | C127A | |
| 4 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | M86C | ISFKECNPPD NIKDTKSDII FFQRSVPGHD NKMQFESSSY |
| | | EGYFLACEKE RDLFKLILKK EDELGDRSIM FTVQNED |
| 5 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKECNPPD NIKDTKSDII FFQRSVPGHD NKMQFESSSY |
| | C76A, M86C, | EGYFLAAEKE RDLFKLILKK EDELGDRSIM FTVQNED |
| | C127A | |
| 6 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDTKSCII FFQRSVPGHD NKMQFESSSY |
| | C76A, D98C, | EGYFLAAEKE RDLFKLILKK EDELGDRSIM FTVQNED |
| | C127A | |
| 7 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSAENKI |
| | C76A, M86C, | ISFKECNPPD NIKDTKSDII FFQRSVPGHD NKMQFESSSY |
| | C127A | EGYFLAAEKE RDLFKLILKK EDELGDRSIM FTVQNED |
| 8 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDCKSDII FFQRSVPGHD NKMQFESSSY |
| | C76A, T95C, | EGYFLAAEKE RDLFKLILKK EDELGDRSIM FTVQNED |
| | C127A | |
| 9 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | D98C | ISFKEMNPPD NIKDTKSCII FFQRSVPGHD NKMQFESSSY |
| | | EGYFLACEKE RDLFKLILKK EDELGDRSIM FTVQNED |
| 10 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSAENKI |
| | C76A, D98C, | ISFKEMNPPD NIKDTKSCII FFQRSVPGHD NKMQFESSSY |
| | C127A | EGYFLAAEKE RDLFKLILKK EDELGDRSIM FTVQNED |
| 11 | E6K, V11I, | YFGKLKSKLS IIRNLNDQVL FIDQGNRPLF EDMTDSDARD |
| | C38A, K53A, | NAPRTIFIIS MYADSQPRGM AVTISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 12 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 13 | E6K, K53A, | YFGKLKSKLS VIRNLNDQVL FIDQGNRPLF EDMTDSDCRD |
| | T63N | NAPRTIFIIS MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 14 | E6K, K53A, | YFGKLKSKLS VIRNLNDQVL FIDQGNRPLF EDMTDSDCRD |
| | S50A, T63N | NAPRTIFIIA MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 15 | E6K, K53A, | YFGKLKSKLS VIRNLNDQVL FIDQGNRPLF EDMTDSDCRD |
| | S50H, T63N | NAPRTIFIIH MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 16 | E6K, K53A, | YFGKLKSKLS VIRNLNDQVL FIDQGNRPLF EDMTDSDCRD |
| | T63N, S65A | NAPRTIFIIS MYADSQPRGM AVNIAVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 17 | E6K, K53A, | YFGKLKSKLS VIRNLNDQVL FIDQGNRPLF EDMTDSDCRD |
| | S50H | NAPRTIFIIH MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 18 | E6K, C38A, | YFGKLKSKLS VIRNLNDQVL FIDQGNRPLF EDMTDSDARD |
| | K53A, C68A | NAPRTIFIIS MYADSQPRGM AVTISVKAEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 19 | E6K, K53A, | YFGKLKSKLS VIRNLNDQVL FIDQGNRPLF EDMTDSDCRD |
| | K79A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 20 | E6K, K53A, | YFGKLKSKLS VIRNLNDQVL FIDQGNRPLF EDMTDSDCRD |
| | R104A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 21 | E6K, K53A, | YFGKLKSKLS VIRNLNDQVL FIDQGNRPLF EDMTDSDCRD |
| | G108A | NAPRTIFIIS MYADSQPRGM AVTISVKCEK ISTLSCENKI |
| | | ISFKEMNPPD NIKDTKSDII FFQRSVPAHD NKMQFESSSY |
| | | EGYFLACEKE RDLFKLILKK EDELGDRSIM FTVQNED |
| 22 | E6K, K53A, | YFGKLKSKLS VIRNLNDQVL FIDQGNRPLF EDMTDSDCRD |
| | H109A | NAPRTIFIIS MYADSQPRGM AVTISVKCEK ISTLSCENKI |
| | | ISFKEMNPPD NIKDTKSDII FFQRSVPGAD NKMQFESSSY |
| | | EGYFLACEKE RDLFKLILKK EDELGDRSIM FTVQNED |
| 23 | E6K, K53A, | YFGKLKSKLS VIRNLNDQVL FIDQGNRPLF EDMTDSDCRD |
| | K112A | NAPRTIFIIS MYADSQPRGM AVTISVKCEK ISTLSCENKI |
| | | ISFKEMNPPD NIKDTKSDII FFQRSVPGHD NAMQFESSSY |
| | | EGYFLACEKE RDLFKLILKK EDELGDRSIM FTVQNED |
| 24 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A | ISFKEMNPPD NIKDTKSDII FFQRSVPGHD NKMQFESSSY |
| | | EGYFLACEKE RDLFKLILKK EDELGDRSIM FTVQNED |
| 25 | E6K, C38Q, | YFGKLKSKLS VIRNLNDQVL FIDQGNRPLF EDMTDSDQRD |
| | K53A, T63A, | NAPRTIFIIS MYADSQPRGM AVAISVKCEK ISTLSAENKI |
| | C76A | ISFKEMNPPD NIKDTKSDII FFQRSVPGHD NKMQFESSSY |
| | | EGYFLACEKE RDLFKLILKK EDELGDRSIM FTVQNED |
| 26 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSCENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 27 | E6K, C38Q, | YFGKLKSKLS VIRNLNDQVL FIDQGNRPLF EDMTDSDQRD |
| | K53A, T63A, | NAPRTIFIIS MYADSQPRGM AVAISVKCEK ISTLSCENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 28 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 29 | E6K, V11I, | YFGKLKSKLS IIRNLNDQVL FIDQGNRPLF EDMTDSDARD |
| | C38A, K53A, | NAPRTIFIIS MYADSQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 30 | E6K, V11I, | YFGKLKSKLS IIRNLNDQVL FIDQGNRPLF EDMTDSDARD |
| | C38A, K53A, | NAPRTIFIIS MYADSQPRGM AVAISVKCEK ISTLSAENKI |
| | T63A, C76A, | ISFKEMNPPD NIKDTKSDII FFQRSVPGHD NKMQFESSSY |
| | C127A | EGYFLAAEKE RDLFKLILKK EDELGDRSIM FTVQNED |
| 31 | C38A, C76A, | YFGKLESKLS VIRNLNDQVL FIDQGNRPLF EDMTDSDARD |
| | C127A | NAPRTIFIIS MYKDSQPRGM AVTISVKCEK ISTLSAENKI |
| | | ISFKEMNPPD NIKDTKSDII FFQRSVPGHD NKMQFESSSY |
| | | EGYFLAAEKE RDLFKLILKK EDELGDRSIM FTVQNED |
| 32 | C38A | YFGKLESKLS VIRNLNDQVL FIDQGNRPLF EDMTDSDARD |
| | | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 33 | E6K, C38A, | YFGKLKSKLS VIRNLNDQVL FIDQGNRPLF EDMTDSDARD |
| | K53A, T63A | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 34 | E06K, K53A, | YFGKLKSKLS VIRNLNDQVL FIDQGNRPLF EDMTDSDCRD |
| | S55A | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 35 | Y01G, F02A, | GAGKLKSKLS VIRNLNDQVL FIDQGNRPLF EDMTDSDCRD |
| | E06K, M51G, | NAPRTIFIIS GYAAAQPRGM AVAISVKCEK ISTLSCENKI |
| | K53A, D54A, | ISFKEMNPPD NIKDTKSDII FFQRSVPGHD NKMQFESSSY |
| | S55A, T63A | EGYFLACEKE RDLFKLILKK EDELGDRSIM FTVQNED |
| 36 | K53A | YFGKLESKLS VIRNLNDQVL FIDQGNRPLF EDMTDSDCRD |
| | | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 37 | S55A | YFGKLESKLS VIRNLNDQVL FIDQGNRPLF EDMTDSDCRD |
| | | NAPRTIFIIS MYKDAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 38 | E06K | YFGKLKSKLS VIRNLNDQVL FIDQGNRPLF EDMTDSDCRD |
| | | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 39 | E06K, K53A | YFGKLKSKLS VIRNLNDQVL FIDQGNRPLF EDMTDSDCRD |
| | | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 40 | E06K, S55A | YFGKLKSKLS VIRNLNDQVL FIDQGNRPLF EDMTDSDCRD |
| | | NAPRTIFIIS MYKDAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 41 | K53A, S55A | YFGKLESKLS VIRNLNDQVL FIDQGNRPLF EDMTDSDCRD |
| | | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 42 | E06K, K53A, | YFGKLKSKLS VIRNLNDQVL FIDQGNRPLF EDMTDSDCRD |
| | S55A, T63A | NAPRTIFIIS MYADAQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 43 | E06K, K53A, | GFGKLKSKLS VIRNLNDQVL FIDQGNRPLF EDMTDSDCRD |
| | S55A, Y01G | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 44 | E06K, K53A, | YAGKLKSKLS VIRNLNDQVL FIDQGNRPLF EDMTDSDCRD |
| | S55A, F02A | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 45 | E06K, K53A, | YFGKLKSKLS VIRNLNDQVL FIDQGNRPLF EDMTDSDCRD |
| | S55A, D54A | NAPRTIFIIS MYAAAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 46 | E06K, K53A, | YFGKLKSKLS VIRNLNDQVL FIDQGNRPLF EDMTDSDCRD |
| | S55A, M51G | NAPRTIFIIS GYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 47 | C38S, C68S, | YFGKLESKLS VIRNLNDQVL FIDQGNRPLF EDMTDSDSRD |
| | C76S, C127S | NAPRTIFIIS MYKDSQPRGM AVTISVKSEK ISTLSSENKI |
| | | ISFKEMNPPD NIKDTKSDII FFQRSVPGHD NKMQFESSSY |
| | | EGYFLASEKE RDLFKLILKK EDELGDRSIM FTVQNED |
| 48 | C38S, C68S, | YFGKLESKLS VIRNLNDQVL FIDQGNRPLF EDMTDSDSRD |
| | C76S, C127S, | NAPRTIFIIS MYKDSQPRGM AVTISVKSEC ISTLSSENKI |
| | K70C | ISFKEMNPPD NIKDTKSDII FFQRSVPGHD NKMQFESSSY |
| | | EGYFLASEKE RDLFKLILKK EDELGDRSIM FTVQNED |
| 49 | E06K, K53A, | YFGKLKSKLS VIRNLNDQVL FIDQGNRPLF EDMTDSDSRD |
| | S55A, C38S, | NAPRTIFIIS MYADAQPRGM AVTISVKSEC ISTLSSENKI |
| | C68S, C76S, | ISFKEMNPPD NIKDTKSDII FFQRSVPGHD NKMQFESSSY |
| | C127S, K70C | EGYFLASEKE RDLFKLILKK EDELGDRSIM FTVQNED |
| 50 | E06K, K53A, | YFGKLKSKLS VIRNLNDQVL FIDQGNRPLF EDMTDSDCRD |
| | T63A | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 51 | T63A | YFGKLESKLS VIRNLNDQVL FIDQGNRPLF EDMTDSDCRD |
| | | NAPRTIFIIS MYKDSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 52 | E06K, T63A | YFGKLKSKLS VIRNLNDQVL FIDQGNRPLF EDMTDSDCRD |
| | | NAPRTIFIIS MYKDSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 53 | K53A, T63A | YFGKLESKLS VIRNLNDQVL FIDQGNRPLF EDMTDSDCRD |
| | | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 54 | E06K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 55 | K53A, T63A, | YFGKLESKLS VIRNLNDQVL FIDQGNRPLF EDMTDSDSRD |
| | C38S, C68S, | NAPRTIFIIS MYADSQPRGM AVAISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 56 | E6K, K53A, | YFGKLKSKLS VIRNLNDQVL FIDQGNRPLF EDMTDSDSRD |
| | C38S, C76S, | NAPRTIFIIS MYADSQPRGM AVTISVKCEK ISTLSSENKI |
| | C127S | ISFKEMNPPD NIKDTKSDII FFQRSVPGHD NKMQFESSSY |
| | | EGYFLASEKE RDLFKLILKK EDELGDRSIM FTVQNED |
| 57 | E6K, C38S, | YFGKLKSKLS VIRNLNDQVL FIDQGNRPLF EDMTDSDSRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 58 | E6K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 59 | E6K, C38A, | YFGKLKSKLS VIRNLNDQVL FIDQGNRPLF EDMTDSDARD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 60 | E6K, C38Q, | YFGKLKSKLS VIRNLNDQVL FIDQGNRPLF EDMTDSDQRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 61 | E6K, C38A, | YFGKLKSKLS VIRNLNDQVL FIDQGNRPLF EDMTDSDARD |
| | K53A, C76A | NAPRTIFIIS MYADSQPRGM AVTISVKCEK ISTLSAENKI |
| | | ISFKEMNPPD NIKDTKSDII FFQRSVPGHD NKMQFESSSY |
| | | EGYFLACEKE RDLFKLILKK EDELGDRSIM FTVQNED |
| 62 | E6K, C38A, | YFGKLKSKLS VIRNLNDQVL FIDQGNRPLF EDMTDSDARD |
| | K53A, C127A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLAAEKE RDLFKLILKK EDELGDRSIM FTVQNED |
| 63 | E6K, C38A, | YFGKLKSKLS VIRNLNDQVL FIDQGNRPLF EDMTDSDARD |
| | K53A, C76A, | NAPRTIFIIS MYADSQPRGM AVTISVKCEK ISTLSAENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 64 | E6K, K53A, | YFGKLKSKLS VIRNLNDQVL FIDQGNRPLF EDMTDSDARD |
| | C38A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 65 | E6K, C38Q, | YFGKLKSKLS VIRNLNDQVL FIDQGNRPLF EDMTDSDQRD |
| | K53A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 66 | E6K, K53A, | YFGKLKSKLS VIRNLNDQVL FIDQGNRPLF EDMTDSDCRD |
| | K84A | NAPRTIFIIS MYADSQPRGM AVTISVKCEK ISTLSCENKI |
| | | ISFAEMNPPD NIKDTKSDII FFQRSVPGHD NKMQFESSSY |
| | | EGYFLACEKE RDLFKLILKK EDELGDRSIM FTVQNED |
| 67 | E6K, K53A, | YFGKLKSKLS VIRNLNDQVL FIDQGNRPLF EDMTDSDCRD |
| | D98A | NAPRTIFIIS MYADSQPRGM AVTISVKCEK ISTLSCENKI |
| | | ISFKEMNPPD NIKDTKSAII FFQRSVPGHD NKMQFESSSY |
| | | EGYFLACEKE RDLFKLILKK EDELGDRSIM FTVQNED |
| 68 | V11I, C38A, | YFGKLESKLS IIRNLNDQVL FIDQGNRPLF EDMTDSDARD |
| (a.k.a. | M51G, K53A, | NAPRTIFIIS GYADSQPRGM AVTISVKCEK ISTLSAENKI |
| C146) | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 69 | E6K, V11I, | YFGKLKSKLS IIRNLNDQVL FIDQGNRPLF EDMTDSDARD |
| (a.k.a. | C38A, M51G, | NAPRTIFIIS GYADSQPRGM AVAISVKCEK ISTLSAENKI |
| C183) | K53A, T63A, | ISFKEMNPPD NIKDTKSDII FFQRSVPGHD NKMQFESSSY |
| | C76A, C127A | EGYFLAAEKE RDLFKLILKK EDELGDRSIM FTVQNED |
| 70 | N-terminal G, | GYFGKLKSKL SIIRNLNDQV LFIDQGNRPL FEDMTDSDAR |
| (a.k.a. | E6K, V11I, | DNAPRTIFII SGYADSQPRG MAVAISVKCE KISTLSAENK |
| C192) | C38A, M51G, | IISFKEMNPP DNIKDTKSDI IFFQRSVPGH DNKMQFESSS |
| | K53A, T63A, | YEGYFLAAEK ERDLFKLILK KEDELGDRSI MFTVQNED |
| | C76A, C127A | |
| 71 | N-terminal G, | GYFGKLKSKL SIIRNLNDQV LFIDQGNRPL FEDMTDSDAR |
| (a.k.a. | E6K, V11I, | DNAPRTIFII SMYADSQPRG MAVAISVKCE KISTLSAENK |
| C141) | C38A, K53A, | IISFKEMNPP DNIKDTKSDI IFFQRSVPGH DNKMQFESSS |
| | T63A, C76A, | YEGYFLAAEK ERDLFKLILK KEDELGDRSI MFTVQNED |
| | C127A | |
| 72 | N-terminal | GGGGYFGKLK SKLSIIRNLN DQVLFIDQGN RPLFEDMTDS |
| (a.k.a. | 4xG, E6K, | DARDNAPRTI FIISMYADSQ PRGMAVAISV KCEKISTLSA |
| C140) | V11I, C38A, | ENKIISFKEM NPPDNIKDTK SDIIFFQRSV PGHDNKMQFE |
| | K53A, T63A, | SSSYEGYFLA AEKERDLFKL ILKKEDELGD RSIMFTVQNE D |
| | C76A, C127A | |
| 73 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDTKSDII FFQRSVPGHD NKMQFESSSY |
| | C76A, C127A | EGYFLAAEKE RDLFKLILKK EDELGDRSIM FTVQNED |

### Additional Exemplary II-18 Constructs

Also provided herein are IL-18 polypeptides which comprise the modifications to SEQ ID NO: 1 listed in the table below, each of which is assigned a Composition ID, which can be incorporated into a fusion immunocytokine as provided herein. In some embodiments, the IL-18 polypeptide of a fusion immunocytokine comprises the set of amino acid substitutions shown for any one of the constructs depicted below. In the constructs depicted below, each of the substitutions is listed using SEQ ID NO: 1 as a reference sequence. In some embodiments, the IL-18 polypeptide a fusion immunocytokine comprises only the substitutions shown for a construct below relative to SEQ ID NO: 1 (i.e., the IL-18 polypeptide has only the indicated set of substitutions and the remaining residues are those set forth in SEQ ID NO: 1).

TABLE 3
Additional IL-18 Polypeptides

| Composition ID/ | | Composition ID/ | | Composition ID/ | |
|---|---|---|---|---|---|
| Substitutions to | | Substitutions to | | Substitutions to | |
| SEQ ID NO: 1 | | SEQ ID NO: 1 | | SEQ ID NO: 1 | |
| C143 | V11I, C38A, | C156 | V11I, C38A, | C168 | V11I, C38A, |
| | K53A, C76A, | | N41A, K53A, | | C76A, S105K, |
| | C127A | | C76A, C127A | | C127A |
| C144 | V11I, C38A, | C157 | V11I, C38A, | C174 | K8L, E6K, |
| | K53A, T63A, | | K53A, C76A, | | V11I, C38A, |
| | C76A, C127A | | C127A, D132A | | K53A, T63A, |
| | | | | | C76A, C127A |
| C145 | V11I, C38A, | C158 | V11I, C38A, | C175 | E6K, V11I, |
| | K53A, S55A, | | K53A, C76A, | | C38A, I49E, |
| | C76A, C127A | | G108A, C127A | | K53A, T63A, |
| | | | | | C76A, C127A |
| C147 | V11I, C38A, | C159 | V11I, C38A, | C176 | E6K, V11I, |
| | K53A, D54A, | | K53A, C76A, | | C38A, I49M, |
| | C76A, C127A | | H109A, C127A | | K53A, T63A, |
| | | | | | C76A, C127A |
| C148 | F2A, V11I, | C160 | V11I, C38A, | C177 | E6K, V11I, |
| | C38A, K53A, | | K53A, C76A, | | C38A, I49R, |
| | C76A, C127A | | D110A, C127A | | K53A, T63A, |
| | | | | | C76A, C127A |
| C149 | V11I, E31A, | C161 | K8R, V11I, | C178 | E6K, V11I, |
| | C38A, K53A, | | C38A, C76A, | | C38A, K53A, |
| | C76A, C127A | | Q103E, C127A | | T63A, C76A, |
| | | | | | Q103R, C127A |
| C150 | V11I, T34A, | C162 | K8E, V11I, | C179 | E6K, K8E, |
| | C38A, K53A, | | C38A, C76A, | | V11I, C38A, |
| | C76A, C127A | | Q103R, C127A | | K53A, T63A, |
| | | | | | C76A, Q103R, |
| | | | | | C127A |
| C151 | V11I, D35A, | C163 | V11I, C38A, | C180 | E6K, V11I, |
| | C38A, K53A, | | C76A, Q103K, | | C38A, K53A, |
| | C76A, C127A | | C127A | | T63A, C76A, |
| | | | | | C127A, V153R |
| C152 | V11I, S36A, | C164 | V11I, C38A, | C181 | E6K, V11I, |
| | C38A, K53A, | | S55H, C76A, | | C38A, K53A, |
| | C76A, C127A | | C127A | | T63A, C76A, |
| | | | | | C127A, V153E |
| C153 | V11I, D37A, | C165 | V11I, C38A, | C182 | E6K, V11I, |
| | C38A, K53A, | | S55R, C76A, | | C38A, K53A, |
| | C76A, C127A | | C127A | | T63A, C76A, |
| | | | | | C127A, V153Y |
| C154 | V11I, E31A, | C166 | V11I, C38A, | C184 | E6R, V11I, |
| | D37A, C38A, | | S55T, C76A, | | C38A, K53A, |
| | K53A, C76A, | | C127A | | T63A, C76A, |
| | C127A | | | | C127A |
| C155 | V11I, C38A, | C167 | V11I, C38A, | C142 | Y1M, E6K, |
| | D40A, K53A, | | C76A, S105I, | | V11I, C38A, |
| | C76A, C127A | | C127A | | K53A, T63A, |
| | | | | | C76A, C127A |
| CX90 | M51K, K53S, | CX91 | C38S, M51K, | CX92 | M3L, C38S, |
| | Q56L, P57A, | | K53S, Q56L, | | C68S, C76S, |
| | M60L, S105D, | | P57A, M60L, | | C127S, E6A, |
| | D110S, N111R | | C68D, S105D, | | K53A |
| | | | D110S, N111R | | |
| CX93 | C38M, C68S, | CX94 | C38S, C68S, | CX95 | M51A, K53G, |
| | C76S, C127S, | | C76S, C127S, | | Q56R, P57A, |
| | E6A, K53A | | E6Q, S10C, | | M60K |
| | | | K53D, N111T, | | |
| | | | N155C | | |
| CX96 | C38M, C76S, | CX97 | C38S, C76S, | CX98 | M3L, C38S, |
| | C127S, E6A, | | C127S, E6Q, | | C76S, C127S, |
| | K53A | | S10C, K53D, | | E6A, K53A |
| | | | N111T, N155C | | |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### Examples

### Example 1A-Recombinant IL18 Expression and Purification

Recombinant IL-18 variants suitable for linking to an antibody or antigen binding fragment as provided herein can be prepared according to the protocols provided below. In some instances, the recombinant IL-18 will contain a cysteine residue at the desired point of attachment of the linker, or may include an unnatural amino acid (e.g., azidolysine) suitable for attachment of the linker at the desired point of attachment.

Soluble his-SUMO-IL18 Variants

E. coli BL21 (DE3) harboring a plasmid encoding a N-His-SUMO tagged IL-18 variant fusion is inoculated into 3 L LB culture medium and induced with 0.4 mM IPTG at 30° C. for 6h. Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Fractions containing the protein are pooled, dialyzed into PBS pH 7.4 and followed by SUMO digestion. Then the protein is two-step purified with Ni-NTA beads (continue with flow through sample) and gel filtration. Fractions containing the protein are pooled and QC is performed using analytical techniques, such as SDS-PAGE and analytical SEC.

Insoluble his-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6h. Cells are pelleted and cell lysis is done by sonication in lysis buffer: PBS, 8 M urea, pH 7.4. Protein is purified via Ni-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Ni-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl).

### Example 1B-Additional Methods for Recombinant IL18 Expression and Purification

The following protocols were also used to prepare certain IL-18 polypeptides provided herein which were subsequently used either in assays for conversion into immunocytokine compositions (i.e., chemically conjugated to antibodies or antigen binding fragments) as provided herein.

### Expression of IL-18 Polypeptides

IL-18 polypeptide were produced as an N-terminal fusion to N-His-SUMO-IL18. The gene was synthesized and cloned by a commercial vendor. Plasmids were transformed into E. coli BL21 (DE3). Expression was performed in shake flasks with TB medium. The cells were grown at 37° C. until an OD600 of approximately 1.2 was reached, after which they were induced by 0.1 mM IPTG and cultured for another 20 hours at 18° C. Cells were harvested by centrifugation.

### Purification of IL18 Polypeptides

Cell lysis-Cells were resuspended in lysis buffer (20 mM Tris/HCl, pH 8.0, 0.15 M NaCl, 10 mM Imidazole, 1 tablet of EDTA-free complete protease inhibitor (Roche, COEDTAF-RO) per liter production) at 100 mL buffer/L culture and disrupted twice with a homogenizer at 1000 bar. The lysate was cleared of debris by centrifugation at 40′000 g for 2x 45 minutes, changing flask in between, and subsequent filtration through a 0.22 μm filter.

Affinity Purification and Endotoxin Removal-The lysate was loaded on Ni NTA resin (Cytiva, 17524802) pre-equilibrated with 20 mM Tris/HCl, pH 8.0, 0.15 M NaCl, 10 mM Imidazole, at 5 mL/min and washed with the same buffer for 5 CV. To remove endotoxins, the column was washed with 20 mM Tris/HCl, pH 8.0, 0.15 M NaCl, 10 mM Imidazole, 0.1% Tryton X-114 at 10 mL/min for 30 CV. The column was washed with 20 mM Tris/HCl, pH 8.0, 0.15 M NaCl, 10 mM Imidazole, for 5 CV at 5 mL/min and the protein of interest eluted by linear increase of imidazole concentration. The column was then regenerated by 0.5M NaOH.

SUMO digestion and dialysis-To cleave the SUMO tag, SUMO protease was added to the elution pool at a w/w ratio of 1:250 (protein: SUMO enzyme) and incubated for 18 hours at 4° C. At the same time, the protein was dialysed (20 mM Tris, pH 8.0, 150 mM NaCl), to reduce the imidazole concentration.

Purification by reverse IMAC-In order to remove the cleaved tag and the SUMO protease, the digested protein was flown through a Ni NTA resin column pre-equilibrated with 20 mM Tris/HCl, pH 8.0, 0.15 M NaCl, 10 mM Imidazole, at 5 mL/min. The flow-through was collected.

Buffer Exchange-The flow-through was concentrated to 2.6 mg/mL and buffer exchanged into either 20 mM HEPES, 150 mM NaCl, 0.5 mM TCEP, 10% glycerol, pH7.5 or PBS, 10% glycerol, pH7.4. Proteins were stored at −70° C. until further quality controls.

### Example 2-Generation of Fusion Immunocytokines

The fusion immunocytokines described herein can be prepared using many techniques known in the art for the expression, purification, and manufacturing if fusion proteins, including those discussed above.

### Example 3-Generation of Conjugated IL-18 Immunocytokines

In addition to the fusion immunocytokines provided herein, immunocytokines of IL-18 can also be prepared by chemical conjugation to a suitable antibody such as those provided herein (e.g., using AJICAP™ technology). Below is an exemplary protocol which can be used to prepare such immunocytokines, of which an analogous protocol was used to prepare such conjugated immunocytokines which are described in more detail below (Compositions A-F, each of which comprise a single molecule of IL-18 polypeptide linked to residue K248 of the Fc region of the relevant antibody (EU numbering).

### Conjugation of IL-18 Polypeptide With Bifunctional Linking Group

An IL-18 polypeptide as provided herein can conjugated to a bifunctional linking group prior to forming the full linker of conjugated immunocytokine. In some cases, the bifunctional linking group first attaches to a desired residue of the IL-18 polypeptide at the point of attachment of the linker (e.g, residue C68 of the IL-18 polypeptide). Once attached to the IL-18 polypeptide, the second functionality of the bifunctional linking group is used to attach to a second portion. An exemplary protocol on an IL-18 polypeptide with a cysteine residue point of attachment (e.g., C68, such as that of an IL-18 polypeptide of SEQ ID NO: 30) provided herein is described below.

Conjugation-The IL-18 polypeptide is stored at a concentration of 2.4 mg/mL at −80° C. in potassium phosphate buffer (pH 7.0) containing 50 mM KCl and 1 mM DTT. The sample is thawed on ice yielding a clear solution. The protein solution is diluted in PBS, pH 7.4. A clear solution is obtained at a concentration of ˜ 0.4 mg/mL.

The protein solution is dialyzed against PBS, pH 7.4 (twice against 600 mL for 2 h and once against 800 mL for 18 h). After dialysis, a clear solution is obtained with no sign of precipitation. Protein concentration is obtained using UV absorbance at 280 nm and by BCA protein assay.

A stock solution of bi-functional linking group (e.g., bromoacetamido-PEG5-azide, CAS: 1415800-37-1) in water is prepared at a concentration of 20 mM. 500 μL of the protein solution are mixed with 25 μL of linking group solution. pH was adjusted to 7.5 and it was let to react for 3 h at 20° C.

The progress of the synthesis is monitored by reverse-phase HPLC using a gradient of 5 to 30% (2.5 min) and 30 to 75% (7.5 min) CH3CN with 0.1% TFA (v/v) on a Aeris WIDEPORE C18 200 Å column (3.6 μm, 150×4.6 mm) at a flow rate of 1 mL/min at 40° C. and by MALDI-TOF MS.

Purification-In some cases, ion-exchange chromatography is used to purify the conjugated protein. To remove the excess of probe, the reaction mixture (volume is around 500 μL) is flowed through a Hi-Trap-G-FF-1 mL column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl, 5% glycerol) and concentrated at 0.4 mg/mL. The concentration of purified protein is determined by UV absorbance at 280 nm and by BCA protein assay. The protein solution is kept at −80° C.

Characterization-The purity and identity of the recombinant protein from commercial source and the conjugated protein is confirmed by aSEC, HPLC and MALDI-TOF MS

### Preparation of a Conjugatable Antibody

A modified antibody (e.g., an anti-PD-1 antibody such as nivolumab or LZM-009) comprising a DBCO conjugation handle is prepared using a protocol modified from Examples 2-4 of US Patent Publication No. US2020019165A1. Briefly, the antibody with a free sulfhydryl group attached to a lysine residue side chain in the Fc domain is prepared by reacting the antibody with an affinity peptide configured to deliver a protected version of the sulfhydryl group (e.g., a thioester) to the lysine residue. The protecting group is then removed to reveal the free sulfhydryl. The free sulfhydryl is then reacted with a bifunctional reagent comprising a bromoacetamide group connected to the DBCO conjugation handle through a linking group

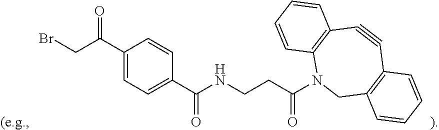

The method can be used to produce an antibody with one DBCO group present (DAR1) and/or two DBCO groups attached to the antibody (DAR2, one DBCO group linked to each Fc of the antibody).

### Conjugation of Antibody to IL-18 Polypeptide

The DBCO modified antibody is then conjugated to a IL-18 polypeptide comprising an azide group at a desired point of attachment (e.g., an IL-18 polypeptide which contains an amino acid with an azide side chain or an IL-18 linked to an azide using a bifunctional linking group as in Example 4). DBCO modified antibody with one (DAR1) or two (DAR2) reactive handles are reacted with 2-10 equivalents of azide containing IL-18 (pH 5.2 buffer, 5% trehalose, rt, 24 h). In an alternative embodiment, antibody comprising two DBCO conjugation handles is reacted either as an excess reagent (e.g., 5-10 equivalents) with 1 equivalent of IL-18 comprising an azide functionality to produce a DAR1 antibody or the antibody comprising two DBCO conjugation handles is reacted with 1 equivalent of antibody with excess reagent of IL-18 comprising an azide (e.g., 5-10 equivalents) to produce a DAR2 antibody.

### Purification and Characterization of Antibody-IL-18 Immunocytokine

The resulting immunocytokine is purified by cation-exchange chromatography and/or size exclusion chromatography to obtain purified immunocytokine. Antibody-IL-18 polypeptide immunocytokine is purified from unreacted IL-18 and aggregates using a desalting column, CIEX and SEC (GE Healthcare Life Sciences AKTA pure, mobile phase: Histidine 5.2/150 mM NaCl/5% Trehalose, column: GE Healthcare Life Sciences SUPERDEX™ 200 increase 3.2/300, flow rate: 0.5 mL/min).

The purity and identity of the antibody-IL-18 polypeptide immunocytokine is confirmed by RP-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN: IPA: ETOH: H2O (5:1.5:2:1.5), flow rate: 0.5 mL/min, injection amount: 10 μg (10 μL Injection of 1 mg/mL), gradient: 0% to 20% mobile phase B in 50 min) and SDS-PAGE.

### Example 4-Characterization of Immunocytokine IL-18 Activity

The ability of the immunocytokines to perform various IL-18 activities is measured as provided below, as well as relevant comparisons to non-conjugated IL-18 polypeptides. Fusion immunocytokines as described herein are also determined, and it is predicted that they will exhibit similar properties to those of the conjugate immunocytokines for which data is provided herein.

### Surface Plasmon Resonance

The interaction of immunocytokines, wild type IL-18, and of IL-18 polypeptides with human IL-18 receptor subunits are measured with Surface Plasmon Resonance (SPR) technology. Anti-human IgG antibodies are bound by amine coupling onto a CM5 chip to capture 6 μg/mL of Fc fused human IL-18Ra, 6 μg/mL of Fc fused human IL-18RB, or 2 μg/mL of Fc fused human IL-18BP isoform a (IL-18BPa) for 30 min before capture. In other settings, 6 μg/mL of alpha and beta IL-18 receptors are mixed and pre-incubated for 30 min before capture of the alpha/beta heterodimer IL-18 receptor.

The kinetic binding of the IL-18 analytes and immunocytokines are measured with a Biacore 8K instrument in two-fold serial dilutions starting at 1 uM down to 0.98 nM. Regeneration of the surface back to amine coupled anti IgG antibody is done after every concentration of analyte. To measure the protein association to the receptors, the samples are injected with a flow rate of 50 μL/min for 60 s, followed by 300 s buffer only to detect the dissociation. The used running buffer is 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogenous ligand fit model is applied for the alpha/beta heterodimer binding.

IL-18BP Binding alphaLISA Assay

A human IL-18BP AlphaLISA Assay Kit is used to determine the binding affinity of each immunocytokine and IL-18 variant for IL-18BP, which detected the presence of free form IL-18BP.

Sixteen three-fold serial dilutions of IL-18 analytes are prepared in aMEM medium supplemented with 20% FCS, Glutamax, and 25 UM B-mercaptoethanol in the presence of 5 ng/ml of His-tagged human IL-18BP. Final IL-18 analytes concentration range from 2778 nM to 0.2 μM.

After 1 hr incubation at room temperature, free IL-18BP levels are measured using a Human IFNγ AlphaLISA Assay Kit. In a 384 well OPTIplate, 5 μL of 5X Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2X streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (K_{D}) is calculated based on a variable slope, four parameter analysis using GraphPad PRISM software.

### IFNγ Induction Cellular Assay

The ability of immunocytokines and IL-18 polypeptides provided herein are assessed for ability to induce IFNγ in a cellular assay according to the protocol below.

The NK cell line NK-92 derived from a patient with lymphoma (ATCC® CRL-2407™) is cultured in aMEM medium supplemented with 20% FCS, Glutamax, 25 UM B-mercaptoethanol, and 100 IU/mL of recombinant human IL-2.

On the day of experiment, cells are harvested and washed with aMEM medium without IL-2 and containing 1 ng/ml of recombinant human IL-12. After counting, cells are seeded at 100,000 cells/well in a 384 well titer plate and incubated at 37° C./5% CO₂. Sixteen 4-fold serial dilutions of IL-18 analytes are prepared in aMEM medium, and 1 ng/ml of IL-12 were added to the NK-92 cells. Final IL-18 analyte concentrations range from 56 nM to 5x10-5 μM.

After incubating the cells for 16-20 hr at 37° C./5% CO₂, 5 μL of supernatant is carefully transferred to a 384 microwell OptiPlate. IFNγ levels are measured using a human IFNY AlphaLISA Assay Kit. Briefly, 10 μL of 2.5X AlphaLISA Anti-IFNγ acceptor beads and biotinylated antibody anti-IFNγ mix are added to the 5 μL of NK-92 supernatants. The mixtures are incubated for 1 hr at room temperature with shaking. Under subdued light, 2.5 μL of 2X streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC₅₀) are calculated based on a variable slope and four parameter analysis using GraphPad PRISM software.

### IL-18 Binding Protein Inhibition Cellular Assay

The NK cell line NK-92 derived from a patient with lymphoma (ATCC® CRL-2407™) is cultured in aMEM medium supplemented with 20% FCS-Glutamax, 25 μM B-mercaptoethanol, and 100 IU/mL of recombinant human IL-2.

On the day of experiment, cells are harvested and washed with aMEM medium without IL-2 and containing 1 ng/ml of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in aMEM medium. 1 ng/ml of IL-12 containing 2 nM of each IL-18 polypeptide variant is added to the NK-92 cells. The final IL-18 analyte concentration is 1 nM, and the final IL-18BPa concentration ranged from 566 nM to 17 μM.

After incubating the cells for 16-20 hr at 37° C./5% CO₂, 5 μL of the supernatant is carefully transferred to a 384 microwell OptiPlate. IFNγ levels are measured using a human IFNγ AlphaLISA Assay Kit. Briefly, 10 μL of 2.5X AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2X SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC₅₀) are calculated based on a variable slope and four parameter analysis using GraphPad PRISM software.

### IFNγ Induction on Primary Human Cells

Ability of IL-18 variants to stimulate Human peripheral blood mononuclear cells (PBMCs) was assessed according to the following protocol.

Isolation of lymphocytes: Blood from Buffy Coats of healthy volunteers was diluted with equal volume of PBS and slowly poured on top of SepMate tube prefilled with 15 mL Histopaque-1077. Tubes were centrifuged for 10 minutes at 1200g, the top layer was collected and washed 3 times with PBS containing 2% of Fetal Bovine Serum. PBMCs were counted and cryopreserved as aliquots of 20× 10⁶ cells.

Cryopreserved PBMCs were thawed and seeded at 150 000 cells/well in a 96w round bottom 96 well plate. PBMCs were stimulated with a gradient of human IL-18 variants ranging from 0.2 pg/mL to 3600 ng/mL. All stimulations were performed in the presence of hIL-12 (1 ng/ml, Sino Biological, #CT011-H08H) for 24 hrs in RPMI containing 10% Fetal Bovine Serum.

Cytokine production after 24 hr stimulation were measured using Legendplex bead-based cytokine assay (Biolegend #740930) according to manufacturer protocol. Half maximal effective concentrations (EC₅₀) of IFNg released in culture supernatant were calculated based on a variable slope and four parameter analysis using GraphPad PRISM software.

IFNγ Induction on primary mouse cells

Ability of IL-18 variants to stimulate murine splenocytes was assessed according to the following protocol.

Cryopreserved splenocytes isolated from BALB/c and C57BL6 mice were purchased from IQ Biosciences (Berkeley, CA, USA).

Cryopreserved splenocytes were thawed, treated with DNAseI, and seeded at 200 000 cells/well in a 96w round bottom 96 well plate. Splenocytes were stimulated with a gradient of human IL-18 variants ranging from 0.2 pg/mL to 3600 ng/mL. All stimulations were performed in the presence of mIL-12 (1 ng/ml, Peprotech, cat #210-12) for 24 hrs in RPMI containing 10% Fetal Bovine Serum.

Cytokine production after 24 hr stimulation were measured using Legendplex bead-based cytokine assay (Biolegend #740622) according to manufacturer protocol. Half maximal effective concentrations (EC₅₀) of IFNg released in culture supernatant were calculated based on a variable slope and four parameter analysis using GraphPad PRISM software.

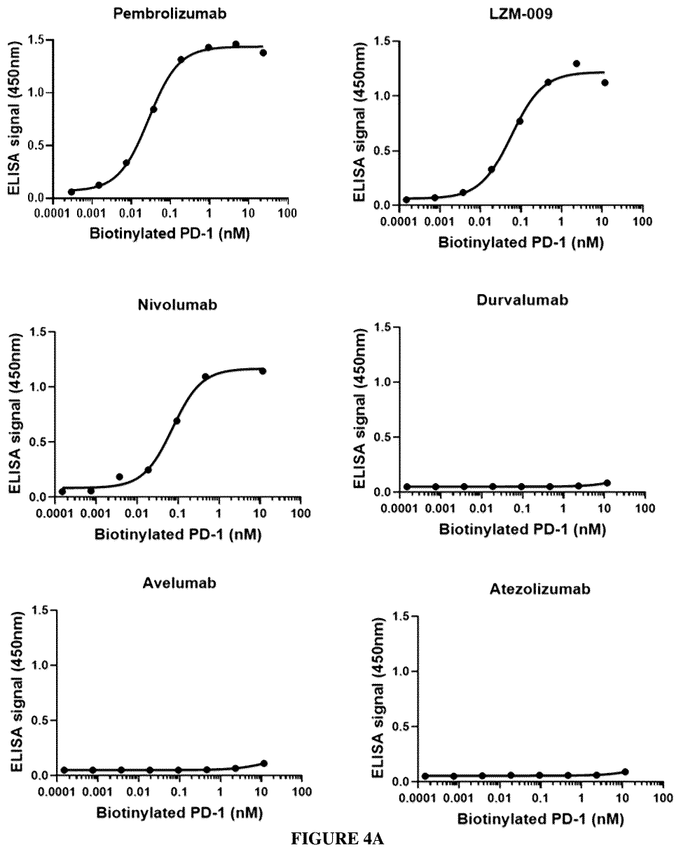

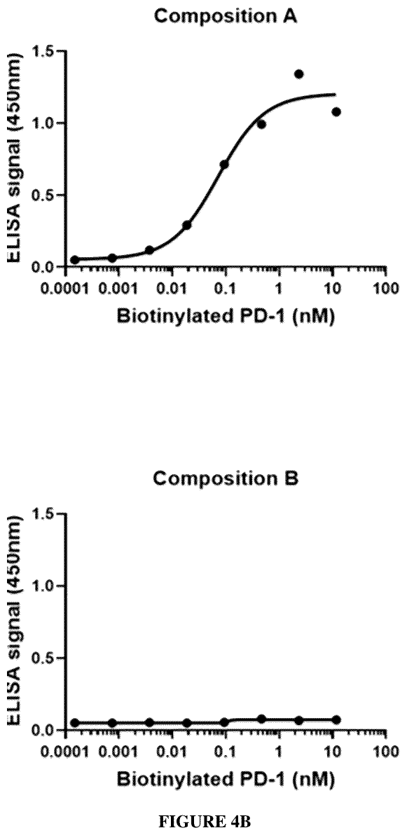

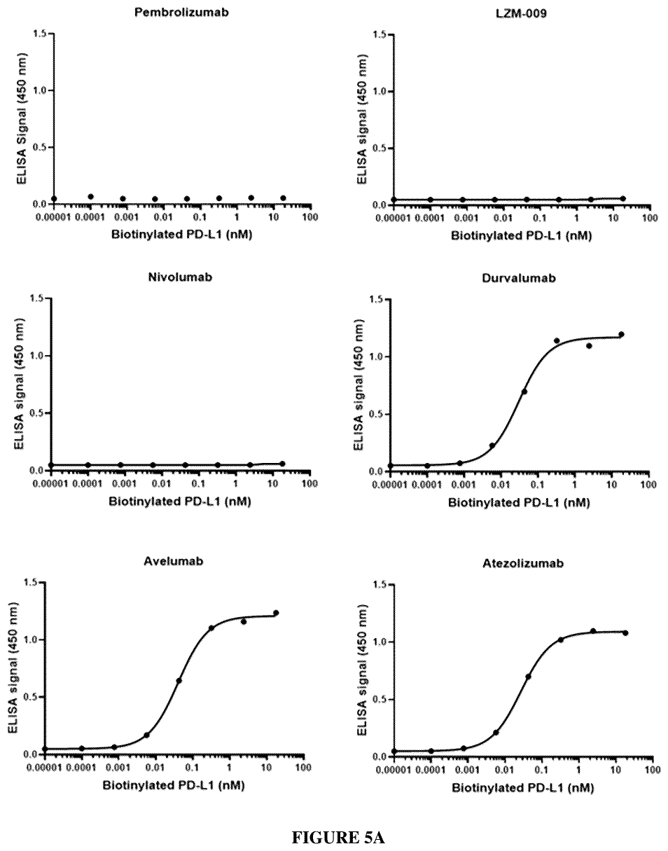

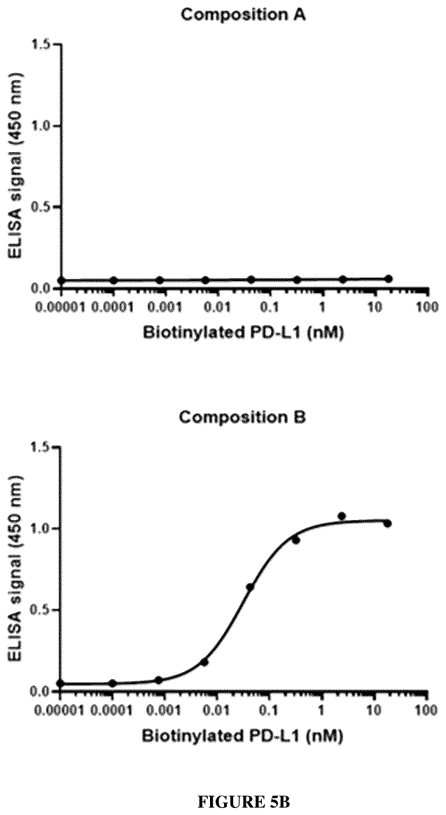

### Example 5-Immune Cell Associated Antigen Binding ELISA Assay of Conjugated Immunocytokines (FIG. 4A, FIG. 4B, FIG. 5A, and FIG. 5B)

The interaction of the unmodified antibodies and corresponding IL-18 immunocytokines with relevant immune cell associated antigen are measured by ELISA assay. For these studies, Corning high-binding half-area plates (Fisher Scientific, Reinach, Switzerland) are coated overnight at 4° C. with 25 ul of unmodified antibodies corresponding IL-18 immunocytokines at 5 μg/ml in PBS. Plates are then washed four times with 100 ul of PBS-0.02% Tween20. Plate surfaces are blocked with 25 ul of PBS-0.02% Tween20-1% BSA at 37° C. during 1h. Plates are then washed four times with 100 ul of PBS-0.02% Tween20. Twenty-five microliters (25 ul) of recombinant biotinylated human PD-1 (Biotinylated Recombinant Human PD-1/CD279-Fc Chimera,carrier-free, Biolegend #789406) or PD-L1 (Biotinylated Human PD-L1/B7-H1, ACROBiosystems, PD1-H82E5-25UG) protein are added in seven-fold serial dilutions starting at 12 nM down to 0.15 μM into PBS-0.02% Tween20-0.1% BSA and incubated at 37° C. during 2h. Plates are then washed four times with 100 ul of PBS-0.02% Tween20. Twenty-five microliters of Streptavidin-Horseradish peroxidase (#RABHRP3, Merck, Buchs, Switzerland) diluted at 1:500 into PBS-0.02% Tween20-0.1% BSA are added to each well and incubated at Room Temperature during 30 min. Plates are then washed four times with 100 ul of PBS-0.02% Tween20. Fifty microliters of TMB substrate reagent (#C_{L}07, Merck, Buchs, Switzerland) are added to each well and incubated at 37° C. during 5 min. After 5 min at 37° C., Horseradish peroxidase reaction is stopped by adding 50 ul/well of 0.5M H2SO₄ stop solution. ELISA signal is then measured at 450 nm on an ENSPIRE® plate reader from Perkin Elmer (Schwerzenbach, Switzerland). Results from this experiment are shown in the table below.

KD values of the interaction of immunocytokines
with PD-1 and PD-L1 as measured by ELISA

| Compo- | | IL-18 | PD-1 KD | PD-L1 KD |
|---|---|---|---|---|
| sition | Antibody | polypeptide | (pM) | (pM) |
| — | Pembrolizumab/ | — | | >100000 |
| | Keytruda | | | |
| | LZM-009 | — | 40.7 | >100000 |
| — | Nivolumab/Opdivo | — | 91.9 | >100000 |
| — | Durvalumab/Imfinzi | — | >100000 | 146 |
| — | Atezolizumab/ | — | >100000 | 443 |
| | Tecentriq | | | |
| — | Avelumab/Bavencio | — | >100000 | 36 |
| A | LZM-009 | SEQ ID NO: | 46.7 | >100000 |
| | | 30 | | |
| B | Atezolizumab/ | SEQ ID NO: | >100000 | 473 |
| | Tecentriq | 30 | | |

FIG. 4A and FIG. 4B show plots measuring ability of the unmodified and of conjugated anti-PD1 antibodies to bind with human PD1/CD279 ligand, with the figure showing ELISA signal on the y-axis and dosage of the biotinylated PD-1 protein on the x-axis. The unconjugated reference antibodies are Pembrolizumab, LZM-009, Nivolumab, Atezolizumab, Durvalumab, and Avelumab. The conjugated antibodies tested in this figure are compositions A and composition B. It is predicted that IL-18 fusion immunocytokines will display similar results.

FIG. 5A and FIG. 5B show plots measuring ability of the unmodified and of conjugated antibodies to bind with human PD-L1/B7-H1 ligand, with the figure showing ELISA signal on the y-axis and dosage of the biotinylated PD-L1 protein on the x-axis. The unconjugated reference antibodies are Pembrolizumab, LZM-009, Nivolumab, Atezolizumab, Durvalumab, and Avelumab. The conjugated antibodies tested in this figure are compositions A and composition B. It is predicted that IL-18 fusion immunocytokines will display similar results.

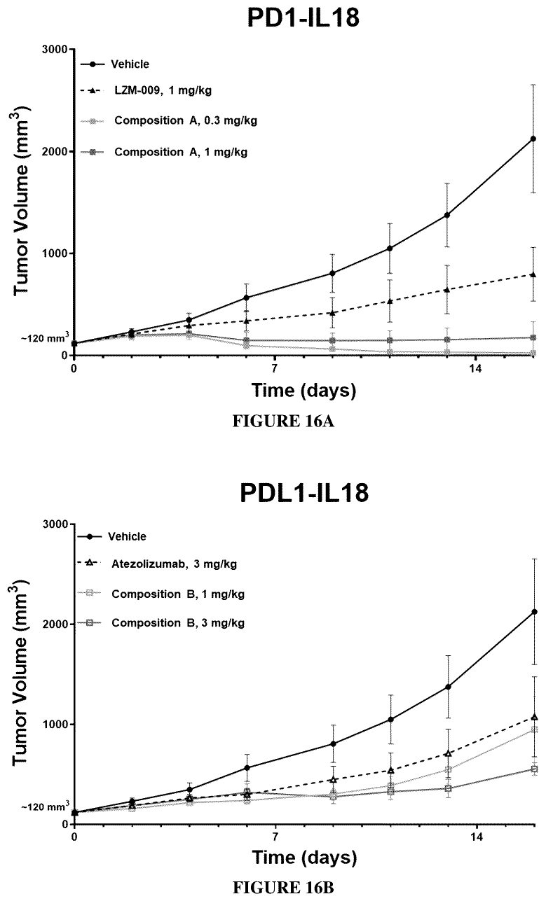

### Example 6-Kinetic Analysis of Binding of reference antibodies and Conjugated Immunocytokines to Immune Cell Associated Antigens (FIG. 16)

Based on Bio-Layer Interferometry (BLI), Octet® BLI systems enable real-time, label-free analysis for the determination of kinetics and affinity of a ligands to its receptor. Here anti-human IgG FC Capture (AHC) sensors are loaded with the test items (ICs). Sensors are first dipped into a kinetic buffer for baseline measurement, then into an analyte solution, here human PD1, to allow association and again into a buffer solution where the analyte is allowed to come off the ligand (dissociation). Several concentrations of analyte are run in parallel and enable the calculation of affinity parameters: Ka, Kd, KD.

Typically, first, the sensors are regenerated by 3 cycles of dipping into 10 mM glycine solution at pH=2 for 20 seconds, followed by 20 second kinetics buffer and a final 60 seconds in kinetics buffer to establish the initial signal (baseline). Second, the loading column will contain the ligand, here the unmodified PD-1 antibodies and of IL-18 polypeptide conjugated PD-1 antibody, at a fixed concentration determined in the loading scout experiment (20 μg/mL). Then another wash/baseline step allows non immobilized proteins to be washed away. The association column will contain the 2-fold dilution series of the analyte (His-tagged human PD1, R&D #8986-PD) including a no analyte control. The highest concentration should be ˜10-fold the KD. The dissociation designates the sensors to return to previous baseline column with kinetics buffer. After acquisition, the data is analyzed with Data Analysis Studio software (Sartorius). Data sets are first preprocessed by subtracting references samples and aligning curves on the baseline step. Group fitting is then applied to the data series and kinetics parameters are calculated. Results from this experiment are shown in the table below.

Binding kinetics of the interaction of reference
antibodies and immunocytokines with PD-1 as
measured by Bio-Layer Interferometry (BLI)

| Compo- | | IL-18 | ka | kd | KD |
|---|---|---|---|---|---|
| sition | Antibody | polypeptide | (1/Ms) | (1/s) | (nM) |
| — | Pembrolizumab/ | — | 4.00E+05 | 2.72E−03 | 6.77 |
| | Keytruda | | | | |
| | LZM-009 | — | 3.42E+05 | 7.70E−03 | 23.50 |
| A | LZM-009 | SEQ ID NO: | 4.54E+05 | 4.79E−03 | 11.50 |
| | | 30 | | | |

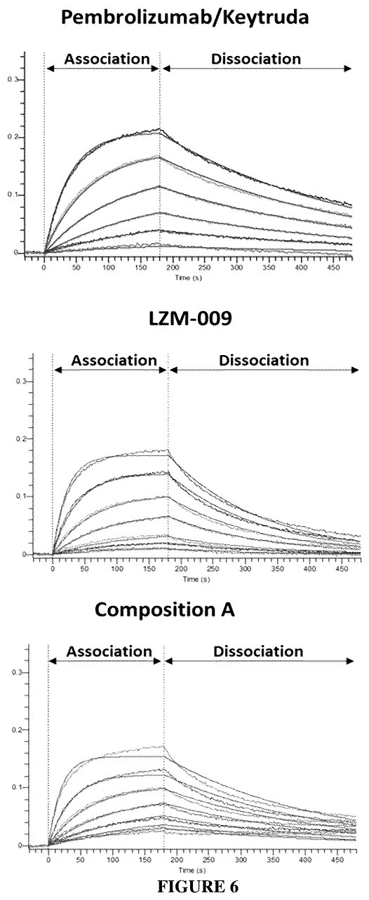

FIG. 6 shows plots measuring ability of the unmodified and of conjugated antibodies to bind to human PD-L1/B7-H1 ligand, with the figure showing net BioLayer interferometry shift in nanometer on the y-axis and time of incubation dosage of the biotinylated PD-L1 protein on the x-axis. The figure shows mean ELISA signal on the y-axis and dosage of the human Fc gamma receptors on the x-axis. The unconjugated reference antibodies are Pembrolizumab and LZM-009. The conjugated antibodies tested is Compositions A. It is predicted that IL-18 fusion immunocytokines will display similar results.

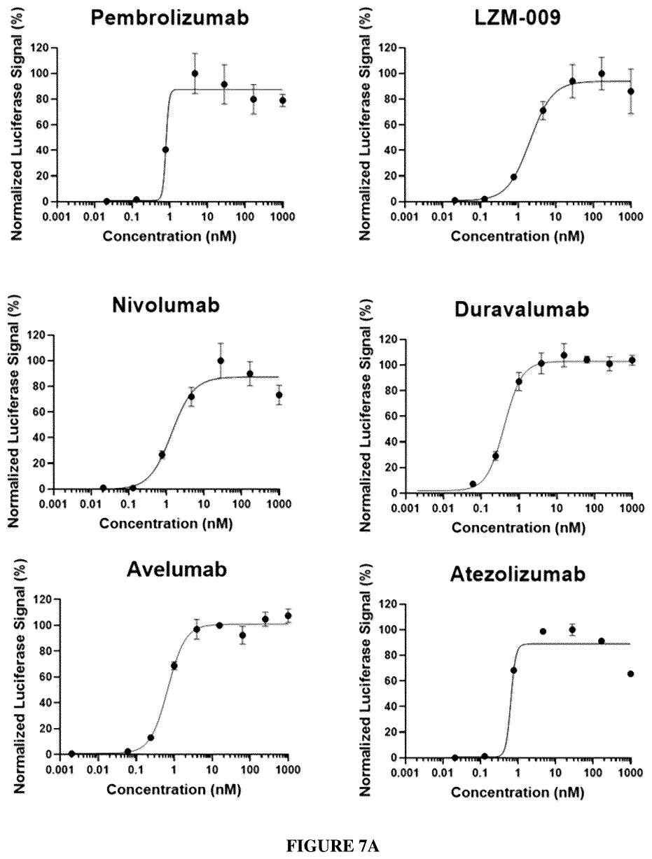

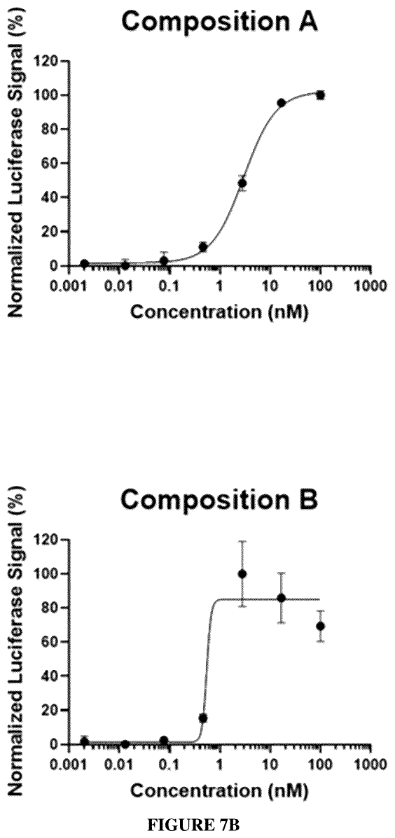

### Example 7-PD-1/PD-L1 Blockade Assay of Conjugated Immunocytokines (FIGS. 7A and 7B)

For immunocytokine compositions which comprise PD-1 or PD-L1 antibodies or antigen binding fragments, the experiment outlined below is performed to assess the ability of the immunocytokines to interfere with the PD-1/PD-L1 pathway. The assay is the PD-1/PD-L1 Blockade Bioassay from Promega (Cat #J1250, Madison, WI, USA). PD-1/PD-L1 Blockade Bioassay is a bioluminescent cell-based assay based on the co-culture of effector cells with target cells mimicking an immunological synapse. Jurkat T cells expressing human PD-1 and a luciferase reporter driven by a NFAT response element (NFAT-RE) are activated by CHO-K1 cells expressing human PD-L1 and an engineered cell surface protein designed to activate Jurkat cells cognate TCRs. Concurrent interaction PD-1/PD-L1 inhibits TCR signaling and represses NFAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture 96-wells plates and cultured overnight at 37° C./5% CO₂. Test molecules were measured in four-fold serial dilutions starting at 1 μM down to 0.002 nM and pre-incubated on target cells for 10 min before the addition of freshly thawed PD-1 Jurkat effector cells. After 6 h at 37° C./5% CO₂, activity NFAT-RE luminescent reporter was evaluated by the addition of Bio-Glo reagent and measured on an ENSPIRE® plate reader (1 sec/well) from Perkin Elmer (Schwerzenbach, Switzerland). Results from this experiment are shown in the table below.

Activity of unconjugated IL-18 variants and corresponding IL-18
immunocytokines in the PD-1/PD-L1 blockade cellular assay

| | | | KD FcRn |
|---|---|---|---|
| Composition | Antibody | IL-18 polypeptide | (nM) |
| — | Pembrolizumab/Keytruda | — | 0.757 |
| — | LZM-009 | — | 5.799 |
| — | Durvalumab/Imfinzi | — | 0.330 |
| — | Atezolizumab/Tecentriq | — | 0.664 |
| — | Avelumab/Bavencio | — | 0.676 |
| — | Trastuzumab/Herceptin | | >10000 |
| A | LZM-009 | SEQ ID NO: 30 | 4.075 |
| B | Atezolizumab/Tecentriq | SEQ ID NO: 30 | 0.573 |
| C | Trastuzumab/Herceptin | SEQ ID NO: 30 | NT |

FIGS. 7A and 7B shows plots measuring ability of the unmodified and of conjugated anti-PD1 antibodies to interfere with PD1/PDL1 pathway, with the figure showing normalized luminescence intensity of effector cells NFAT-Lucia reporter on the y-axis and dosage of the unmodified and of conjugated anti-PD1 antibodies on the x-axis. The unconjugated reference antibodies are Pembrolizumab, LZM-009, Nivolumab, Atezolizumab, Durvalumab, and Avelumab. The conjugated antibodies tested in this figure are compositions A and composition B. It is predicted that IL-18 fusion immunocytokines will display similar results.

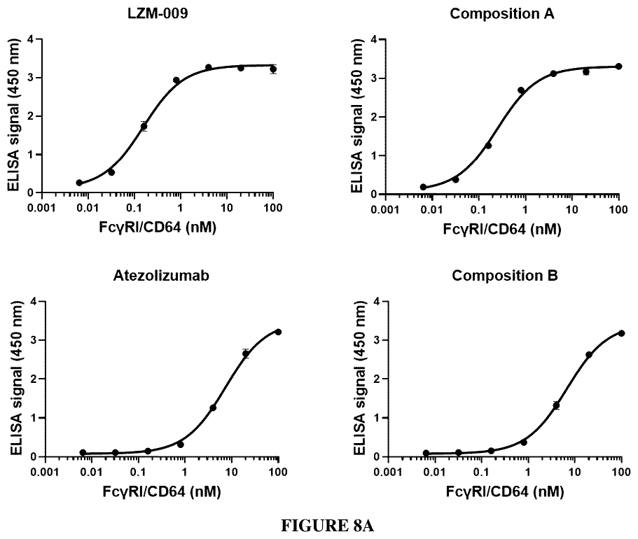

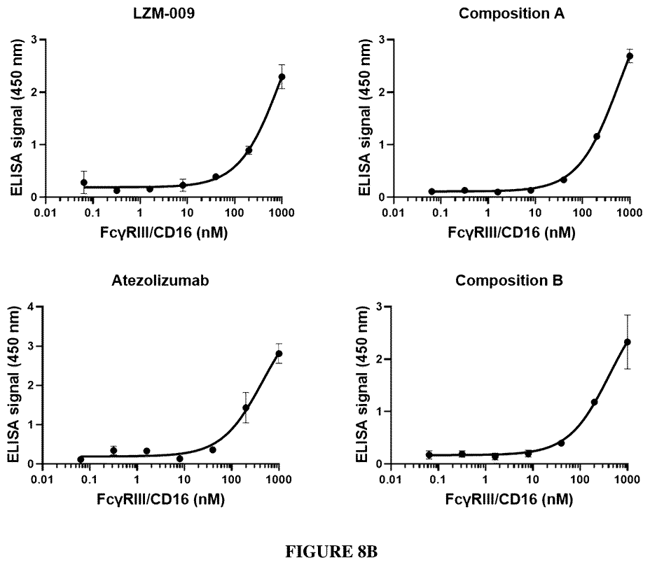

### Example 8-Human FcγR Binding Assay of Conjugated Immunocytokines (FIG. 8A and FIG. 8B)

The interaction of the unmodified and of conjugated antibodies with human Fc gamma receptors I (FcγRI/CD64), with human Fc gamma receptors IIa (FcγRIIa/CD32a), with inhibitory human Fc gamma receptors IIb (FcγRIIb/CD32b), and with human Fc gamma receptors III FcγRIIIa/CD16 were measured by ELISA.

Briefly, Corning high-binding half-area plates (Fisher Scientific, Reinach, Switzerland) were coated overnight at 4° C. with 25 ul of unmodified and of conjugated anti-PD1 antibodies at 2.5 μg/ml in PBS. Plates were then washed four times with 100 ul of PBS-0.02% Tween20. Plates surfaces were blocked with 25 ul of PBS-0.02% Tween20-1% BSA at 37° C. during 1h. Plates were then washed four times with 100 ul of PBS-0.02% Tween20. Then twenty-five microliters of either recombinant Human Fc gamma RI/CD64 Protein (R&D systems, 1257-FC-050, CF), recombinant Human Fc gamma RIIA/CD32a (H167) Protein (R&D systems, 9595-CD-050, CF), recombinant Human Fc gamma RIIB/CD32b Avi-tag Protein (R&D systems, AVI1875-050, CF), or recombinant Human Fc gamma RIIIA/CD16a Protein (R&D systems, 4325-FC-050; CF) were added in five-fold serial dilutions ranging from 1000 nM to 0.001 nM into PBS-0.02% Tween20-0.1% BSA and incubated at 37° C. during 2h. Plates were then washed four times with 100 ul of PBS-0.02% Tween20. Twenty-five microliters of a 1/500 HRP-anti-His antibody in PBS-0.02% Tween20-0.1% BSA (R&D systems, anti-HIS-HRP Ab, #MAB050H) were added to each well and plates were incubated at Room Temperature during 1h. Plates were then washed four times with 100 ul of PBS-0.02% Tween20. Fifty microliters of TMB substrate reagent (#C_{L}07, Merck, Buchs, Switzerland) were added to each well and incubated at 37° C. during 5 min. After 5 min at 37° C., Horseradish peroxidase reaction was stopped by adding 50 ul/well of 0.5M H2SO₄ stop solution. ELISA signal was then measured at 450 nm on an EnSpire plate reader from Perkin Elmer (Schwerzenbach, Switzerland). Results from this experiment are shown in the table below.

Binding affinity of reference antibodies and conjugated immunocytokines
with human Fc gamma Receptors as measured by ELISA

| | | | FcγRI/ | FcγRIIa | FcγRIIIa | FcγRIIb |
|---|---|---|---|---|---|---|
| | | IL-18 | (CD64) | (CD32a) | (CD16) | (CD32b) |
| Composition | Antibody | polypeptide | nM | nM | nM | nM |
| — | Pembrolizumab/Keytruda | — | 0.5932 | 3358 | 1660 | >10000 |
| | LZM-009 | — | 0.3150 | 1348 | 1707 | 2627 |
| — | Durvalumab/Imfinzi | — | 2.34 | 160 | 234 | 233 |
| — | Atezolizumab/Tecentriq | — | 9.17 | 1370 | 356 | >10000 |
| — | Trastuzumab/Herceptin | — | 0.0785 | 350 | 807 | 360 |
| A | LZM-009 | SEQ ID | 0.25 | 1892 | 598 | 436 |
| | | NO: 30 | | | | |
| B | Atezolizumab/Tecentriq | SEQ ID | 10.85 | 1740 | 492 | >10000 |
| | | NO: 30 | | | | |
| C | Trastuzumab/Herceptin | SEQ ID | | | 3299 | |
| | | NO: 30 | | | | |

NT: Not Tested

FIG. 8A and FIG. 8B show plots measuring ability of the unmodified and of conjugated antibodies to bind to human Fc gamma receptor I (CD64) on top panels, and to human Fc gamma receptor IIIa (CD16) on lower panels. The figure shows mean ELISA signal on the y-axis and dosage of the human Fc gamma receptors on the x-axis. The unconjugated reference antibodies are LZM-009 and Atezolizumab. The conjugated antibodies tested are Compositions A and B. It is predicted that IL-18 fusion immunocytokines will display similar results.

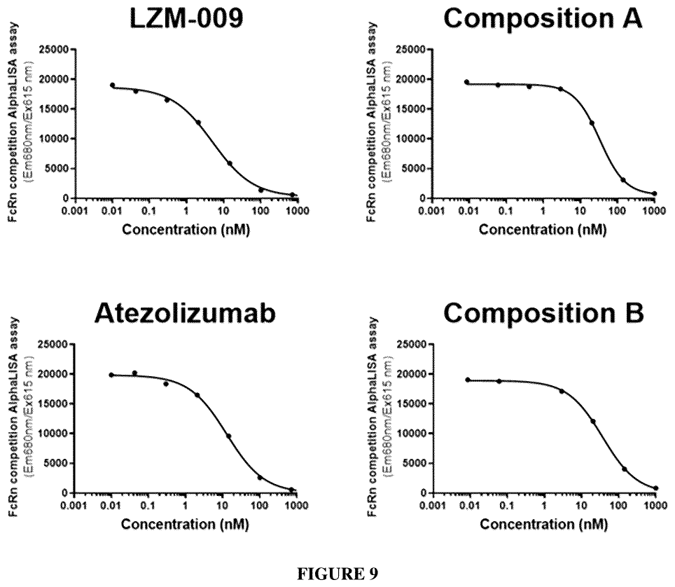

### Example 9-Human FcRn Binding Assay for Conjugated Immunocytokines (FIG. 9)

The interaction of the unmodified and of conjugated anti-PD1 antibodies with the human neonatal Fc receptor (FcRn) at pH 6 was measured using the AlphaLISA® Human FcRn Binding Kit (AL3095C) from Perkin Elmer (Schwerzenbach, Switzerland). The AlphaLISA® detection of FcRn and IgG binding uses IgG coated AlphaLISA® acceptor beads to interact with biotinylated human FcRn captured on Streptavidin-coated donor beads. When reference IgG binds to FcRn, donor and acceptor beads come into proximity enabling the transfer of singlet oxygen that trigger a cascade of energy transfer reactions in the acceptor beads, resulting in a sharp peak of light emission at 615 nm. Addition of a free IgG antibodies into the AlphaLISA® mixture creates a competition for the binding of FcRn to the reference antibody resulting in a loss of signal.

Briefly, test molecules were measured in serial dilutions starting at 5 μM down to 64 μM and incubated with AlphaLISA® reaction mixture consisting of 800 nM of recombinant biotinylated human FcRn, 40 μg/ml of human IgG conjugated Acceptor beads, and 40 μg/ml of Streptavidin coated Donor beads in pH 6 MES buffer. After 90 min at 23° C. in the dark, AlphaLISA® signal was measured on an EnSpire plate reader (Excitation at 680 nm, Emission at 615 nm) from Perkin Elmer (Schwerzenbach, Switzerland). Results from this experiment are shown in the table below.

Binding affinity of reference antibodies and immunocytokines
with the human neonatal Fc receptor as measured by AlphaLISA

| | | | KD FcRn |
|---|---|---|---|
| Composition | Antibody | IL-18 polypeptide | (nM) |
| — | Pembrolizumab/Keytruda | — | 7.45 |
| — | LZM-009 | — | 6.00 |
| — | Durvalumab/Imfinzi | — | 8.31 |
| — | Atezolizumab/Tecentriq | — | 6.40 |
| — | Avelumab/Bavencio | — | 5.36 |
| — | Trastuzumab/Herceptin | | 6.55 |
| A | LZM-009 | SEQ ID NO: 30 | 33.45 |
| B | Atezolizumab/Tecentriq | SEQ ID NO: 30 | 36.68 |
| C | Trastuzumab/Herceptin | SEQ ID NO: 30 | 25.12 |

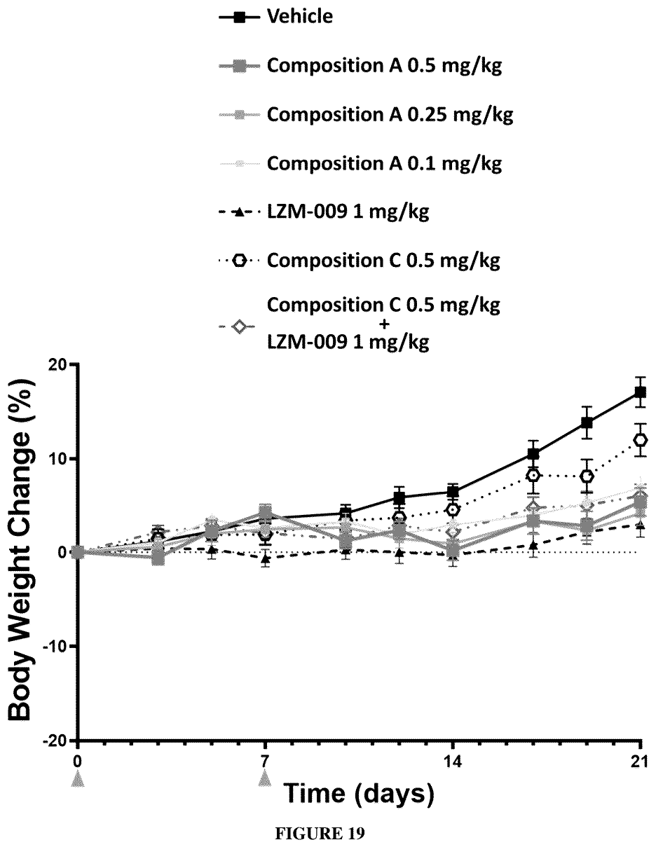

FIG. 19 shows plots measuring ability of the unmodified and of conjugated antibodies to bind to human Fc neonatal receptor. The figure shows mean AlphaLISA signal on the y-axis and dosage of the human Fc neonatal receptor (FcRn) on the x-axis. The unconjugated reference antibodies are LZM-009 and Atezolizumab. The conjugated antibodies tested are Compositions A and B. It is predicted that IL-18 fusion immunocytokines will display similar results.

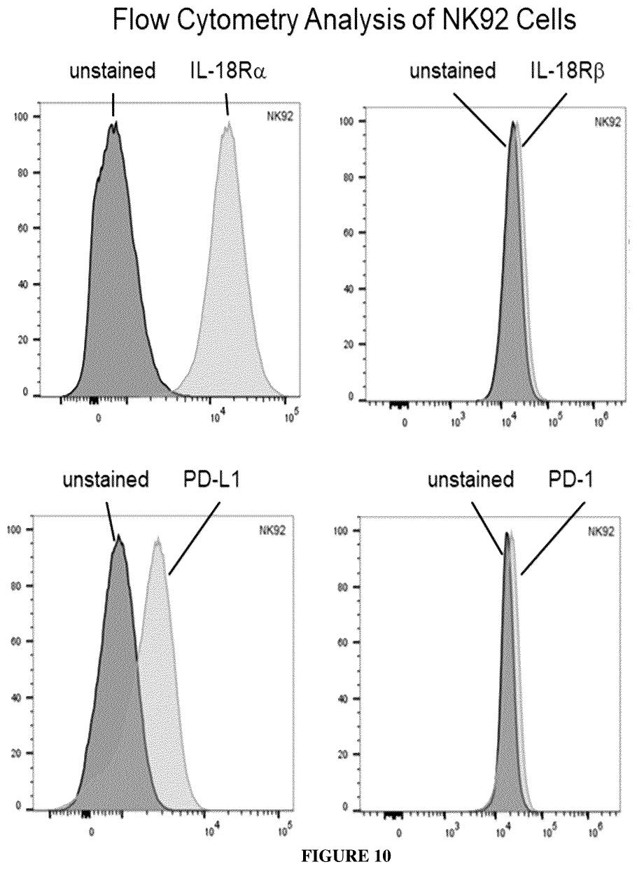

### Example 10-IFN gamma secretion assay in NK92 cells (FIGS. 10 & 11)

The IFNγ-secretion stimulating activity of the unconjugated IL-18 variants and corresponding IL-18 immunocytokines was evaluated on NK92 cell line. The NK cell line NK-92 derived from a patient with lymphoma (ATCC, Cat #CRL-2407) was cultured in aMEM medium supplemented with 12.5% FCS, 12.5% horse serum (HS), 50 μM B-mercaptoEthanol, and 2 ng/ml of recombinant Human Interleukin-2 (IL-2).

On the day of experiment, cells were harvested and washed with aMEM medium without IL-2 and resuspended in medium (w/o IL-2) containing 1 ng/ml of recombinant human Interleukin-12 (SinoBiologicals, Cat #CT011-H08H). After counting, cells were seeded at 100 000 cells/well in a 384 well titer plate and incubated at 37° C./5% CO2. Sixteen 4-fold serial dilutions of IL-18 analytes were prepared in aMEM medium-1 ng/ml IL-12 and were added to the NK-92 cells. Final IL-18 analytes concentration ranged from to 200 nM down to 0.01 μM.

After 16-20h incubation at 37° C./5% CO2, 5 μl of supernatant were carefully transferred to a 384 microwells OPTIPlate (Perkin Elmer; Cat #6007270) and Interferon-gamma (IFNY) levels measured using the Human IFNγ AlphaLISA Assay Kit (Perkin Elmer, Cat #AL217C). Briefly, 10 ul of 2.5X AlphaLISA Anti-IFNγ acceptor beads and biotinylated Antibody Anti-IFNγ mix were added to the 5 ul of NK-92 supernatants and incubated for 1 h at room temperature under shaking. Under subdued light, 2.5 ul of 2X streptavidin (SA) donor beads were pipetted in each well and incubated for 30 min at room temperature under shaking. AlphaLisa signal was then measured on an Enspire plate reader (Perkin Elmer) using 680 and 615 nm as excitation and emission wavelengths respectively. Half maximal effective concentration (EC₅₀) was calculated based on a variable slope, four parameter analysis using GraphPad PRISM software. Results from this experiment are shown in the table below.

Activity of unconjugated IL-18 variants and corresponding conjugated
IL-18 immunocytokines in the IFNγ secretion NK92 assay

| | | | EC₅₀ |
|---|---|---|---|
| Composition | Antibody | IL-18 polypeptide | (nM) |
| — | — | SEQ ID NO: 1 | 0.374 |
| — | — | SEQ ID NO: 30 | 0.002 |
| — | — | SEQ ID NO: 31 | 0.103 |
| | | SEQ ID NO: 60 | 0.008 |
| A | LZM-009 | SEQ ID NO: 30 | 0.073 |
| B | Atezolizumab/Tecentriq | SEQ ID NO: 30 | 0.001 |
| C | Trastuzumab/Herceptin | SEQ ID NO: 30 | 0.027 |
| D | LZM-009 | SEQ ID NO: 31 | 0.471 |
| E | LZM-009 | SEQ ID NO: 60 | 0.056 |
| F | Trastuzumab/Herceptin | SEQ ID NO: 60 | 0.064 |

FIG. 10 shows plots measuring the levels PD-1 and PD-L1 surface expression on NK92 cells.

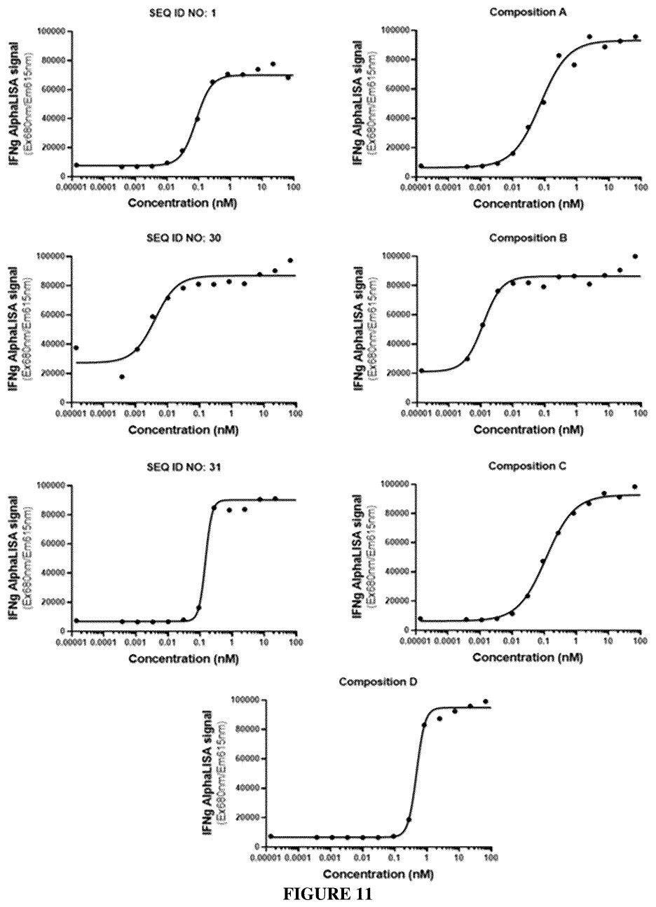

FIG. 11 shows plots measuring ability of the unconjugated IL-18 variants and corresponding IL-18 immunocytokines to stimulate the secretion of IFNgamma by NK92 cells. The figure shows mean IFNg alphaLISA signal on the y-axis and dosage of the unconjugated IL-18 variants and corresponding IL-18 immunocytokines on the x-axis. The unconjugated IL-18 variants are native IL-18 wild-type (SEQ ID N°: 1), SEQ ID N°: 30, and SEQ ID N°: 31.Corresponding IL-18 immunocytokines tested are Compositions A, B, C, and D. It is predicted that IL-18 fusion immunocytokines will display similar results.

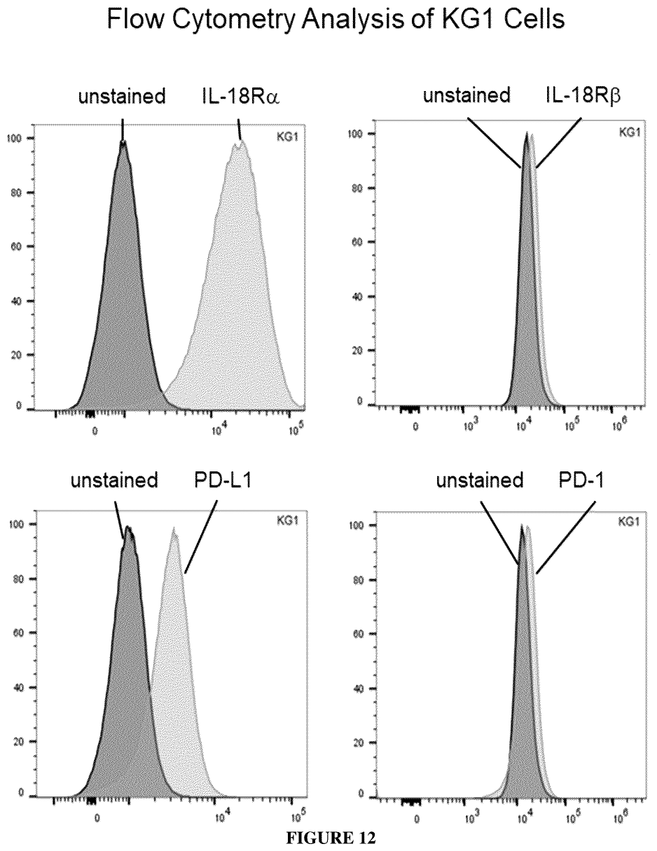

### Example 11-IFN gamma secretion assay in KG-1 cells for conjugated immunocytokines (FIG. 12-14)

The IL-18 responsive AML cell line KG-1 shows high expression of IL-18Ra and moderate levels of IL18RB, respectively. The KG-1 cell line was used to generate a PD-1 expressing cell line and furthermore, to measure IFNγ release upon incubation with IL-18 variants and corresponding IL-18 immunocytokines.

PD-1 expressing cell line generation: Briefly, KG-1 cells were transduced using lentiviral particles carrying the human PD-1 gene (PDCD1 NM_005018; Origene, CAT #:RC210364L3V) at a MOI (Multiplicity of Infection) of 30. Spinfection was performed at 1260g during 90 min at 37° C. in the presence of 5 μg/ml of Polybrene and 10 mM of HEPES in complete culture media (RPMI, 10% FBS, 1% L-Glutamine). Five days after transduction, puromycin at a final concentration of 1 μg/ml was added to select for PD-1 positive cells. For culture maintenance, puromycin concentration was decreased to 0.5 μg/ml. Stable and homogenous expression of PD-1 was verified by surface staining (BD Pharmingen, #557860).

IFNγ release was assessed in PD-1 positive (transduced) KG-1 cells, as well as in the parental PD-1^{negative} cells. 0.5×10⁵ cells were seeded into a 96-well U-bottom plate in culture media (RPMI, 10% FBS, 1% L-Glutamine) and stimulated with IL-18 variants/ICs for 20-24h. The test items were diluted to 100 nM in culture medium, followed by 7 10-fold serial dilutions. The lowest concentration assessed was 0.05 fM. After incubation, IFNγ release was measured using the LEGENDplex custom human mix and match KIT (Biolegend LEGENDplex™ Human IFN-γ Capture Bead B5, 13X #740942, LEGENDplex™ HU Essential Immune Response Panel Detection Abs, #740931, LEGENDplex™ Buffer Set A #740368). To this end, cell culture supernatant was collected and diluted 1:1 with Assay Buffer. Fluorescence measurements were done with a Quanteon Flow Cytometer from Acea Biosciences. For analysis, MFI values (median fluorescence intensity) were exported and plotted against concentrations used. The EC₅₀ values (half maximal effective concentration) were calculated based on a variable slope and four parameter analysis using GraphPad PRISM software version 9. Results from this experiment are shown in the table below. It is predicted that IL-18 fusion immunocytokines will display similar results.

Stimulation of IFNγ secretion by unconjugated
IL-18 variants and corresponding conjugated IL-18
immunocytokines in parental and PD-1 transduced KG-1 cells

| | | | | PD-1 | |
|---|---|---|---|---|---|
| | | | Parental | positive | PD-1^{pos}/ |
| | | IL-18 | KG-1 | KG-1 | PD-1^{neg} |
| | | poly- | EC₅₀ | EC₅₀ | EC₅₀ |
| Composition | Antibody | peptide | (nM) | (nM) | ratio |
| — | — | SEQ ID | 0.387 | 0.297 | 1.298 |
| | | NO: 1 | | | |
| — | — | SEQ ID | 0.012 | 0.008 | 1.336 |
| | | NO: 30 | | | |
| — | — | SEQ ID | 0.1485 | 0.1354 | 1.2 |
| | | NO: 31 | | | |
| A | LZM-009 | SEQ ID | 0.0110 | 0.0012 | 10.9 |
| | | NO: 30 | | | |
| B | Atezolizumab/ | SEQ ID | 0.0016 | 0.0036 | 0.6 |
| | Tecentriq | NO: 30 | | | |
| C | Trastuzumab/ | SEQ ID | 0.0166 | 0.0096 | 1.7 |
| | Herceptin | NO: 30 | | | |
| D | LZM-009 | SEQ ID | 0.2033 | 0.0225 | 9.3 |
| | | NO: 31 | | | |

FIG. 12 shows plots measuring the levels PD-1 and PD-L1 surface expression on KG-1 cells.

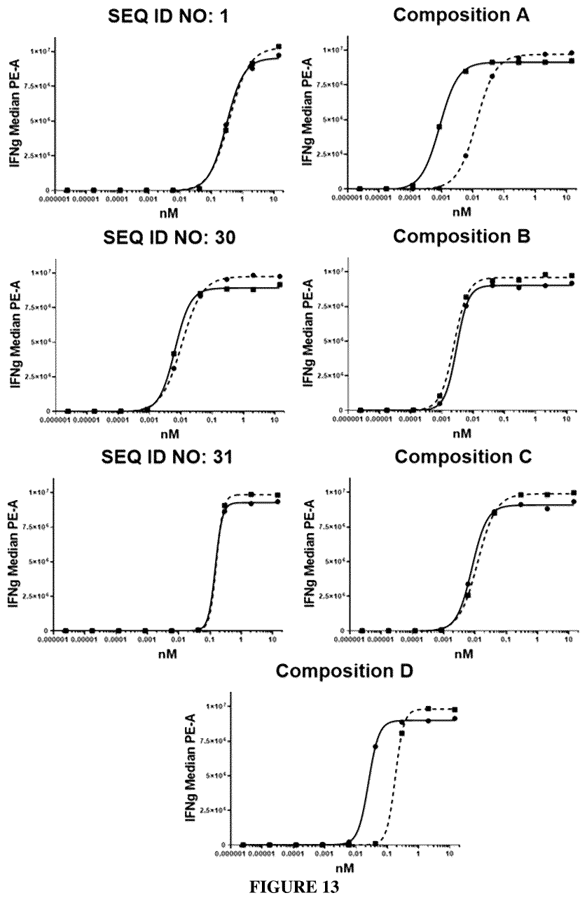

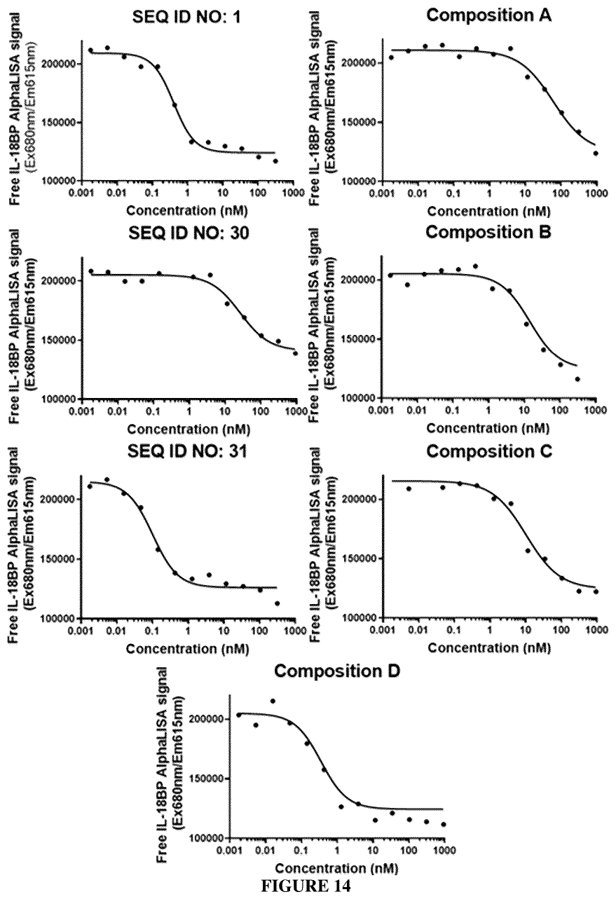

FIG. 13 shows plots measuring the ability of the unconjugated IL-18 variants and corresponding IL-18 immunocytokines to stimulate the secretion of IFNgamma by parental PD-1^{negative} and by engineered PD-1^{positive} KG-1 cells. The figure shows mean IFNg legendplex signal on the y-axis and dosage of the unconjugated IL-18 variants and corresponding IL-18 immunocytokines on the x-axis. The unconjugated IL-18 variants are native IL-18 wild-type (SEQ ID N°: 1), SEQ ID N°: 30, and SEQ ID N°: 31. Corresponding IL-18 immunocytokines tested are Compositions A, B, C, and D. It is predicted that IL-18 fusion immunocytokines will display similar results.

### Example 12-IL-18 Binding Protein alphaLISA Assay for Conjugated Immunocytokines (FIG. 14)

Wild type or IL-18 polypeptides samples were diluted at 5.6 μM in a solution of 1× alphaLISA Immunoassay Buffer provided in the alphaLISA IFNγ Detection kit and were diluted applying 3-fold serial dilutions down to 1.7 μM in 384 deep well plates. A solution of 10 ng/ml of human IL-18BP-His was prepared with 1× alphaLISA Immunoassay Buffer. IL-18/IL-18BP complex formation was performed incubating 30 μl of IL-18BP solution to IL-18 sample titrations for 1 h at 20° C. IL-18BP standard was prediluted from stock solution supplied in alphaLISA IFNγ Detection kit at 100 ng/ml with 1× alphaLISA Buffer and titration prepared from applying 2-fold serial dilutions. The following solutions were prepared: a 50 μg/ml solution of anti-IL-18BP alphaLISA Acceptor beads, a 5 nM solution of biotinylated anti-IL18BP antibody and a 80 μg/ml light-protected solution of Streptavidin Donor beads in 1× alphaLISA Immunoassay Buffer. To detect unbound IL-18BP in IL-18/IL-18P complex samples, 5 μl of pre-mixed Acceptor beads solution were transferred on top on 7.5 μL of samples in 384-well Optiplates, followed by a short centrifugation step at 150g, and incubated for 30 minutes at 20° C. under shaking at 750 rpm.

5 μl of Biotinylated anti-IL-18BP antibody were added, followed by a short centrifugation step at 150g, and incubated for 60 minutes at 20° C. under shaking at 750 rpm. Under subdued light, 12.5 μl of pre-mixed Donor beads were added, followed by a short centrifugation step at 150g, and incubated for 30 minutes at 20° C. under shaking at 750 rpm with no light. AlphaLisa signal was then measured on an Enspire plate reader (Perkin Elmer) using 680 and 615 nm as excitation and emission wavelengths respectively. Unbound IL-18BP concentration interpolated from the standard signal-concentration curve using GraphPad Prism. Results from this experiment are shown in the table below. It is predicted that IL-18 fusion immunocytokines will display similar results.

Binding affinity of reference antibodies and
conjugated immunocytokines with the human IL-18
Binding Protein as measured by AlphaLISA

| | | | IL-18BP |
|---|---|---|---|
| | | IL-18 | KD |
| Composition | Antibody | polypeptide | (nM) |
| — | — | SEQ ID NO: 1 | 0.411 |
| — | — | SEQ ID NO: 30 | 24.600 |
| — | — | SEQ ID NO: 31 | 0.103 |
| A | LZM-009 | SEQ ID NO: 30 | 10.930 |
| B | Atezolizumab/Tecentriq | SEQ ID NO: 30 | 6.382 |
| C | Trastuzumab/Herceptin | SEQ ID NO: 30 | 10.496 |
| D | LZM-009 | SEQ ID NO: 31 | 15.263 |

FIG. 14 shows plots measuring the ability of the unconjugated IL-18 variants and corresponding IL-18 immunocytokines to bind to the human IL-18 Binding Protein (IL-18BP). The figure shows mean free IL-18BP AlphaLISA signal on the y-axis and dosage of the unconjugated IL-18 variants and corresponding IL-18 immunocytokines on the x-axis. The unconjugated IL-18 variants are native IL-18 wild-type (SEQ ID N°: 1), SEQ ID N°: 30, and SEQ ID N°: 31. Corresponding IL-18 immunocytokines tested are Compositions A, B, C, and D. It is predicted that IL-18 fusion immunocytokines will display similar results.

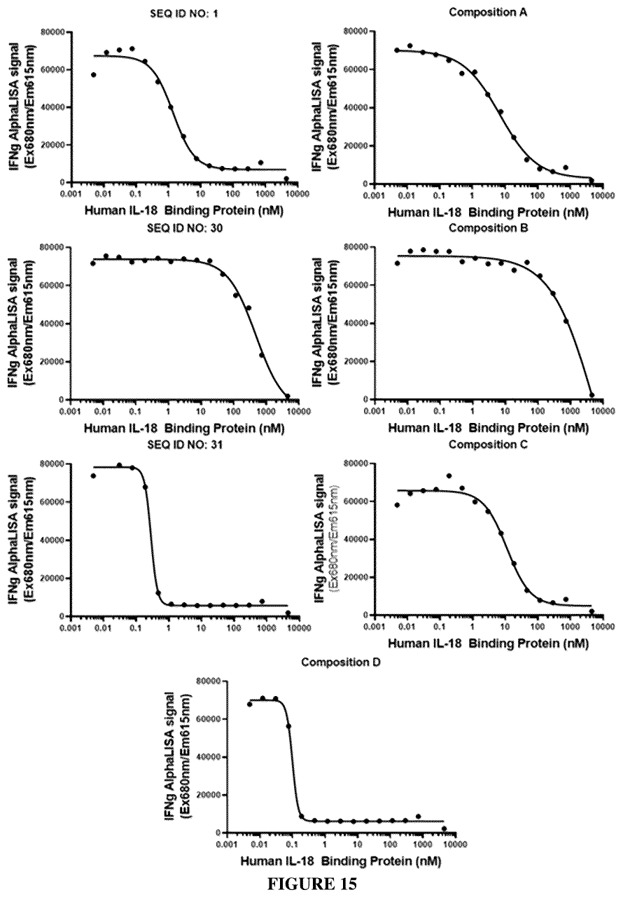

### Example 13-Cellular IL-18 Binding Protein Resistance Assay for Conjugated Immunocytokines (FIG. 15)

The NK cell line NK-92 derived from a patient with lymphoma (ATCC, Cat #CRL-2407) was cultured in aMEM medium supplemented with 20% FCS-Glutamax, 25 μM B-mercaptoEthanol, and 100 IU/ml of recombinant Human Interleukin-2 (IL-2). On the day of experiment, cells were harvested and washed with aMEM medium without IL-2. After counting, cells were seeded at 100 000 cells/well in a 384 well titer plate and incubated at 37° C./5% CO2. Sixteen 2-fold serial dilutions of Fc fused human IL-18 binding protein isoform a (IL-18BPa; R&D systems, Cat #119-BP) were prepared in aMEM medium-1 ng/ml IL-12 containing 2 nM of each IL-18 variants and were added to the NK-92 cells. Final IL-18 analytes concentration was 1 nM and final IL-18BPa concentrations ranged from to 566 nM down to 17 μM.

After 16-20h incubation at 37° C./5% CO2, 5 μl of supernatant were carefully transferred to a 384 microwells OPTIplate (Perkin Elmer; Cat #6007270) and Interferon-gamma (IFNY) levels measured using the Human IFNγ AlphaLISA Assay Kit (Perkin Elmer, Cat #AL217C). Briefly, 10 μl of 2.5X AlphaLISA Anti-IFNγ acceptor beads and biotinylated Antibody Anti-IFNγ mix were added to the 5 μl of NK-92 supernatants and incubated for 1 h at room temperature under shaking. Under subdued light, 2.5 μl of 2X streptavidin (SA) donor beads were pipetted in each well and incubated for 30 min at room temperature under shaking. AlphaLisa signal was then measured on an Enspire plate reader (Perkin Elmer) using 680 and 615 nm as excitation and emission wavelengths respectively. Half maximal inhibitory concentration (IC50) was calculated based on a variable slope, four parameter analysis using GraphPad PRISM software. Results from this experiment are shown in the table below. It is predicted that IL-18 fusion immunocytokines will display similar results.

IL-18BP-mediated inhibition of IFNγ secretion
by NK92 cells stimulated with unconjugated IL-18
variants and corresponding IL-18 immunocytokines

| | | IL-18 | IC₅₀ |
|---|---|---|---|
| Composition | Antibody | polypeptide | (nM) |
| — | — | SEQ ID NO: 1 | 0.781 |
| — | — | SEQ ID NO: 30 | 487.8 |
| — | — | SEQ ID NO: 31 | 0.282 |
| A | LZM-009 | SEQ ID NO: 30 | 6.87 |
| B | Atezolizumab/Tecentriq | SEQ ID NO: 30 | >1000 |
| C | Trastuzumab/Herceptin | SEQ ID NO: 30 | 21.44 |
| D | LZM-009 | SEQ ID NO: 31 | 0.098 |

FIG. 15 shows plots measuring the ability of the human IL-18 Binding Protein to inhibit the secretion of IFNgamma by NK92 cells stimulated with 2 nM of unconjugated IL-18 variants and corresponding IL-18 immunocytokines. The figure shows mean IFNg alphaLISA signal on the y-axis and dosage of the human IL-18 Binding Protein on the x-axis. The unconjugated IL-18 variants are native IL-18 wild-type (SEQ ID N°: 1), SEQ ID N°: 30, and SEQ ID N°: 31. Corresponding IL-18 immunocytokines tested are Compositions A, B, C, and D. It is predicted that IL-18 fusion immunocytokines will display similar results.

### Example 14-In Vivo Antitumor Activity in MC38 Colon Carcinoma Model (Conjugated Immunocytokines) (FIG. 16-18)

An in vivo efficacy study was performed in mice. Naïve, 6-8 weeks old, C57BL/6-hPD1 female mice (GemPharmatech Co, Ltd, Nanjing, China) were inoculated subcutaneously at the right upper flank with MC38 tumor cells (3×10⁵) in 0.1 mL of PBS for tumor development. The animals were randomized (using an Excel-based randomization software performing stratified randomization based upon tumor volumes), and treatment started when the average tumor volume reached approximately 120 mm³. Animals treated with unmodified antibodies received two weekly 10 mL/kg bolus intraperitoneal (i.p.) injections. Animals treated with IL-18 polypeptide conjugated antibodies received two weekly 10 mL/kg bolus intravenous (i.v.) injections. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

FIG. 16A shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows mean tumor volume on the y-axis and time on the x-axis. The immunocytokine tested in this figure is Composition A tested as a single agent at 0.3 and 1 mg/kg as two weekly i.v. injections. (n=9; mean±SEM). It is predicted that IL-18 fusion immunocytokines will display similar results.

FIG. 16B shows a plot describing the effect of unmodified PD-L1 antibodies and of IL-18 polypeptide conjugated PD-L1 antibody on the growth of MC38 syngeneic colon carcinoma tumors in hPD1 C57BL/6 mice. The figure shows mean tumor volume on the y-axis and time on the x-axis. The immunocytokine tested in this figure is Composition B tested as a single agent at 1 and 3 mg/kg as two weekly i.v. injections. (n=9; mean±SEM). It is predicted that IL-18 fusion immunocytokines will display similar results.

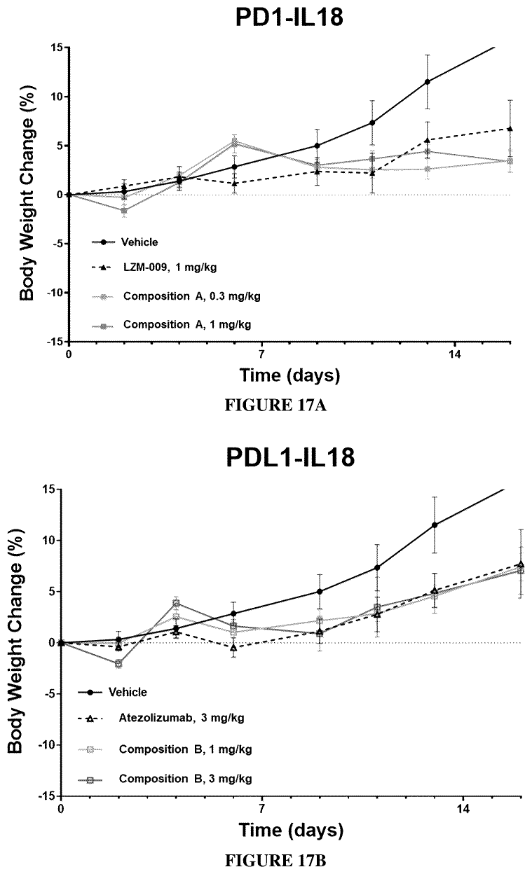

FIG. 17A shows a plot describing the effect of unmodified PD-1 antibodies and of IL-18 polypeptide conjugated PD-1 antibody on the body weight of MC38 syngeneic colon carcinoma tumor-bearing hPD1 C57BL/6 mice. The figure shows mean body weight change on the y-axis and time on the x-axis. The immunocytokine tested in this figure is Composition A tested as a single agent at 0.3 and 1 mg/kg as two weekly i.v. injections. (n=9; mean±SEM). It is predicted that IL-18 fusion immunocytokines will display similar results.

FIG. 17B shows a plot describing the effect of unmodified PD-L1 antibodies and of IL-18 polypeptide conjugated PD-L1 antibody on the body weight of MC38 syngeneic colon carcinoma tumor-bearing hPD1 C57BL/6 mice. The figure shows mean body weight change on the y-axis and time on the x-axis. The immunocytokine tested in this figure is Composition B tested as a single agent at 1 and 3 mg/kg as two weekly i.v. injections. (n=9;mean±SEM). It is predicted that IL-18 fusion immunocytokines will display similar results.

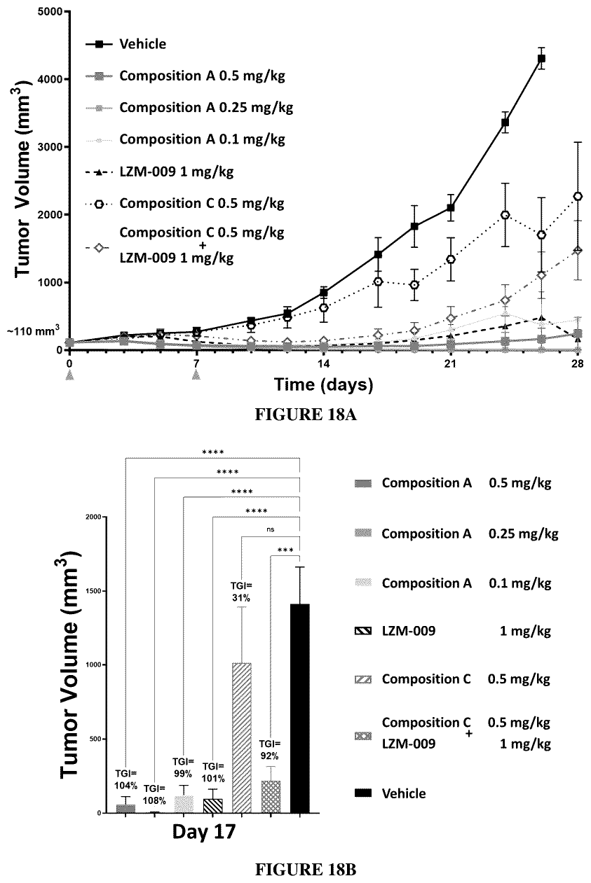

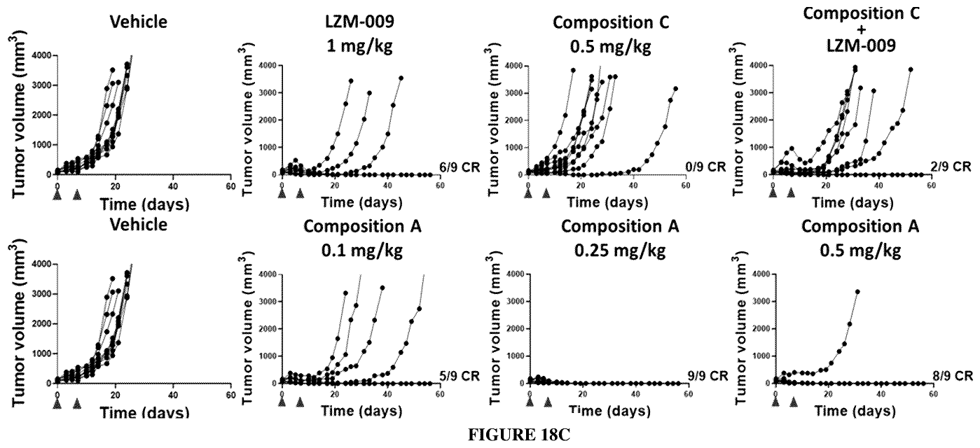

### Example 15-In Vivo Antitumor Activity in MC38 Colon Carcinoma Model for Conjugated Immunocytokines (FIG. 18-21)

An in vivo efficacy study was performed in mice. Naïve, 6-8 weeks old, C57BL/6-hPD1 female mice (GemPharmatech Co, Ltd, Nanjing, China) were inoculated subcutaneously at the right upper flank with MC38 tumor cells (3×10⁵) in 0.1 mL of PBS for tumor development. The animals were randomized (using an Excel-based randomization software performing stratified randomization based upon tumor volumes), and treatment started when the average tumor volume reached approximately 110 mm³. Animals treated with unmodified antibodies received two weekly 10 mL/kg bolus intraperitoneal (i.p.) injections. Animals treated with IL-18 polypeptide conjugated antibodies received two weekly 10 mL/kg bolus intravenous (i.v.) injections. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a x b² where a and b are the long and short diameters of the tumor, respectively. Death and observed clinical signs were recorded on the basis of the numbers of animals within each subset.

FIG. 18A shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows mean tumor volume on the y-axis and time on the x-axis. The immunocytokine tested in this figure are composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM). It is predicted that IL-18 fusion immunocytokines will display similar results.

FIG. 18B shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows the mean tumor volume on day 17 post treatment initiation on the y-axis. The immunocytokine tested in this figure are composition A tested as a single agent at 0.1, 0.25, and 0.5 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; One-way Anova test *** P-value<0.001, ** P-value<0.01, * P-value<0.1, ns not significant, TGI: Tumor Growth Inhibition). It is predicted that IL-18 fusion immunocytokines will display similar results.

FIG. 18C shows a plot describing the effect of unmodified PD-1 antibodies and of IL-18 polypeptide conjugated PD-1 antibody on the growth of MC38 syngeneic colon carcinoma tumors in hPD1 C57BL/6 mice. The figure shows the tumor volume of each individual animal on the y-axis and time on the x-axis. The immunocytokine tested in this figure are composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; CR: Complete Response). It is predicted that IL-18 fusion immunocytokines will display similar results.

FIG. 19 shows a plot describing the effect of unmodified PD-L1 antibodies and of IL-18 polypeptide conjugated PD-1 antibody on the on the body weight of MC38 syngeneic colon carcinoma tumor-bearing hPD1 C57BL/6 mice. The figure shows mean body weight change on the y-axis and time on the x-axis. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM). It is predicted that IL-18 fusion immunocytokines will display similar results.

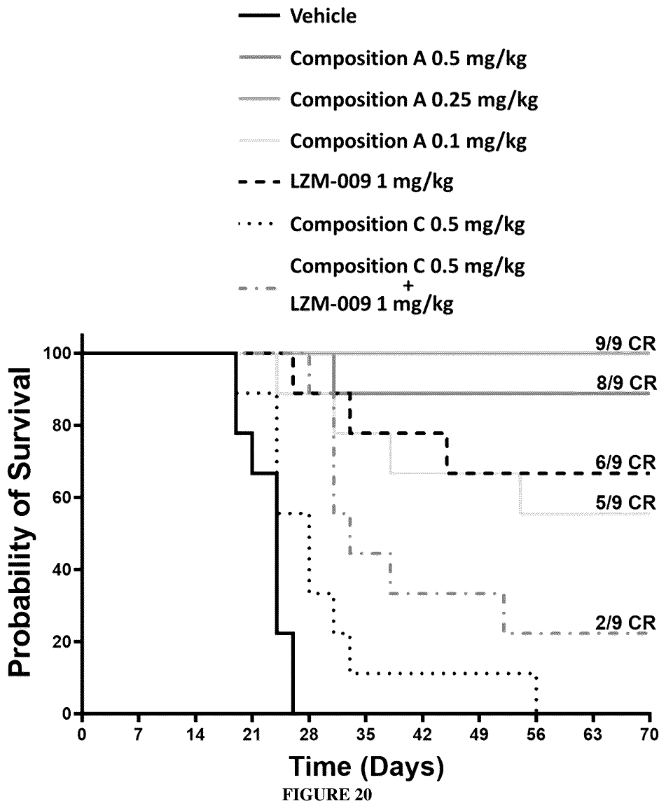

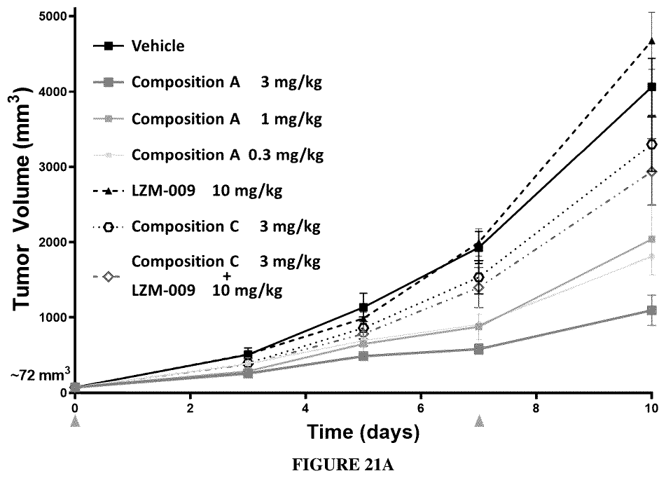

FIG. 20 shows a plot describing the effect of unmodified PD-L1 antibodies and of IL-18 polypeptide conjugated PD-1 antibody on the survival of MC38 syngeneic colon carcinoma tumor-bearing hPD1 C57BL/6 mice. The immunocytokine tested in this figure are Composition A tested as a single agent at 0.1, 0.25, and 0.5 mg/kg as two weekly i.v. injections. As a control, Her2-targeted immunocytokine composition C was applied at 0.5 mg/kg as a single agent and in combination with LZM-009 anti-PD-1 antibody at 1 mg/kg (n=9; mean±SEM; CR: Complete response). It is predicted that IL-18 fusion immunocytokines will display similar results.

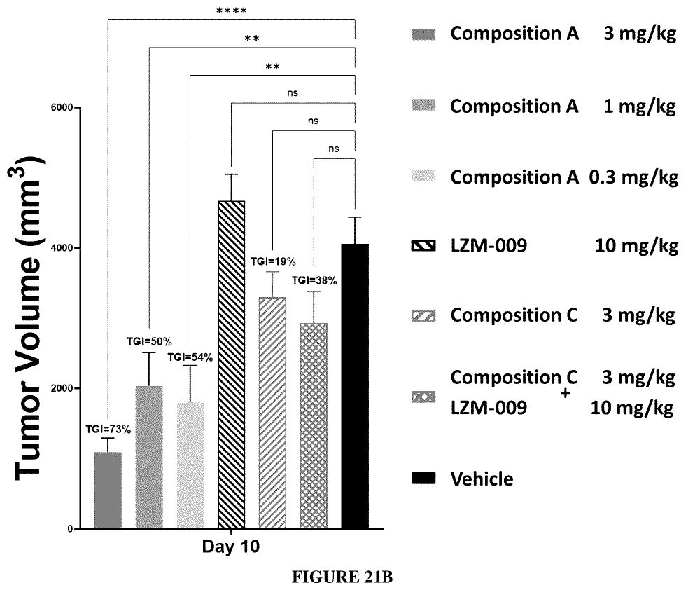

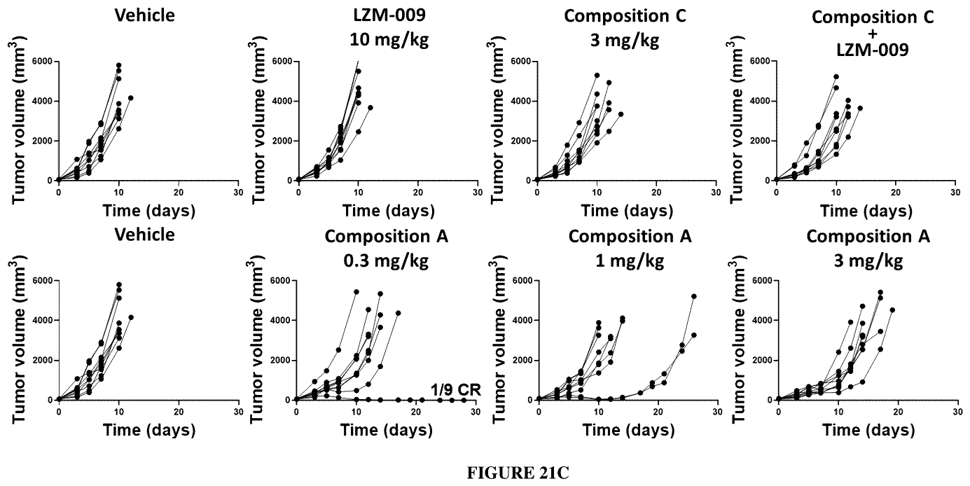

### Example 16-In Vivo Antitumor Activity in B16F10 Melanoma Model for Conjugated Immunocytokines (FIG. 21-24)

An in vivo efficacy study was performed in mice. Naïve, 6-8 weeks old, C57BL/6-hPD1 female mice (GemPharmatech Co, Ltd, Nanjing, China) were inoculated subcutaneously at the right upper flank with B16F10 tumor cells (5×10⁴; 1:1 with Matrigel®) in 0.1 mL of PBS for tumor development. The animals were randomized (using an Excel-based randomization software performing stratified randomization based upon tumor volumes), and treatment started when the average tumor volume reached approximately 70 mm³. Animals treated with unmodified antibodies received two weekly 10 mL/kg bolus intraperitoneal (i.p.) injections. Animals treated with IL-18 polypeptide conjugated antibodies received two weekly 10 mL/kg bolus intravenous (i.v.) injections. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a x b² where a and b are the long and short diameters of the tumor, respectively. Death and observed clinical signs were recorded on the basis of the numbers of animals within each subset.

FIG. 21A shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows mean tumor volume on the y-axis and time on the x-axis. The immunocytokine tested in this figure are composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM). It is predicted that IL-18 fusion immunocytokines will display similar results.

FIG. 21B shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows the mean tumor volume on day 10 post treatment initiation on the y-axis. The immunocytokine tested in this figure are composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; TGI: Tumor Growth Inhibition). It is predicted that IL-18 fusion immunocytokines will display similar results.

FIG. 21C shows a plot describing the effect of unmodified PD-1 antibodies and of IL-18 polypeptide conjugated PD-1 antibody on the growth of B16F10 syngeneic melanoma tumors in hPD1 C57BL/6 mice. The figure shows the tumor volume of each individual animal on the y-axis and time on the x-axis. The immunocytokine tested in this figure are composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; CR: Complete Response). It is predicted that IL-18 fusion immunocytokines will display similar results.

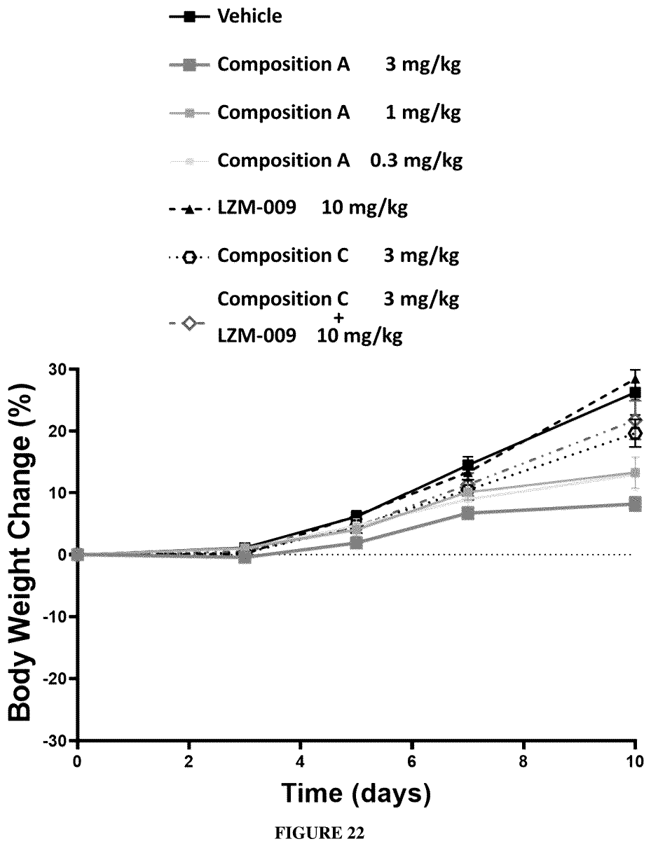

FIG. 22 shows a plot describing the effect of unmodified PD-L1 antibodies and of IL-18 polypeptide conjugated PD-1 antibody on the body weight of B16F10 syngeneic melanoma tumor-bearing hPD1 C57BL/6 mice. The figure shows mean body weight change on the y-axis and time on the x-axis. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM). It is predicted that IL-18 fusion immunocytokines will display similar results.

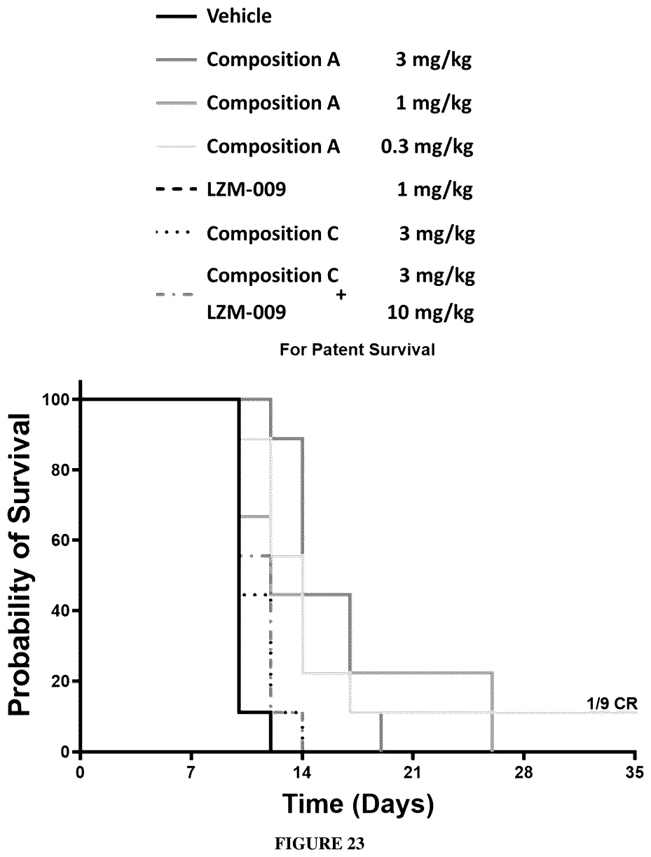

FIG. 23 shows a plot describing the effect of unmodified PD-L1 antibodies and of IL-18 polypeptide conjugated PD-1 antibody on the survival of B16F10 syngeneic melanoma tumor-bearing hPD1 C57BL/6 mice. The immunocytokine tested in this figure are Composition A tested as a single agent at 0.3, 1, and 3 mg/kg as two weekly i.v. injections. As a control, Her2-targeted immunocytokine composition C was applied at 3 mg/kg as a single agent and in combination with LZM-009 anti-PD-1 antibody at 10 mg/kg (n=9; mean±SEM; CR: Complete response). It is predicted that IL-18 fusion immunocytokines will display similar results.

### Example 17: Characteristics of Additional IL-18 Polypeptides

The following examples described additional IL-18 polypeptides which could be incorporated into immunocytokines as described herein (e.g., into fusion immunocytokines). It is predicted that these additional IL-18 polypeptides will behave analogously to those explicitly made herein.

17A-HEK-Blue Reporter Assay-An IL-18R positive HEK-Blue reporter cell line is used to determine binding of IL-18 variants to IL-18R and subsequent downstream signaling. The general protocol is outlined below.

5×10⁴ cells HEK-Blue IL18R reporter cells (InvivoGen, #hkb-hmil18) are seeded into each well of a 96 well plate and stimulated with 0-100 nM of IL-18 polypeptide variants at 37° C. and 5% CO2. After 20h incubation, 20 μL of cell culture supernatant is then taken from each well and mixed with 180 μL QUANTI-Blue media in a 96 well plate, incubated for 1 hour at 37° C. and 5% CO2. The absorbance signal at 620 nm is then measured on an Enspire plate reader with 680 and 615 nm as excitation and emission wavelengths, respectively. Half Maximal Effective dose (EC₅₀) is calculated based on a variable slope, four parameter analysis using GraphPad PRISM software.

The HEK-Blue IL-18R reporter assay described above was performed on additional IL-18 polypeptides which can be incorporated into immunocytokine compositions provided herein. It is expected that the IL-18 polypeptides provided below would behave similarly to C086 (SEQ ID NO: 30) when incorporated into an immunocytokine composition (i.e., a conjugate immunocytokine or fusion immunocytokine) as those otherwise provided herein (e.g., the relative activity of the IL-18 polypeptide in the immunocytokine will be the same).

| SEQ ID NO: or | | EC₅₀ |
|---|---|---|
| Composition ID | Sequence modifications | (pM) |
| 1 | Native sequence | 3.33 |
| 34 | E6K, K53A, S55A | 272.5 |
| 39 | E6K, K53A | 0.72 |
| 42 | E6K, K53A, S55A, T63A | 0.79 |
| 50 | E6K, K53A, T63A | 1.77 |
| 54 | E6K, C38S, K53A, C68S, K70C, C76S, | 9.12 |
| | C127S | |
| 56 | E6K, K53A, C38S, C76S, C127S | 3.73 |
| 57 | E6K, C38S, K53A | 0.86 |
| 30 | E6K, V11I, C38A, K53A, T63A, C76A, | 0.034 |
| | C127A | |
| 62 | E6K, C38A, K53A, C127A | 0.17 |
| 60 | E6K, C38Q, K53A | 0.203 |
| 59 | E6K, C38A, K53A | 0.268 |
| 57 | E6K, C38S, K53A | 0.53 |
| C143 | V11I, C38A, K53A, C76A, C127A | 0.98 |
| C144 | V11I, C38A, K53A, T63A, C76A, C127A | 0.17 |
| C145 | V11I, C38A, K53A, S55A, C76A, C127A | 3.63 |
| C146 | V11I, C38A, M51G, K53A, C76A, C127A | 0.8 |
| C147 | V11I, C38A, K53A, D54A, C76A, C127A | 1 |
| C148 | F2A, V11I, C38A, K53A, C76A, C127A | 7.28 |
| C149 | V11I, E31A, C38A, K53A, C76A, C127A | 6.6 |
| C150 | V11I, T34A, C38A, K53A, C76A, C127A | 0.7 |
| C151 | V11I, D35A, C38A, K53A, C76A, C127A | 13.12 |
| C152 | V11I, S36A, C38A, K53A, C76A, C127A | 0.25 |
| C153 | V11I, D37A, C38A, K53A, C76A, C127A | 14.12 |
| C154 | V11I, E31A, D37A, C38A, K53A, C76A, | 11.95 |
| | C127A | |
| C155 | V11I, C38A, D40A, K53A, C76A, C127A | 0.52 |
| C156 | V11I, C38A, N41A, K53A, C76A, C127A | 11.7 |
| C157 | V11I, C38A, K53A, C76A, C127A, D132A | 1.95 |
| C158 | V11I, C38A, K53A, C76A, G108A, C127A | 15.56 |
| C159 | V11I, C38A, K53A, C76A, H109A, C127A | 19.5 |
| C160 | V11I, C38A, K53A, C76A, D110A, C127A | 2.02 |
| C161 | K8R, V11I, C38A, C76A, Q103E, C127A | 2.01 |
| C162 | K8E, V11I, C38A, C76A, Q103R, C127A | 2.3 |
| C163 | V11I, C38A, C76A, Q103K, C127A | 1.5 |
| C164 | V11I, C38A, S55H, C76A, C127A | 3.14 |
| C165 | V11I, C38A, S55R, C76A, C127A | 1.91 |
| C166 | V11I, C38A, S55T, C76A, C127A | 4.73 |
| C167 | V11I, C38A, C76A, S105I, C127A | 5.37 |
| C168 | V11I, C38A, C76A, S105K, C127A | 7.73 |
| C174 | K8L, E6K, V11I, C38A, K53A, T63A, | 0.29 |
| | C76A, C127A | |
| C176 | E6K, V11I, C38A, I49M, K53A, T63A, | 0.07 |
| | C76A, C127A | |
| C177 | E6K, V11I, C38A, I49R, K53A, T63A, | 0.04 |
| | C76A, C127A | |
| C178 | E6K, V11I, C38A, K53A, T63A, C76A, | 0.26 |
| | Q103R, C127A | |
| C179 | E6K, K8E, V11I, C38A, K53A, T63A, | 0.4 |
| | C76A, Q103R, C127A | |
| C181 | E6K, V11I, C38A, K53A, T63A, C76A, | 0.1 |
| | C127A, V153E | |
| C182 | E6K, V11I, C38A, K53A, T63A, C76A, | 0.08 |
| | C127A, V153Y | |
| C183 | E6K, V11I, C38A, M51G, K53A, T63A, | 0.1 |
| | C76A, C127A | |
| C184 | E6R, V11I, C38A, K53A, T63A, C76A, | 0.04 |
| | C127A | |
| C140 | E6K, V11I, C38A, K53A, T63A, C76A, | 2.5 |
| | C127A | |
| C141 | E6K, V11I, C38A, K53A, T63A, C76A, | 1.68 |
| | C127A | |
| C142 | Y1M, E6K, V11I, C38A, K53A, T63A, | 0.02 |
| | C76A, C127A | |
| C192 | E6K, V11I, C38A, M51G, K53A, T63A, | 13.99 |
| | C76A, C127A | |

17B-IL-18 BP AlphaLISA assay-An IL-18 binding protein AlphaLISA experiment substantially as described in Example 4 was performed on IL-18 polypeptide which can be incorporated into immunocytokine compositions as provided herein to assess ability to bind to IL-18BP. Results are shown in the Table below. It is expected that the IL-18 polypeptides provided below would behave similarly to C086 (SEQ ID NO: 30) when incorporated into an immunocytokine composition (i.e., a conjugate immunocytokine or fusion immunocytokine) as those otherwise provided herein (e.g., the relative activity of the IL-18 polypeptide in the immunocytokine will be the same).

| SEQ ID NO: or | | |
|---|---|---|
| Composition | | KD |
| ID | Sequence modifications | (nM) |
| 1 | Native Sequence | 0.67 |
| 34 | E06K, K53A, S55A | >1500 |
| 35 | Y01G, F02A, E06K, M51G, K53A, | 969.0 |
| | D54A, S55A, T63A | |
| 36 | K53A | 513.8 |
| 37 | S55A | 10.7 |
| 38 | E06K | 0.13 |
| 39 | E06K, K53A | 130.3 |
| 40 | E06K, S55A | 12.3 |
| 41 | K53A, S55A | 500.0 |
| 42 | E06K, K53A, S55A, T63A | 822.0 |
| 43 | E06K, K53A, S55A, Y01G | |
| 44 | E06K, K53A, S55A, F02A | >1000 |
| 45 | E06K, K53A, S55A, D54A | >1000 |
| 46 | E06K, K53A, S55A, M51G | >1000 |
| 47 | C38S, C68S, C76S, C127S | 0.03 |
| 48 | C38S, C68S, C76S, C127S, K70C | 0.21 |
| 49 | E06K, K53A, S55A, C38S, C68S, C76S, | >1000 |
| | C127S, K70C | |
| 50 | E06K, K53A, T63A | 339.8 |
| 51 | T63A | 2.59 |
| 52 | E06K, T63A | 0.83 |
| 53 | K53A, T63A | 198 |
| 54 | E06K, K53A, C38S, C68S, C76S, C127S, | 446.0 |
| | K70C | |
| 55 | K53A, T63A, C38S, C68S, C76S, C127S, | 913 |
| | K70C | |
| 56 | E6K, K53A, C38S, C76S, C127S | 435.5 |
| 57 | E6K, K53A, C38S | 50.2 |
| C143 | V11I, C38A, K53A, C76A, C127A | 8.86 |
| C144 | V11I, C38A, K53A, T63A, C76A, C127A | 0.66 |
| C145 | V11I, C38A, K53A, S55A, C76A, C127A | 9.74 |
| C146 | V11I, C38A, M51G, K53A, C76A, C127A | 373.30 |
| C147 | V11I, C38A, K53A, D54A, C76A, C127A | 25.77 |
| C148 | F2A, V11I, C38A, K53A, C76A, C127A | 57.21 |
| C149 | V11I, E31A, C38A, K53A, C76A, C127A | 0.64 |
| C150 | V11I, T34A, C38A, K53A, C76A, C127A | 1.24 |
| C151 | V11I, D35A, C38A, K53A, C76A, C127A | 2.88 |
| C152 | V11I, S36A, C38A, K53A, C76A, C127A | 1.12 |
| C153 | V11I, D37A, C38A, K53A, C76A, C127A | 4.55 |
| C154 | V11I, E31A, D37A, C38A, K53A, C76A, | 2.12 |
| | C127A | |
| C155 | V11I, C38A, D40A, K53A, C76A, C127A | 0.74 |
| C156 | V11I, C38A, N41A, K53A, C76A, C127A | 18.47 |
| C157 | V11I, C38A, K53A, C76A, C127A, D132A | 13.70 |
| C158 | V11I, C38A, K53A, C76A, G108A, C127A | 1.24 |
| C159 | V11I, C38A, K53A, C76A, H109A, C127A | 0.55 |
| C160 | V11I, C38A, K53A, C76A, D110A, C127A | 0.71 |
| C161 | K8R, V11I, C38A, C76A, Q103E, C127A | 0.06 |
| C162 | K8E, V11I, C38A, C76A, Q103R, C127A | 0.85 |
| C163 | V11I, C38A, C76A, Q103K, C127A | 0.05 |
| C164 | V11I, C38A, S55H, C76A, C127A | 0.08 |
| C165 | V11I, C38A, S55R, C76A, C127A | 0.15 |
| C166 | V11I, C38A, S55T, C76A, C127A | 0.02 |
| C167 | V11I, C38A, C76A, S105I, C127A | 0.04 |
| C168 | V11I, C38A, C76A, S105K, C127A | 0.05 |
| C174 | K8L, E6K, V11I, C38A, K53A, T63A, | 0.14 |
| | C76A, C127A | |
| C176 | E6K, V11I, C38A, I49M, K53A, T63A, | 25.84 |
| | C76A, C127A | |
| C177 | E6K, V11I, C38A, I49R, K53A, T63A, | >2800 |
| | C76A, C127A | |
| C178 | E6K, V11I, C38A, K53A, T63A, C76A, | >2800 |
| | Q103R, C127A | |
| C179 | E6K, K8E, V11I, C38A, K53A, T63A, | >2800 |
| | C76A, Q103R, C127A | |
| C180 | E6K, V11I, C38A, K53A, T63A, C76A, | |
| | C127A, V153R | |
| C181 | E6K, V11I, C38A, K53A, T63A, C76A, | >2800 |
| | C127A, V153E | |
| C182 | E6K, V11I, C38A, K53A, T63A, C76A, | >2800 |
| | C127A, V153Y | |
| C183 | E6K, V11I, C38A, M51G, K53A, T63A, | >2800 |
| | C76A, C127A | |
| C184 | E6R, V11I, C38A, K53A, T63A, C76A, | 5.46 |
| | C127A | |
| C140 | E6K, V11I, C38A, K53A, T63A, C76A, | >2800 |
| | C127A | |
| C141 | E6K, V11I, C38A, K53A, T63A, C76A, | >2800 |
| | C127A | |
| C142 | Y1M, E6K, V11I, C38A, K53A, T63A, | 2.25 |
| | C76A, C127A | |
| C192 | E6K, V11I, C38A, M51G, K53A, T63A, | >2800 |
| | C76A, C127A | |
| 62 | E6K, C38A, K53A, C127A | 69.62 |
| 60 | E6K, C38Q, K53A | 24.8 |
| 59 | E6K, C38A, K53A | 35.95 |

17C-IFNγ Stimulation and IL-18BP Inhibition Assay-The experiments described in Example 4 for determination of IFNg stimulation in NK92 cells (and inhibition by IL-18 BP) were performed substantially as described on IL-18 polypeptides in order to assess their activities and their suitability for incorporation into immunocytokine compositions. Results are shown in the table below. It is expected that the IL-18 polypeptides provided below would behave similarly to C086 (SEQ ID NO: 30) when incorporated into an immunocytokine composition (i.e., a conjugate immunocytokine or fusion immunocytokine) as those otherwise provided herein (e.g., the relative activity of the IL-18 polypeptide in the immunocytokine will be the same).

| SEQ ID NO: or | | IC₅₀ | EC₅₀ |
|---|---|---|---|
| Composition ID | Sequence modifications | (nM) | (nM) |
| 1 | Native sequence | 1.47 | 0.276 |
| 34 | E06K, K53A, S55A | 229 | 0.824 |
| 35 | Y01G, F02A, E06K, M51G, K53A, D54A, S55A, T63A | >55.0 | >55.0 |
| 36 | K53A | 27.3 | 0.444 |
| 37 | S55A | 4.46 | 0.108 |
| 38 | E06K | 7.79 | 0.0567 |
| 39 | E06K, K53A | >703 | 0.0192 |
| 40 | E06K, S55A | 15 | 0.067 |
| 41 | K53A, S55A | 37.3 | 1.58 |
| 42 | E06K, K53A, S55A, T63A | 1060 | 0.144 |
| 43 | E06K, K53A, S55A, Y01G | 27.8 | 6.12 |
| 44 | E06K, K53A, S55A, F02A | NT | >1000 |
| 45 | E06K, K53A, S55A, D54A | NT | 30 |
| 46 | E06K, K53A, S55A, M51G | 0.189 | 7.4 |
| 47 | C38S, C68S, C76S, C127S | 0.444 | 0.115 |
| 48 | C38S, C68S, C76S, C127S, K70C | 0.114 | 0.488 |
| 49 | E06K, K53A, S55A, C38S, C68S, C76S, C127S, K70C | NT | 58.5 |
| 50 | E06K, K53A, T63A | >1000 | 0.0268 |
| 51 | T63A | 0.239 | 0.449 |
| 52 | E06K, T63A | 47.1 | 0.011 |
| 53 | K53A, T63A | 18.2 | 0.155 |
| 54 | E06K, K53A, C38S, C68S, C76S, C127S, K70C | 23.5 | 0.962 |
| 55 | K53A, T63A, C38S, C68S, C76S, C127S, K70C | >1000 | 17.2 |
| 6 | E6K, V11I, C38A, K53A, T63A, C68A, C76A, C127A, | 5.847 | 1.366 |
| | D98C | | |
| 5 | E6K, V11I, C38A, K53A, T63A, C68A, C76A, C127A, | 62.37 | 0.075 |
| | M86C | | |
| 9 | E6K, C38A, K53A, C68A, D98C | 960.8 | 0.069 |
| 4 | E6K, C38A, K53A, C68A, M86C | 396.3 | 0.022 |
| 62 | E6K, C38A, K53A, C127A | 283.6 | 0.026 |
| 60 | E6K, C38Q, K53A | 780.5 | 0.006 |
| 59 | E6K, C38A, K53A | 653.5 | 0.015 |
| 57 | E6K, C38S, K53A | 146.2 | 0.045 |
| C143 | V11I, C38A, K53A, C76A, C127A | 1.625 | 0.138 |
| C144 | V11I, C38A, K53A, T63A, C76A, C127A | 7.522 | 0.012 |
| C145 | V11I, C38A, K53A, S55A, C76A, C127A | 10.24 | 0.087 |
| C146 | V11I, C38A, M51G, K53A, C76A, C127A | 732.9 | 0.037 |
| C147 | V11I, C38A, K53A, D54A, C76A, C127A | 47.63 | 0.079 |
| C148 | F2A, V11I, C38A, K53A, C76A, C127A | 5.055 | 0.256 |
| C149 | V11I, E31A, C38A, K53A, C76A, C127A | 1.167 | 0.187 |
| C150 | V11I, T34A, C38A, K53A, C76A, C127A | 21.27 | 0.015 |
| C151 | V11I, D35A, C38A, K53A, C76A, C127A | 3.622 | 0.061 |
| C152 | V11I, S36A, C38A, K53A, C76A, C127A | 7.85 | 0.033 |
| C153 | V11I, D37A, C38A, K53A, C76A, C127A | 2.222 | 0.175 |
| C154 | V11I, E31A, D37A, C38A, K53A, C76A, C127A | 3.709 | 0.062 |
| C155 | V11I, C38A, D40A, K53A, C76A, C127A | 3.233 | 0.067 |
| C156 | V11I, C38A, N41A, K53A, C76A, C127A | 0.681 | 0.558 |
| C157 | V11I, C38A, K53A, C76A, C127A, D132A | 6.082 | 0.056 |
| C158 | V11I, C38A, K53A, C76A, G108A, C127A | 3.981 | 0.073 |
| C159 | V11I, C38A, K53A, C76A, H109A, C127A | 1.807 | 0.123 |
| C160 | V11I, C38A, K53A, C76A, D110A, C127A | 3.181 | 0.028 |
| C161 | K8R, V11I, C38A, C76A, Q103E, C127A | 1.073 | 0.057 |
| C162 | K8E, V11I, C38A, C76A, Q103R, C127A | 7.292 | 0.061 |
| C163 | V11I, C38A, C76A, Q103K, C127A | 0.823 | 0.093 |
| C164 | V11I, C38A, S55H, C76A, C127A | 0.456 | 0.414 |
| C165 | V11I, C38A, S55R, C76A, C127A | 0.885 | 0.176 |
| C166 | V11I, C38A, S55T, C76A, C127A | 0.44 | 0.098 |
| C167 | V11I, C38A, C76A, S105I, C127A | 0.809 | 0.103 |
| C168 | V11I, C38A, C76A, S105K, C127A | 0.176 | 0.098 |

### Example 18: Immune Cell Associated Antigen Binding ELISA Assay for Fusion Immunocytokines

The interaction of the unmodified antibodies and corresponding IL-18 immunocytokines with relevant immune cell associated antigen are measured by ELISA assay. For these studies, Corning high-binding half-area plates (Fisher Scientific, Reinach, Switzerland) are coated overnight at 4° C. with 25 μl of unmodified antibodies corresponding IL-18 immunocytokines at 5 μg/ml in PBS. Plates are then washed four times with 100 μl of PBS-0.02% Tween20. Plate surfaces are blocked with 25 μl of PBS-0.02% Tween20-1% BSA at 37° C. during 1h. Plates are then washed four times with 100 μl of PBS-0.02% Tween20.Twenty-five microliters (25 μl) of recombinant biotinylated human PD-1 (Biotinylated Recombinant Human PD-1/CD279-Fc Chimera,carrier-free, Biolegend #789406) protein are added in seven-fold serial dilutions starting at 12 nM down to 0.15 μM into PBS-0.02%Tween20-0.1% BSA and incubated at 37° C. during 2h. Plates are then washed four times with 100 μl of PBS-0.02% Tween20. Twenty-five microliters of Streptavidin-Horseradish peroxidase (#RABHRP3, Merck, Buchs, Switzerland) diluted at 1:500 into PBS-0.02%Tween20-0.1% BSA are added to each well and incubated at Room Temperature during 30 min. Plates are then washed four times with 100 μl of PBS-0.02% Tween20. Fifty microliters of TMB substrate reagent (#C_{L}07, Merck, Buchs, Switzerland) are added to each well and incubated at 37° C. during 5 min. After 5 min at 37° C., Horseradish peroxidase reaction is stopped by adding 50 μl/well of 0.5M H2SO₄ stop solution. ELISA signal is then measured at 450 nm on an ENSPIRE® plate reader from Perkin Elmer (Schwerzenbach, Switzerland). Results from this experiment are shown in the table below.

KD values of the interaction of fusion immunocytokines
with human PD-1 as measured by ELISA

| Antibody | PD-1 KD (pM) |
|---|---|
| Pembrolizumab/Keytruda | |
| LZM-009 | 40.7 |
| Nivolumab/Opdivo | 91.9 |
| Durvalumab/Imfinzi | >100000 |
| Atezolizumab/Tecentriq | >100000 |
| Avelumab/Bavencio | >100000 |
| CMP2001 | 38.9 |
| CMP2002 | 70.2 |
| CMP2003 | NT |
| CMP2004 | NT |
| CMP2005 | NT |
| CMP2006 | NT |
| CMP2007 | NT |

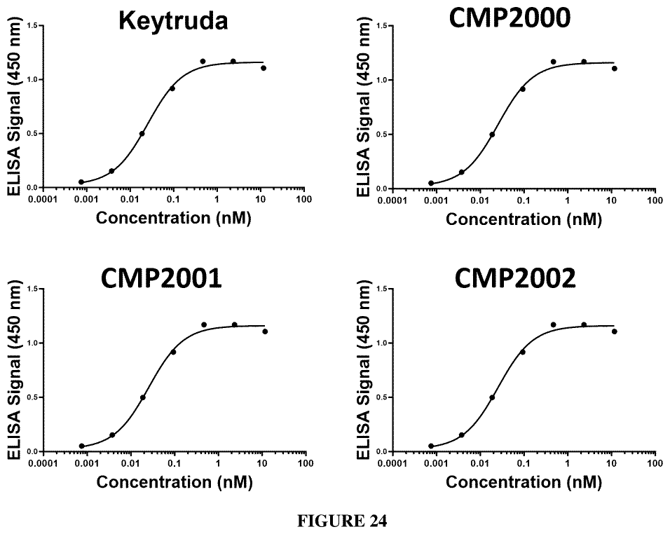

FIG. 24 show plots measuring ability of the unmodified and of fused anti-PD1 antibodies to bind with human PD1/CD279 ligand, with the figure showing ELISA signal on the y-axis and dosage of the biotinylated PD-1 protein on the x-axis. The unconjugated reference antibodies are Pembrolizumab/Keytruda and LZM-009 (CMP2000). The IL-18 fused antibodies tested in this figure are CMP2001 and CMP2002

### Example 19-Kinetic Analysis of Binding of reference antibodies and Fusion Immunocytokines to Immune Cell Associated Antigens

Based on Bio-Layer Interferometry (BLI), Octet® BLI systems enable real-time, label-free analysis for the determination of kinetics and affinity of a ligands to its receptor. Here anti-human IgG FC Capture (AHC) sensors are loaded with the test items (ICs). Sensors are first dipped into a kinetic buffer for baseline measurement, then into an analyte solution, here human PD1, to allow association and again into a buffer solution where the analyte is allowed to come off the ligand (dissociation). Several concentrations of analyte are run in parallel and enable the calculation of affinity parameters: Ka, Kd, KD.

Typically, first, the sensors are regenerated by 3 cycles of dipping into 10 mM glycine solution at pH=2 for 20 seconds, followed by 20 second kinetics buffer and a final 60 seconds in kinetics buffer to establish the initial signal (baseline). Second, the loading column will contain the ligand, here the unmodified PD-1 antibodies and of IL-18 polypeptide conjugated PD-1 antibody, at a fixed concentration determined in the loading scout experiment (20 μg/mL). Then another wash/baseline step allows non immobilized proteins to be washed away. The association column will contain the 2-fold dilution series of the analyte (His-tagged human PD1, R&D #8986-PD) including a no analyte control. The highest concentration should be ˜10-fold the KD. The dissociation designates the sensors to return to previous baseline column with kinetics buffer. After acquisition, the data is analyzed with Data Analysis Studio software (Sartorius). Data sets are first preprocessed by subtracting references samples and aligning curves on the baseline step. Group fitting is then applied to the data series and kinetics parameters are calculated. Results from this experiment are shown in the table below.

Binding kinetics of the interaction of reference
antibodies and fusion immunocytokines with PD-1
as measured by Bio-Layer Interferometry (BLI)

| | ka | kd | KD |
|---|---|---|---|
| Antibody | (1/Ms) | (1/s) | (nM) |
| Pembrolizumab/Keytruda | 3.73E+05 | 2.46E−03 | 6.7 |
| LZM-009 | 3.42E+05 | 7.70E−03 | 23.50 |
| CMP2001 | NT | NT | NT |
| CMP2002 | 2.30E+05 | 8.95E−03 | 39 |
| CMP2003 | NT | NT | NT |
| CMP2004 | NT | NT | NT |
| CMP2005 | NT | NT | NT |
| CMP2006 | NT | NT | NT |
| CMP2007 | NT | NT | NT |

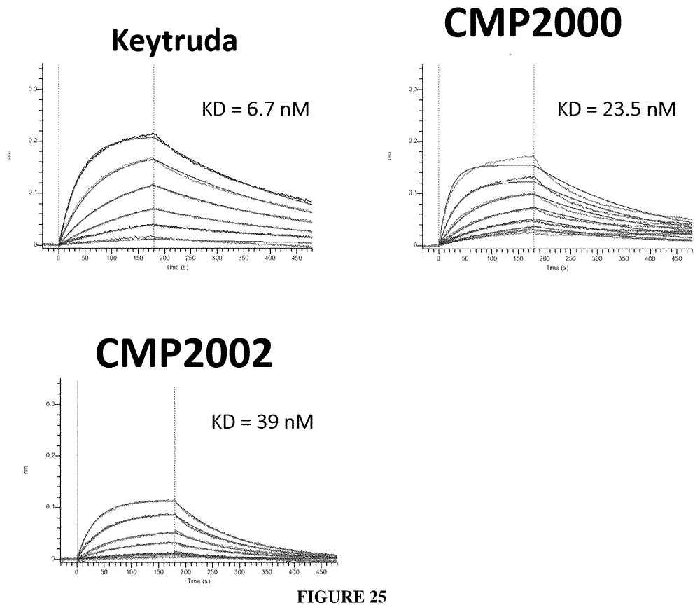

FIG. 25 shows plots measuring ability of the unmodified and of fused antibodies to bind to human PD-L1/B7-H1 ligand, with the figure showing net BioLayer interferometry shift in nanometer on the y-axis and time of incubation dosage of the biotinylated PD-1 protein on the x-axis. The reference antibodies are Pembrolizumab/Keytruda and LZM-009 (CMP2000). The IL-18 fused antibody tested in this figure is CMP2002.

### Example 20-Human FcRn Binding Assay for Fusion Immunocytokines

The interaction of the unmodified and of conjugated anti-PD1 antibodies with the human neonatal Fc receptor (FcRn) at pH 6 was measured using the AlphaLISA® Human FcRn Binding Kit (AL3095C) from Perkin Elmer (Schwerzenbach, Switzerland). The AlphaLISA® detection of FcRn and IgG binding uses IgG coated AlphaLISA® acceptor beads to interact with biotinylated human FcRn captured on Streptavidin-coated donor beads. When reference IgG binds to FcRn, donor and acceptor beads come into proximity enabling the transfer of singlet oxygen that trigger a cascade of energy transfer reactions in the acceptor beads, resulting in a sharp peak of light emission at 615 nm. Addition of a free IgG antibodies into the AlphaLISA® mixture creates a competition for the binding of FcRn to the reference antibody resulting in a loss of signal.

Briefly, test molecules were measured in serial dilutions starting at 5 μM down to 64 μM and incubated with AlphaLISA® reaction mixture consisting of 800 nM of recombinant biotinylated human FcRn, 40 μg/ml of human IgG conjugated Acceptor beads, and 40 μg/ml of Streptavidin coated Donor beads in pH 6 MES buffer. After 90 min at 23° C. in the dark, AlphaLISA® signal was measured on an EnSpire plate reader (Excitation at 680 nm, Emission at 615 nm) from Perkin Elmer (Schwerzenbach, Switzerland). Results from this experiment are shown in the table below.

Binding affinity of reference antibodies and fusion immunocytokines
with the human neonatal Fc receptor as measured by AlphaLISA

| Antibody | Human FcRn KD (nM) |
|---|---|
| Pembrolizumab/Keytruda | |
| LZM-009 | 6.00 |
| Durvalumab/Imfinzi | 8.31 |
| Atezolizumab/Tecentriq | 13.03 |
| Avelumab/Bavencio | 5.36 |
| CMP2001 | 12.51 |
| CMP2002 | 4.68 |
| CMP2003 | NT |
| CMP2004 | NT |
| CMP2005 | NT |
| CMP2006 | NT |
| CMP2007 | NT |

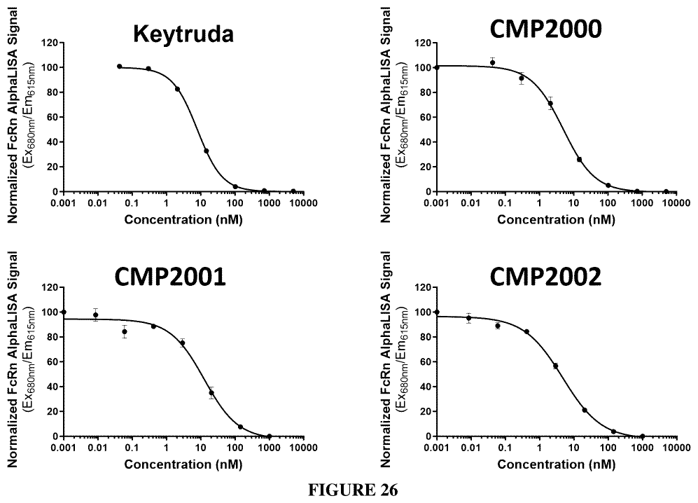

FIG. 26 shows plots measuring ability of the unmodified and of fused antibodies to bind to human Fc neonatal receptor. The figure shows normalized AlphaLISA signal on the y-axis and dosage of the human Fc neonatal receptor (FcRn) on the x-axis. The reference antibodies are Pembrolizumab/Keytruda and LZM-009 (CMP2000). The IL-18 fused antibodies tested in this figure are CMP2001 and CMP2002.

### Example 21-HEK-Blue IL18R Reporter Assay for Fusion Immunocytokines

An IL-18R positive HEK-Blue reporter cell line is used to determine binding of IL-18 variants to IL-18R and subsequent downstream signaling. The general protocol is outlined below.

5×10⁴ cells HEK-Blue IL18R reporter cells (InvivoGen, #hkb-hmil18) are seeded into each well of a 96 well plate and stimulated with 0-100 nM of IL-18 polypeptide variants at 37° C. and 5% CO2. After 20h incubation, 20 μL of cell culture supernatant is then taken from each well and mixed with 180 μL QUANTI-Blue media in a 96 well plate, incubated for 1 hour at 37° C. and 5% CO2. The absorbance signal at 620 nm is then measured on an Enspire plate reader. Half Maximal Effective dose (EC₅₀) is calculated based on a variable slope, four parameter analysis using GraphPad PRISM software.

Results of this experiment for select variants are shown in the Table below.

EC₅₀ in HEK-Blue IL18R Reporter Assay Data

| Composition | IL-18R Hek Blue assay EC₅₀ (pM) |
|---|---|
| SEQ ID NO: 1 | 2.35 |
| SEQ ID NO: 30 | 0.057 |
| CMP2001 | >200 |
| CMP2002 | 0.0049 |
| CMP2003 | NT |
| CMP2004 | NT |
| CMP2005 | NT |
| CMP2006 | NT |
| CMP2007 | NT |

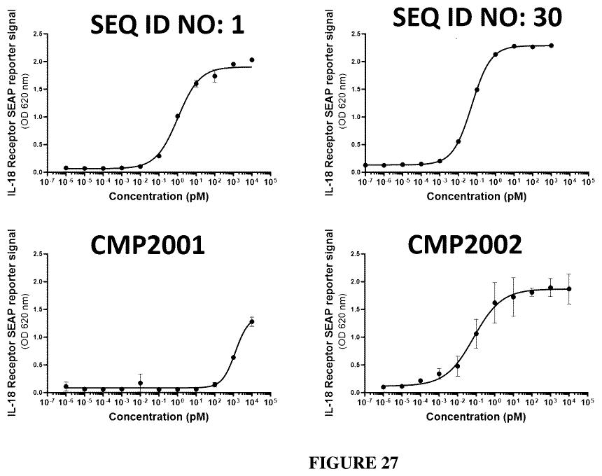

FIG. 27 shows plots measuring ability of wild type IL-18 and of modified IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (OD 620 nm) on the y-axis, and dosage of the IL-18 polypeptides on the x-axis. The IL-18 polypeptides are native IL-18 wild-type (SEQ ID NO:1), SEQ ID NO: 30,CMP2001, and CMP2002.

### Example 22: IFNγ Induction on primary human cells by fusion immunocytokines

Ability of IL-18 variants to stimulate Human peripheral blood mononuclear cells (PBMCs) was assessed according to the following protocol.

Isolation of lymphocytes: Blood from Buffy Coats of healthy volunteers was diluted with equal volume of PBS and slowly poured on top of SepMate tube prefilled with 15 mL Histopaque-1077. Tubes were centrifuged for 10 minutes at 1200g, the top layer was collected and washed 3 times with PBS containing 2% of Fetal Bovine Serum. PBMCs were counted and cryopreserved as aliquots of 20× 10⁶ cells.

Cryopreserved PBMCs were thawed and seeded at 150 000 cells/well in a 96w round bottom 96 well plate. PBMCs were stimulated with a gradient of human IL-18 variants ranging from 0.2 pg/mL to 3600 ng/mL. All stimulations were performed in the presence of hIL-12 (1 ng/ml, Sino Biological, #CT011-H08H) for 24 hrs in RPMI containing 10% Fetal Bovine Serum.

Cytokine production after 24 hr stimulation were measured using Legendplex bead-based cytokine assay (Biolegend #740930) according to manufacturer protocol. Half maximal effective concentrations (EC₅₀) of IFNg released in culture supernatant were calculated based on a variable slope and four parameter analysis using GraphPad PRISM software.

EC₅₀ in PBMC IFNg secretion Assay

| Antibody | IFNγ secretion PBMCs EC₅₀ (nM) |
|---|---|
| Pembrolizumab/Keytruda | |
| LZM-009 | 40.7 |
| SEQ ID NO: 1 | 0.698 |
| SEQ ID NO: 30 | 0.0022 |
| CMP2001 | >200 |
| CMP2002 | 0.0049 |
| CMP2003 | NT |
| CMP2004 | NT |
| CMP2005 | NT |
| CMP2006 | NT |
| CMP2007 | NT |

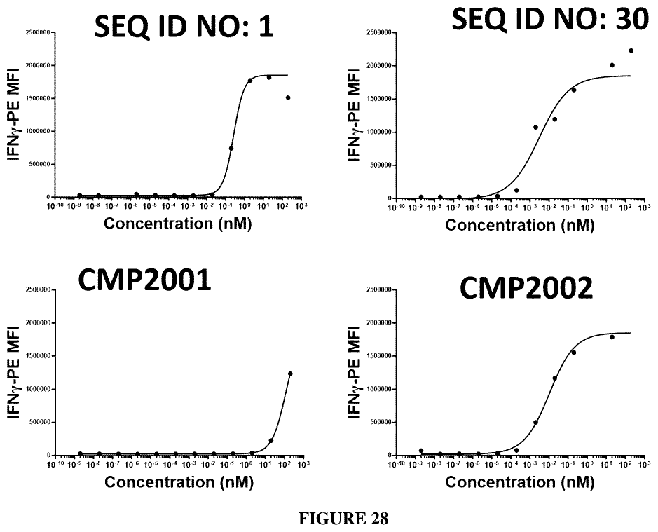

FIG. 28 shows plots measuring ability of wild type IL-18 and of modified IL-18 polypeptides to stimulate the secretion of IFNgamma by human Peripheral Blood Mononuclear Cells (PBMCs). The figure shows mean IFNg signal on the y-axis and dosage of the IL-18 polypeptides on the x-axis. The IL-18 polypeptides are native IL-18 wild-type (SEQ ID NO:1), SEQ ID NO: 30, CMP2001 and, CMP2002

### Example 23-IL-18BP Binding alphaLISA Assay for Fusion Immunocytokines

A human IL-18BP AlphaLISA Assay Kit is used to determine the binding affinity of each IL-18 variant for IL-18BP, which detected the presence of free form IL-18BP.

Sixteen three-fold serial dilutions of IL-18 analytes are prepared in aMEM medium supplemented with 20% FCS, Glutamax, and 25 μM B-mercaptoethanol in the presence of 5 ng/ml of His-tagged human IL-18BP. Final IL-18 analytes concentration range from 2778 nM to 0.2 μM.

After 1 hr incubation at room temperature, free IL-18BP levels are measured using a Human IFNγ AlphaLISA Assay Kit. In a 384 well OPTIplate, 5 μL of 5X Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (K_{D}) is calculated based on a variable slope, four parameter analysis using GraphPad PRISM software.

The table below shows results of the dissociation constants (K_{D}) observed for the IL-18 variants described to IL-18BP as measured using the protocol described in Example 10.

Binding of IL-18BP monomer determined
by alphaLISA to fusion immunocytokines

| Composition | KD (nM) |
|---|---|
| Pembrolizumab/Keytruda | |
| LZM-009 | 40.7 |
| SEQ ID NO: 1 | 0.698 |
| SEQ ID NO: 30 | 0.0022 |
| CMP2001 | >200 |
| CMP2002 | 0.0049 |
| CMP2003 | NT |
| CMP2004 | NT |
| CMP2005 | NT |
| CMP2006 | NT |
| CMP2007 | NT |

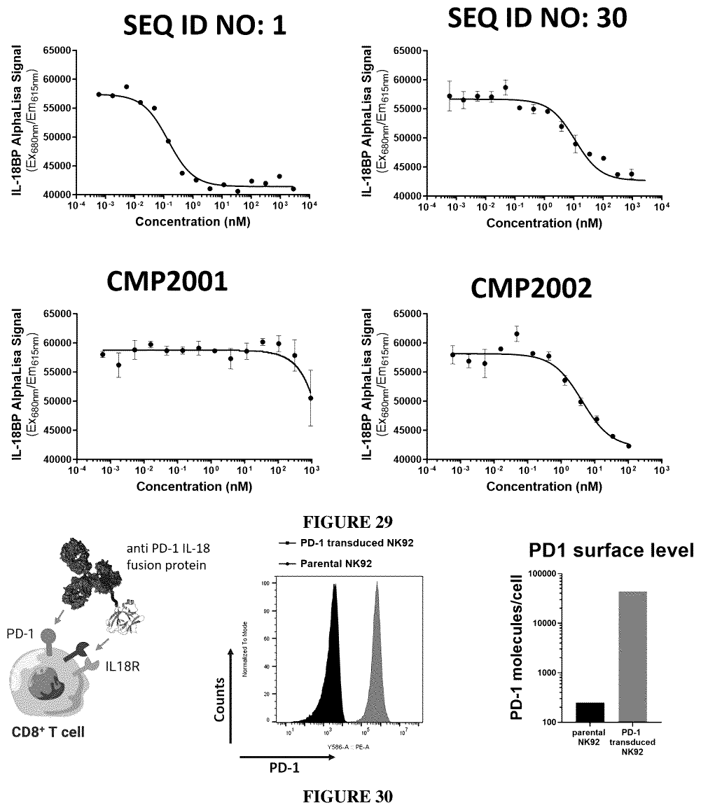

FIG. 29 shows plots measuring the ability of wild type IL-18 and of modified IL-18 polypeptides to bind to the human IL-18 Binding Protein (IL-18BP). The figure shows free IL-18BP AlphaLISA signal on the y-axis, and dosage of IL-18 polypeptides on the x-axis. The IL-18 polypeptides are native IL-18 wild-type (SEQ ID NO:01), SEQ ID NO: 30, CMP2001 and, CMP2002.

### Example 24: IFN Gamma Secretion Assay in PD-1^{negative} and PD-1^{positive} NK-92 Cells by Fusion Immunocytokines

To show selectivity of the anti-PD1-IL18 immunocytokine (IC) for PD-1 positive cells, the NK-92 cell line was used to generate a PD-1 expressing cell line and furthermore, to measure IFNγ release upon incubation with IL-18 variants and ICs.

On the day of experiment, parental PD-1^{negative} and PD-1^{positive} transduced cells were harvested and washed with PBS 1× and resuspended in medium (w/o IL-2) containing 1 ng/ml of recombinant human Interleukin-12 (SinoBiologicals, Cat #CT011-H08H). After counting, cells were seeded at 100 000 cells/well in a 384 well culture plate and incubated at 37° C./5% CO2. When indicated, cells were preincubated with anti-PD-1 antibody LZM-009 at 17 μM during 30 min at 37° C. The test items were diluted to 200 nM in culture medium, followed by twelve 5-fold serial dilutions. The lowest concentration assessed was 0.8 fM.

After 16-20h incubation at 37° C./5% CO2, 5 μl of supernatant were carefully transferred to a 384 microwell OptiPlate (Perkin Elmer; Cat #6007270) and Interferon-gamma (IFNY) levels were measured using the Human IFNγ AlphaLISA Assay Kit (Perkin Elmer, Cat #AL217C). Briefly, 10 μl of 2.5× AlphaLISA Anti-IFNγ acceptor beads and biotinylated Antibody Anti-IFNγ mix were added to the 5 μl of NK-92 supernatants and incubated for 1 h at room temperature while shaking. Under subdued light, 2.5 μl of 2× streptavidin (SA) donor beads were pipetted in each well and incubated for 30 min at room temperature under shaking. AlphaLISA signal was then measured on an Enspire plate reader (Perkin Elmer) using 680 and 615 nm as excitation and emission wavelengths respectively. Half maximal effective concentration (EC₅₀) was calculated based on a variable slope, four parameter analysis using GraphPad PRISM software.

Stimulation of IFNγ secretion by unconjugated IL-18
variants and corresponding IL-18 immunocytokines in parental
PD-1^{negative} and PD-1^{positive} transduced NK-92 cells

| | | PD-1^{negative} | | PD-1^{neg}/ |
|---|---|---|---|---|
| | | Parental | PD-1^{positive} | PD-1^{pos} |
| | | NK-92 | NK-92 | EC₅₀ |
| Composition | | EC₅₀ (nM) | EC₅₀ (nM) | ratio |
| 2 | SEQ ID NO: 1 | 0.1439 | 0.1298 | 1.4 |
| 3 | SEQ ID NO: 30 | 0.0027 | 0.0029 | 1.0 |
| 4 | CMP2001 | 0.02834 | 0.005076 | 5.6 |
| 5 | CMP2002 | 0.00528 | 0.001828 | 2.9 |
| 6 | CMP2003 | 0.09250 | 0.05767 | 1.6 |
| 7 | CMP2004 | 0.02818 | 0.02409 | 1.2 |
| 8 | CMP2005 | 0.04450 | 0.002251 | 19.8 |
| 9 | CMP2006 | 0.07474 | 0.001245 | 60.0 |
| 10 | CMP2007 | 0.02737 | 0.0008223 | 33.3 |

FIG. 30 shows plots measuring the levels PD-1 surface expression on wild type NK-92 cells and on NK-92 cells transduced with human PD-1.

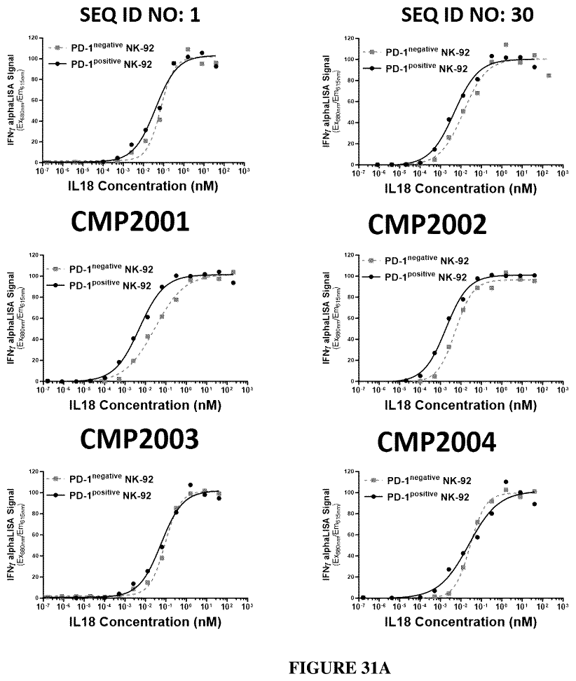

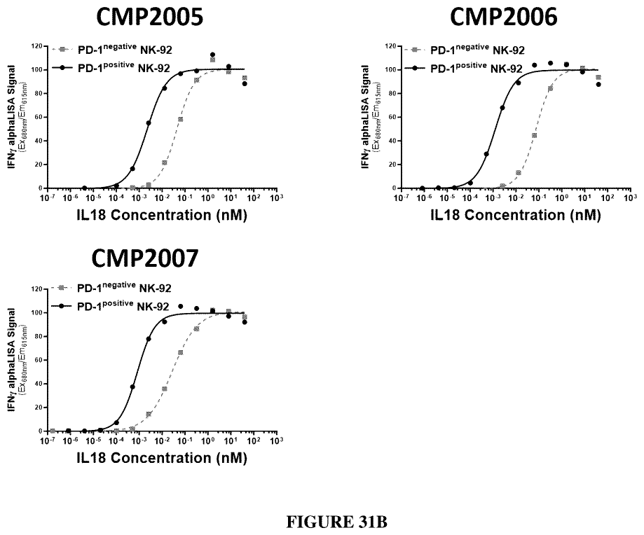

FIG. 31A and FIG. 31B show plots measuring the ability of of wild type IL-18 and of modified IL-18 polypeptides to stimulate the secretion of IFNgamma by parental PD-1^{negative} (grey squared symbols and dotted lines) and by engineered PD-1^{positive} NK-92 cells (black round symbols and plain lines). The figure shows mean IFNg AlphaLISA signal on the y-axis and dosage of the masked IL-18 PD-1 immunocytokines on the x-axis. The IL-18 polypeptides are native IL-18 wild-type (SEQ ID NO:1), SEQ ID NO: 30, CMP2001,CMP2002, CMP2003, CMP2004, CMP2005, CMP2006, and CMP2007.

### Example 25: IL-18BP Mediated Inhibtion of IFN Gamma Secretion Assay in PD-1^{negative} and PD-1^{positive} NK-92 Cells by Fusion Immunocytokines

To characterize the resistance of anti-PD1-IL18 immunocytokines (IC) on PD-1 positive cells, the NK-92 cell line was used to generate a PD-1 expressing cell line and furthermore, to measure the inhibition of IFNγ release upon incubation with IL-18 variants and ICs in the presence of increasing quantities of human IL-18BP.

On the day of experiment, parental PD-1^{negative} and PD-1^{positive} transduced cells were harvested and washed with PBS 1× and resuspended in medium (w/o IL-2) containing 1 ng/ml of recombinant human Interleukin-12 (SinoBiologicals, Cat #CT011-H08H). After counting, cells were seeded at 100 000 cells/well in a 384 well culture plate and incubated at 37° C./5% CO2. When indicated, cells were preincubated with anti-PD-1 antibody LZM-009 at 17 μM during 30 min at 37° C. Sixteen 2-fold serial dilutions of His-tag human IL-18 binding protein isoform a (IL-18BPa; SinoBiologicals #10357-H08H) were prepared in aMEM medium, supplemented with 1 ng/ml IL-12 and 2 nM of each IL-18 variants, and added to the NK-92 cells. Final IL-18 analytes concentration was 1 nM and final IL-18BPa concentrations ranged from 4500 nM down to 5 μM.

After 16-20h incubation at 37° C./5% CO2, 5 μl of supernatant were carefully transferred to a 384 microwell OptilPlate (Perkin Elmer; Cat #6007270) and Interferon-gamma (IFNY) levels measured using the Human IFNγ AlphaLISA Assay Kit (Perkin Elmer, Cat #AL217C). Briefly, 10 μl of 2.5× AlphaLISA Anti-IFNγ acceptor beads and biotinylated Antibody Anti-IFNγ mix were added to the 5 μl of NK-92 supernatants and incubated for 1 h at room temperature while shaking. Under subdued light, 2.5 μl of 2× streptavidin (SA) donor beads were pipetted in each well and incubated for 30 min at room temperature while shaking. AlphaLISA signal was then measured on an Enspire plate reader (Perkin Elmer) using 680 and 615 nm as excitation and emission wavelengths respectively. Half maximal inhibitory concentration (IC₅₀) was calculated based on a variable slope, four parameter analysis using GraphPad PRISM software

IL-18BP-mediated inhibition of IFNγ secretion by unconjugated
IL-18 variants and corresponding IL-18 fusion immunocytokines in
parental PD-1^{negative} and PD-1^{positive} transduced NK-92 cells

| | PD-1^{negative} | | PD-1^{pos}/ |
|---|---|---|---|
| | Parental | PD-1^{positive} | PD-1^{neg} |
| | NK-92 | NK-92 | IC₅₀ |
| BPT # | IC₅₀ (nM) | IC₅₀ (nM) | ratio |
| SEQ ID NO: 1 | 2.97 | 2.67 | 1.2 |
| SEQ ID NO: 30 | 109.54 | 80.32 | 1.0 |
| CMP2001 | NT | NT | NT |
| CMP2002 | NT | NT | NT |
| CMP2003 | NT | NT | NT |
| CMP2004 | NT | NT | NT |
| CMP2005 | NT | NT | NT |
| CMP2006 | NT | NT | NT |
| CMP2007 | NT | NT | NT |

### Example 26: Production and purification of anti-PD1-IL18 IC recombinant formats

### Expression in CHO K1 Cells

cDNAs encoding heavy and lights chains of the anti-PD1-IL18 IC recombinant formats were cloned into evitria's vector system using conventional cloning techniques. Plasmid DNA was prepared under low-endotoxin conditions based on anion exchange chromatography and correctness of the sequences was verified with Sanger sequencing. Suspension-adapted CHO K1 cells were used for expression of the molecules. Cells were first grown in eviGrow medium (a chemically defined, animal-component free, serum-free medium), then transfected with eviFect, evitria's custom-made, proprietary transfection reagent. After transfection cells were grown in eviMake2, an animal-component free, serum-free medium, for two weeks. Supernatants were harvested by centrifugation and sterile-filtered.

### Purification

Expression level of the different molecules was assessed using BLI and Protein A sensors accordingly to the manufacturer's instruction (Sartorius). AmMag protein A magnetic beads (GeneScript) were then used to capture the molecules from the supernatant. Elution was performed using 50 mM acetate, pH4.1 followed by 0.1M glycine, pH3.5. Fractions were neutralized by addition of 5% of 1M Tris-HCl pH8.0 and analyzed by SDS-PAGE and analytical SEC (aSEC). For the molecules with a purity lower than 80% by aSEC, a cation exchange chromatography was performed (HiTrap SP HP 1 ml) using an AKTA-Pure25. A gradient ranging from 0 to a maximum of 500 mM of NaCl (in 50 mM acetate pH5.0) over up to 50CV was used to discriminate the different species. Fractions were then analyzed by SDS-PAGE and analytical SEC (aSEC). The fractions with the highest purity were pooled and the concentration of the molecule was assessed by UV280 nm measurement or/and BCA protein assay (Pierce).

Compositions

| Antibody | | | |
|---|---|---|---|
| or Ag- | | | |
| binding | | | Composition |
| fragment | Chain | Sequence | number |
| LZM009 | HC1 | QVQLVQSGAEVKKPGASVKVSCKASGYTFTSYYMYWV | CMP2000 |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| | | QGTTVTVSSASTKGPSVFPLAPCSRSTSESTAALGCLVK | |
| | | DYFPEPVTVSWNSGALTSGVHTFPAVLQSSGLYSLSSVV | |
| | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLTCLVKGFYPSDIAVEW | |
| | | ESNGQPENNYKTTPPVLDSDGSFFLYSRLTVDKSRWQE | |
| | | GNVFSCSVMHEALHNHYTQKSLSLSLGK (SEQ ID NO: | |
| | | 115) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |
| IL-18 Wild | NA | | SEQ ID NO: 1 |
| type | | | |
| IL-18 | NA | YFGKLKSKLSIIRNLNDQVLFIDQGNRPLFEDMTDSDAR | SEQ ID NO: |
| variant | | DNAPRTIFIISMYADSQPRGMAVAISVKCEKISTLSAENK | 30 |
| | | IISFKEMNPPDNIKDTKSDIIFFQRSVPGHDNKMQFESSSY | |
| | | EGYFLAAEKERDLFKLILKKEDELGDRSIMFTVQNED | |
| | | (SEQ ID NO: 30) | |
| LZM009 | HC1 | QVQLVQSGAEVKKPGASVKVSCKASGYTFTSYYMYWV | CMP2001* |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| (T366W, | | QGTTVTVSSASTKGPSVFPLAPCSRSTSESTAALGCLVK | |
| H435R, | | DYFPEPVTVSWNSGALTSGVHTFPAVLQSSGLYSLSSVV | |
| Y436F) | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLWCLVKGFYPSDIAVEW | |
| | | ESNGQPENNYKTTPPVLDSDGSFFLYSRLTVDKSRWQE | |
| | | GNVFSCSVMHEALHNRFTQKSLSLSLGK (SEQ ID NO: | |
| | | 412) | |
| LZM009 | HC2 | QVQLVQSGAEVKKPGASVKVSCKASGYTFTSYYMYWV | |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| (T366S, | | QGTTVTVSSASTKGPSVFPLAPCSRSTSESTAALGCLVK | |
| L368A, | | DYFPEPVTVSWNSGALTSGVHTFPAVLQSSGLYSLSSVV | |
| Y407V) | | TVPSSSLGTKTYTCNVDHKPSNTKVDKRVESKYGPPCPP | |
| fused to | | CPAPEFLGGPSVFLFPPKPKDTLMISRTPEVTCVVVDVSQ | |
| IL18 (IL18 | | EDPEVQFNWYVDGVEVHNAKTKPREEQFNSTYRVVSV | |
| in bold) | | LTVLHQDWLNGKEYKCKVSNKGLPSSIEKTISKAKGQP | |
| | | REPQVYTLPPSQEEMTKNQVSLSCAVKGFYPSDIAVEW | |
| | | ESNGQPENNYKTTPPVLDSDGSFFLVSRLTVDKSRWQE | |
| | | GNVFSCSVMHEALHNHYTQKSLSLSLGKGGGGSGGGG | |
| | | SGGGGSYFGKLKSKLSIIRNLNDQVLFIDQGNRPLFED | |
| | | MTDSDARDNAPRTIFIISMYADSQPRGMAVAISVKCE | |
| | | KISTLSAENKIISFKEMNPPDNIKDTKSDIIFFQRSVPG | |
| | | HDNKMQFESSSYEGYFLAAEKERDLFKLILKKEDEL | |
| | | GDRSIMFTVQNED (SEQ ID NO: 413) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |
| LZM009 | HC | QVQLVQSGAEVKKPGASVKVSCKASGYTFTSYYMYWV | CMP2002* |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| | | QGTTVTVSSASTKGPSVFPLAPCSRSTSESTAALGCLVK | |
| | | DYFPEPVTVSWNSGALTSGVHTFPAVLQSSGLYSLSSVV | |
| | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLTCLVKGFYPSDIAVEW | |
| | | ESNGQPENNYKTTPPVLDSDGSFFLYSRLTVDKSRWQE | |
| | | GNVFSCSVMHEALHNHYTQKSLSLSLGK (SEQ ID NO: | |
| | | 115) | |
| LZM009 | LC | YFGKLKSKLSIIRNLNDQVLFIDQGNRPLFEDMTDSD | |
| light chain | | ARDNAPRTIFIISMYADSQPRGMAVAISVKCEKISTLS | |
| fused to | | AENKIISFKEMNPPDNIKDTKSDIIFFQRSVPGHDNKM | |
| IL18 (IL18 | | QFESSSYEGYFLAAEKERDLFKLILKKEDELGDRSIM | |
| in bold) | | FTVQNEDGGGGSGGGGSGGGGSEIVLTQSPATLSLSPG | |
| | | ERATISCRASKGVSTSGYSYLHWYQQKPGQAPRLLIYLA | |
| | | SYLESGVPARFSGSGSGTDFTLTISSLEPEDFATYYCQHS | |
| | | RELPLTFGTGTKVEIKRTVAAPSVFIFPPSDEQLKSGTAS | |
| | | VVCLLNNFYPREAKVQWKVDNALQSGNSQESVTEQDS | |
| | | KDSTYSLSSTLTLSKADYEKHKVYACEVTHQGLSSPVT | |
| | | KSFNRGEC (SEQ ID NO: 414) | |
| LZM009 | HC | QVQLVQSGAEVKKPGASVKVSCKASGYTFTSYYMYWV | CMP2003* |
| IgG4 heavy | | RQAPGQGLEWMGGVNPSNGGTNFNEKFKSRVTITADK | |
| chain fused | | STSTAYMELSSLRSEDTAVYYCARRDYRYDMGFDYWG | |
| to IL18 | | QGTTVTVSSASTKGPSVFPLAPCSRSTSESTAALGCLVK | |
| (IL18 in | | DYFPEPVTVSWNSGALTSGVHTFPAVLQSSGLYSLSSVV | |
| bold) | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLTCLVKGFYPSDIAVEW | |
| | | ESNGQPENNYKTTPPVLDSDGSFFLYSRLTVDKSRWQE | |
| | | GNVFSCSVMHEALHNHYTQKSLSLSLGKGGGGSGGGG | |
| | | SGGGGSYFGKLKSKLSIIRNLNDQVLFIDQGNRPLFED | |
| | | MTDSDARDNAPRTIFIISMYADSQPRGMAVAISVKCE | |
| | | KISTLSAENKIISFKEMNPPDNIKDTKSDIIFFQRSVPG | |
| | | HDNKMQFESSSYEGYFLAAEKERDLFKLILKKEDEL | |
| | | GDRSIMFTVQNED (SEQ ID NO: 415) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |
| LZM009 | HC | QVQLVQSGAEVKKPGASVKVSCKASGYTFTSYYMYWV | CMP2004 |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| | | QGTTVTVSSASTKGPSVFPLAPCSRSTSESTAALGCLVK | |
| | | DYFPEPVTVSWNSGALTSGVHTFPAVLQSSGLYSLSSVV | |
| | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLTCLVKGFYPSDIAVEW | |
| | | ESNGQPENNYKTTPPVLDSDGSFFLYSRLTVDKSRWQE | |
| | | GNVFSCSVMHEALHNHYTQKSLSLSLGK (SEQ ID NO: | |
| | | 115) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| fused to | | SLEPEDFATYYCQHSRELPLTFGTGTKVEIKRTVAAPSV | |
| IL18 (IL18 | | FIFPPSDEQLKSGTASVVCLLNNFYPREAKVQWKVDNA | |
| in bold) | | LQSGNSQESVTEQDSKDSTYSLSSTLTLSKADYEKHKVY | |
| | | ACEVTHQGLSSPVTKSFNRGECGGGGSGGGGSGGGGSY | |
| | | FGKLKSKLSIIRNLNDQVLFIDQGNRPLFEDMTDSDA | |
| | | RDNAPRTIFIISMYADSQPRGMAVAISVKCEKISTLSA | |
| | | ENKIISFKEMNPPDNIKDTKSDIIFFQRSVPGHDNKMQ | |
| | | FESSSYEGYFLAAEKERDLFKLILKKEDELGDRSIMF | |
| | | TVQNED (SEQ ID NO: 416) | |
| LZM009 | HC | YFGKLKSKLSIIRNLNDQVLFIDQGNRPLFEDMTDSD | CMP2005* |
| IgG4 heavy | | ARDNAPRTIFIISMYADSQPRGMAVAISVKCEKISTLS | |
| chain fused | | AENKIISFKEMNPPDNIKDTKSDIIFFQRSVPGHDNKM | |
| to IL18 | | QFESSSYEGYFLAAEKERDLFKLILKKEDELGDRSIM | |
| (IL18 in | | FTVQNEDGGGGSGGGGSGGGGSQVQLVQSGAEVKKP | |
| bold) | | GASVKVSCKASGYTFTSYYMYWVRQAPGQGLEWMGG | |
| | | VNPSNGGTNFNEKFKSRVTITADKSTSTAYMELSSLRSE | |
| | | DTAVYYCARRDYRYDMGFDYWGQGTTVTVSSASTKG | |
| | | PSVFPLAPCSRSTSESTAALGCLVKDYFPEPVTVSWNSG | |
| | | ALTSGVHTFPAVLQSSGLYSLSSVVTVPSSSLGTKTYTC | |
| | | NVDHKPSNTKVDKRVESKYGPPCPPCPAPEFLGGPSVFL | |
| | | FPPKPKDTLMISRTPEVTCVVVDVSQEDPEVQFNWYVD | |
| | | GVEVHNAKTKPREEQFNSTYRVVSVLTVLHQDWLNGK | |
| | | EYKCKVSNKGLPSSIEKTISKAKGQPREPQVYTLPPSQEE | |
| | | MTKNQVSLTCLVKGFYPSDIAVEWESNGQPENNYKTTP | |
| | | PVLDSDGSFFLYSRLTVDKSRWQEGNVFSCSVMHEALH | |
| | | NHYTQKSLSLSLGK (SEQ ID NO: 417) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |
| LZM009 | HC1 | QVQLVQSGAEVKKPGASVKVSCKASGYTFTSYYMYWV | CMP2006* |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| (T366W, | | QGTTVTVSSASTKGPSVFPLAPCSRSTSESTAALGCLVK | |
| H435R, | | DYFPEPVTVSWNSGALTSGVHTFPAVLQSSGLYSLSSVV | |
| Y436F) | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLWCLVKGFYPSDIAVEW | |
| | | ESNGQPENNYKTTPPVLDSDGSFFLYSRLTVDKSRWQE | |
| | | GNVFSCSVMHEALHNRFTQKSLSLSLGK (SEQ ID NO: | |
| | | 412) | |
| LZM009 | HC2 | YFGKLKSKLSIIRNLNDQVLFIDQGNRPLFEDMTDSD | |
| IgG4 heavy | | ARDNAPRTIFIISMYADSQPRGMAVAISVKCEKISTLS | |
| chain | | AENKIISFKEMNPPDNIKDTKSDIIFFQRSVPGHDNKM | |
| (T366S, | | QFESSSYEGYFLAAEKERDLFKLILKKEDELGDRSIM | |
| L368A, | | FTVQNEDGGGGSGGGGSGGGGSQVQLVQSGAEVKKP | |
| Y407V) | | GASVKVSCKASGYTFTSYYMYWVRQAPGQGLEWMGG | |
| fused to | | VNPSNGGTNFNEKFKSRVTITADKSTSTAYMELSSLRSE | |
| IL18 (IL18 | | DTAVYYCARRDYRYDMGFDYWGQGTTVTVSSASTKG | |
| in bold) | | PSVFPLAPCSRSTSESTAALGCLVKDYFPEPVTVSWNSG | |
| | | ALTSGVHTFPAVLQSSGLYSLSSVVTVPSSSLGTKTYTC | |
| | | NVDHKPSNTKVDKRVESKYGPPCPPCPAPEFLGGPSVFL | |
| | | FPPKPKDTLMISRTPEVTCVVVDVSQEDPEVQFNWYVD | |
| | | GVEVHNAKTKPREEQFNSTYRVVSVLTVLHQDWLNGK | |
| | | EYKCKVSNKGLPSSIEKTISKAKGQPREPQVYTLPPSQEE | |
| | | MTKNQVSLSCAVKGFYPSDIAVEWESNGQPENNYKTTP | |
| | | PVLDSDGSFFLVSRLTVDKSRWQEGNVFSCSVMHEALH | |
| | | NHYTQKSLSLSLGK (SEQ ID NO: 418) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |
| LZM009 | HC1 | QVQLVQSGAEVKKPGASVKVSCKASGYTFTSYYMYWV | CMP2007* |
| IgG4 heavy | | RQAPGQGLEWMGGVNPSNGGTNFNEKFKSRVTITADK | |
| chain | | STSTAYMELSSLRSEDTAVYYCARRDYRYDMGFDYWG | |
| (T366W, | | QGTTVTVSSASTKGPSVFPLAPCSRSTSESTAALGCLVK | |
| H435R, | | DYFPEPVTVSWNSGALTSGVHTFPAVLQSSGLYSLSSVV | |
| Y436F) | | TVPSSSLGTKTYTCNVDHKPSNTKVDKRVESKYGPPCPP | |
| | | CPAPEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQP | |
| | | REPQVYTLPPSQEEMTKNQVSLWCLVKGFYPSDIAVEW | |
| | | ESNGQPENNYKTTPPVLDSDGSFFLYSRLTVDKSRWQE | |
| | | GNVFSCSVMHEALHNRFTQKSLSLSLGK (SEQ ID NO: | |
| | | 412) | |
| IL18-Fc | HC2 | YFGKLKSKLSIIRNLNDQVLFIDQGNRPLFEDMTDSD | |
| fusion | | ARDNAPRTIFIISMYADSQPRGMAVAISVKCEKISTLS | |
| (T366S, | | AENKIISFKEMNPPDNIKDTKSDIIFFQRSVPGHDNKM | |
| L368A, | | QFESSSYEGYFLAAEKERDLFKLILKKEDELGDRSIM | |
| Y407V) | | FTVQNEDGGGGSGGGGSGGGGSESKYGPPCPPCPAPEF | |
| (IL18 in | | LGGPSVFLFPPKPKDTLMISRTPEVTCVVVDVSQEDPEV | |
| bold) | | QFNWYVDGVEVHNAKTKPREEQFNSTYRVVSVLTVLH | |
| | | QDWLNGKEYKCKVSNKGLPSSIEKTISKAKGQPREPQV | |
| | | YTLPPSQEEMTKNQVSLSCAVKGFYPSDIAVEWESNGQ | |
| | | PENNYKTTPPVLDSDGSFFLVSRLTVDKSRWQEGNVFS | |
| | | CSVMHEALHNHYTQKSLSLSLGK (SEQ ID NO: 419) | |
| LZM009 | LC | EIVLTQSPATLSLSPGERATISCRASKGVSTSGYSYLHWY | |
| light chain | | QQKPGQAPRLLIYLASYLESGVPARFSGSGSGTDFTLTIS | |
| | | SLEPEDFATYYCQHSRELPLTFGTGTKVEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKVY | |
| | | ACEVTHQGLSSPVTKSFNRGEC (SEQ ID NO: 411) | |

*CMP numbers refer to the IL-18 containing fusion proteins.

Although the present disclosure and its advantages have been described in detail, it should be understood that various changes, substitutions and alterations can be made herein without departing from the spirit and scope of the disclosure as defined in the appended claims.

1. A fusion immunocytokine, comprising:
an IL-18 polypeptide fused to an antibody or an antigen binding fragment thereof specific for an immune cell associated antigen, optionally by a peptide linker.

2. The fusion immunocytokine of claim 1, wherein the IL-18 polypeptide is fused via its N-terminus to the antibody or antigen binding fragment thereof.

3. The fusion immunocytokine of claim 2, wherein the IL-18 polypeptide is fused to a C-terminus of a light chain or a heavy chain, or a fragment thereof, of the antibody or antigen binding fragment thereof.

4. The fusion immunocytokine of claim 3, wherein the light chain or heavy chain is a full-length light chain or full length-heavy chain.

5. The fusion immunocytokine of claim 1, wherein the IL-18 polypeptide is fused via its C-terminus to the antibody or antigen binding fragment thereof.

6. The fusion immunocytokine of claim 5, wherein the IL-18 polypeptide is fused to an N-terminus of a light chain or a heavy chain, or a fragment thereof, of the antibody or antigen binding fragment thereof.

7. The fusion immunocytokine of claim 6, wherein the light chain or heavy chain is a full-length light chain or full-length heavy chain.

8. The fusion immunocytokine of claim 1, comprising:
a) a first polypeptide comprising an antigen binding domain of the antibody or antigen binding fragment thereof; and
b) a second polypeptide comprising the IL-18 polypeptide fused to a fragment crystallizable (Fc) domain.

9. The fusion immunocytokine of claim 8, wherein the first polypeptide comprises a full-length heavy chain of the antibody.

10. The fusion immunocytokine of claim 8, wherein the first polypeptide is bound to a light chain of the antibody or antigen binding fragment thereof.

11. The fusion immunocytokine of claim 8, wherein the IL-18 polypeptide is fused to a C-terminus of the Fc domain.

12. The fusion immunocytokine of claim 8, wherein the second polypeptide does not contain an antigen-binding domain.

13-31. (canceled)

32. The fusion immunocytokine of claim 1, wherein the immune cell associated antigen is 4-IBB, CD3, CCR8, CD8A, CD8B, CD16A, CD28, CD80, CD86, CD96, CD226, CTLA-4, D40, GITR, ICOS, LAG-3,MHCI, MHCII, NKG2A, NKG2D, NKp30, NKp44, NKp46, OX40, PD-1, PD-L1, PD-L2, SIRPA, TCR, TIGIT, TIM-3, or VISTA.

33. The fusion immunocytokine of claim 1, wherein the immune cell associated antigen is PD-1.

34-36. (canceled)

37. The fusion immunocytokine of claim 33, wherein the antibody or antigen binding fragment thereof comprises a VH and VL of SEQ ID NOs: 376 and 377.

38-42. (canceled)

43. The fusion immunocytokine of claim 421, wherein the antibody or antigen binding fragment thereof comprises a VH and VL set forth in Table 1, or a VH and VL each having at least about 80%, 85%, 90%, 95%, or 98% sequence identity to the VH and VL set forth in Table 1.

44. The fusion immunocytokine of claim 1, wherein the IL-18 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 1.

45. (canceled)

46. The fusion immunocytokine of claim 1, wherein the IL-18 polypeptide comprises the amino acid sequence of SEQ ID NO: 30.

47-59. (canceled)

60. A method of treating cancer in a subject in need thereof,
comprising administering to the subject an effective amount of a fusion immunocytokine comprising an IL-18 polypeptide fused to an anti-PD-1 antibody or an antigen binding fragment thereof, optionally by a peptide linker.

61-66. (canceled)

67. A method of manufacturing a fusion immunocytokine, wherein the fusion immunocytokine comprises an IL-18 polypeptide fused to an antibody or an antigen binding fragment thereof specific for an immune cell associated antigen, optionally by a peptide linker, the method comprising:
expressing the fusion immunocytokine in a host cell.
